SEQUENCE LISTING (58 sequences)

SEQ ID NO: 1 - DNA, 20 bp, Artificial Sequence
  primer PLK1 RT F
cacagtgtcaatgcctccaa

SEQ ID NO: 2 - DNA, 19 bp, Artificial Sequence
  primer PLK1 RT R
gacccagaagatggggatg

SEQ ID NO: 3 - DNA, 20 bp, Artificial Sequence
  primer ACTB RT F
ctcttccagccttccttcct

SEQ ID NO: 4 - DNA, 20 bp, Artificial Sequence
  primer ACTB RT R
ggatgtccacgtcacacttc

SEQ ID NO: 5 - DNA, 18 bp, Artificial Sequence
  primer PLK1 TSS ChIP F
gcgcaggcttttgtaacg

SEQ ID NO: 6 - DNA, 19 bp, Artificial Sequence
  primer PLK1 TSS ChIP R
ctcctccccgaattcaaac

SEQ ID NO: 7 - DNA, 21 bp, Artificial Sequence
  primer PLK1 shRNA1
gttctttacttctggctatat

SEQ ID NO: 8 - DNA, 21 bp, Artificial Sequence
  primer PLK1 shRNA2
ctgcaccgaaaccgagttatt

SEQ ID NO: 9 - DNA, 18 bp, Artificial Sequence
  primer E2F2 F
actggaagtgcccgacag

SEQ ID NO: 10 - DNA, 20 bp, Artificial Sequence
  primer E2F2 R
tcctctgggcacaggtagac

SEQ ID NO: 11 - DNA, 20 bp, Artificial Sequence
  primer CKS1B F
atcttggcgttcagcagagt

SEQ ID NO: 12 - DNA, 20 bp, Artificial Sequence
  primer CKS1B R
cggaacagcaagatgtgagg

SEQ ID NO: 13 - DNA, 20 bp, Artificial Sequence
  primer RUSC1 F
agcagttggagctgtggttt

SEQ ID NO: 14 - DNA, 20 bp, Artificial Sequence
  primer RUSC1 R
atcctgttggcaggtacagg

SEQ ID NO: 15 - DNA, 20 bp, Artificial Sequence
  primer MSN F
aagcagatggagcgtgctat

SEQ ID NO: 16 - DNA, 20 bp, Artificial Sequence
  primer MSN R
cctacgggtctgttcttcca

SEQ ID NO: 17 - DNA, 19 bp, Artificial Sequence
  primer NUP93 F
agtgcgcatggagtttgtc

SEQ ID NO: 18 - DNA, 22 bp, Artificial Sequence
  primer NUP93 R
tgcaaatttccaaagacagtca

SEQ ID NO: 19 - DNA, 21 bp, Artificial Sequence
  primer PRKDC F
tcgcaccttactctgttgaaa

SEQ ID NO: 20 - DNA, 20 bp, Artificial Sequence
  primer PRKDC R
ccagggctggaattttacat

SEQ ID NO: 21 - DNA, 20 bp, Artificial Sequence
  primer KIF4A F
ggtcagacagcccagatgtt

SEQ ID NO: 22 - DNA, 20 bp, Artificial Sequence
  primer KIF4A R
gctcttctagcttggcgttc

SEQ ID NO: 23 - DNA, 20 bp, Artificial Sequence
  primer TPX2 F
cgaaagcatccttcatctcc

SEQ ID NO: 24 - DNA, 20 bp, Artificial Sequence
  primer TPX2 R
tccttgggacaggttgaaag

SEQ ID NO: 25 - DNA, 20 bp, Artificial Sequence
  primer KIF4A F
ggtcagacagcccagatgtt

SEQ ID NO: 26 - DNA, 20 bp, Artificial Sequence
  primer KIF4A R
gctcttctagcttggcgttc

SEQ ID NO: 27 - DNA, 22 bp, Artificial Sequence
  primer MCM4 F
atgagaaacctgaatccagaag

SEQ ID NO: 28 - DNA, 20 bp, Artificial Sequence
  primer MCM4 R
atcagctgggatgtcctgat

SEQ ID NO: 29 - DNA, 21 bp, Artificial Sequence
  primer MCM7 F
gcgtcactggtattttcttgc

SEQ ID NO: 30 - DNA, 20 bp, Artificial Sequence
  primer MCM7 R
atgggcttccaggtaggttt

SEQ ID NO: 31 - DNA, 20 bp, Artificial Sequence
  primer BUB1 F
acggagaaagcatgagcaat

SEQ ID NO: 32 - DNA, 22 bp, Artificial Sequence
  primer BUB1 R
caaaagcatttgcttctttcct

SEQ ID NO: 33 - DNA, 20 bp, Artificial Sequence
  primer TOP2A F
gctggatccaccaaagatgt

SEQ ID NO: 34 - DNA, 24 bp, Artificial Sequence
  primer TOP2A R
catgtccacataactacgaaatcc

SEQ ID NO: 35 - DNA, 21 bp, Artificial Sequence
  primer NUF2 F
caagcagctttcagatggaat

SEQ ID NO: 36 - DNA, 20 bp, Artificial Sequence
  primer NUF2 R
gaatttccctcttgcagcac

SEQ ID NO: 37 - DNA, 18 bp, Artificial Sequence
  primer MCM2 F
agagaggcctggctctgg

SEQ ID NO: 38 - DNA, 20 bp, Artificial Sequence
  primer MCM2 R
caccacgtaccttgtgcttg

SEQ ID NO: 39 - DNA, 25 bp, Artificial Sequence
  primer HELLS F
cagaagaagaaagaaaaattggaga

SEQ ID NO: 40 - DNA, 20 bp, Artificial Sequence
  primer HELLS R
tggcttctcttcacttgcat

SEQ ID NO: 41 - DNA, 20 bp, Artificial Sequence
  primer CENPE F
ggagttatacccagggcaat

SEQ ID NO: 42 - DNA, 23 bp, Artificial Sequence
  primer CENPE R
cacgtaagagaaattccctatca

SEQ ID NO: 43 - DNA, 20 bp, Artificial Sequence
  primer MCM3 F
gatcagcaggacacccagat

SEQ ID NO: 44 - DNA, 20 bp, Artificial Sequence
  primer MCM3 R
tgctgcactcaccatcttct

SEQ ID NO: 45 - DNA, 21 bp, Artificial Sequence
  primer RBL1 F
gaaccaccaaagttaccacga

SEQ ID NO: 46 - DNA, 25 bp, Artificial Sequence
  primer RBL1 R
tccaacagaaattaaacagatcctt

SEQ ID NO: 47 - DNA, 20 bp, Artificial Sequence
  primer NONO F
gcaaaaacgaaagcaactgg

SEQ ID NO: 48 - DNA, 20 bp, Artificial Sequence
  primer NONO R
cgcatcatttcttcttgctg

SEQ ID NO: 49 - DNA, 20 bp, Artificial Sequence
  primer POLA1 F
atcgagcgaacgctttactt

SEQ ID NO: 50 - DNA, 20 bp, Artificial Sequence
  primer POLA1 R
ttcctgtttctttccccgta

SEQ ID NO: 51 - DNA, 20 bp, Artificial Sequence
  primer TAGLN2 F
gacgcgagaacttccagaac

SEQ ID NO: 52 - DNA, 20 bp, Artificial Sequence
  primer TAGLN2 R
cctcggggtacagtgcatta

SEQ ID NO: 53 - DNA, 21 bp, Artificial Sequence
  primer CKS1B shRNA1
gacccacagcctaagctgagt

SEQ ID NO: 54 - DNA, 21 bp, Artificial Sequence
  primer E2F2 shRNA2
gtacgggtgaggagtggataa

SEQ ID NO: 55 - DNA, 21 bp, Artificial Sequence
  primer BRD4 shRNA1
gacgtgggaggaaagaaacag

SEQ ID NO: 56 - DNA, 21 bp, Artificial Sequence
  primer BRD4 shRNA2
gtgctgacgtccgattgatgt

SEQ ID NO: 57 - DNA, 21 bp, Artificial Sequence
  primer BRD4 shRNA3
cgcaagctccaggatgtgttc

SEQ ID NO: 58 - DNA, 21 bp, Artificial Sequence
  primer BRD4 shRNA4
gctcctctgacagcgaagact

(19) United States
(12) Patent Application Publication
(10) Pub. No.: US 2023/0255975 A1
(43) Pub. Date: Aug. 17, 2023

(54) METHODS FOR TREATING CANCER WITH COMBINATION THERAPIES

(71) Applicant: Celgene Corporation, Summit, NJ (US)

(72) Inventors: Aarif AHSAN, Hillsborough, NJ (US); Kamlesh BISHT, Hillsborough Township, NJ (US); Chad BJORKLUND, Summit, NJ (US); Jennifer Erin FLYNT, Hattiesburg, MS (US); Chih-Chao HSU, Summit, NJ (US); Danny Vijey JEYARAJU, Florham Park, NJ (US); Maria ORTIZ-ESTEVEZ, Donostia (ES); William Edward PIERCEALL, Madison, NJ (US); Anjan THAKURTA, Basking Ridge, NJ (US); Fadi George TOWFIC, La Jolla, CA (US)

(21) Appl. No.: 18/012,414

(22) PCT Filed: Jun. 24, 2021
(86) PCT No.: PCT/US2021/038870

§ 371 Date: Dec. 22, 2022

Related U.S. Application Data
(60) Provisional application No. 63/044,127, filed on Jun. 25, 2020.

(51) Int. Cl. A61K 31/5377; A61K 31/519; A61K 31/551; A61K 31/416; A61K 31/381; A61K 31/44; A61P 35/00

(52) U.S. Cl. CPC A61K 31/5377; A61K 31/519; A61K 31/551; A61K 31/416; A61K 31/381; A61K 31/44; A61P 35/00

(57) ABSTRACT

Provided herein are methods of using a compound provided herein (e.g., Compound 1, Compound 2, Compound 3, Compound 4, Compound 5, Compound 6, or Compound 7, or a stereoisomer or mixture of stereoisomers, pharmaceutically acceptable salt, tautomer, prodrug, solvate, hydrate, co-crystal, clathrate, or polymorph thereof), in combination with a second) active agent for treating cancer. The second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRCS inhibitor, or a DNA methyltransferase inhibitor.

### 1. CROSS-REFERENCE TO RELATED APPLICATIONS

This application claims priority to U.S. Provisional Application No. 63/044,127, filed on Jun. 25, 2020, the entirety of which is incorporated herein by reference.

### 2. SEQUENCE LISTING

The present specification is being filed with a computer readable form (CRF) copy of the Sequence Listing. The CRF is entitled 14247-544-228_Seqlisting_ST25.txt, which was created on Jun. 21, 2021 and is 11,150 bytes in size, and is incorporated herein by reference in its entirety.

### 3. FIELD

Provided herein are methods of using a compound provided herein (e.g., Compound 1, Compound 2, Compound 3, Compound 4, Compound 5, Compound 6, or Compound 7, or a stereoisomer or mixture of stereoisomers, pharmaceutically acceptable salt, tautomer, prodrug, solvate, hydrate, co-crystal, clathrate, or polymorph thereof), in combination with a second active agent for treating cancer.

### 4. BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and metastasis. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Current cancer therapy may involve surgery, chemotherapy, hormonal therapy and/or radiation treatment to eradicate neoplastic cells in a patient. Recent advances in cancer therapeutics are discussed by Rajkumar et al. in Nature Reviews Clinical Oncology 11, 628-630 (2014).

All of the current cancer therapy approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells.

Despite availability of a variety of chemotherapeutic agents, chemotherapy has many drawbacks. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove or become refractory to standard chemotherapeutic treatment protocols.

Hematological malignancies are cancers that begin in blood-forming tissue, such as the bone marrow, or in the cells of the immune system. Examples of hematological malignancies are leukemia, lymphoma, and myeloma. More specific examples of hematological malignancies include but are not limited to acute myeloid leukemia (AML), acute lymphocytic leukemia (ALL), multiple myeloma (MM), non-Hodgkin's lymphoma (NHL), diffuse large B-cell lymphoma (DLBCL), Hodgkin's lymphoma (HL), T-cell lymphoma (TCL), Burkitt lymphoma (BL), chronic lymphocytic leukemia/small lymphocytic lymphoma (CLL/SLL), marginal zone lymphoma (MZL), and myelodysplastic syndromes (MDS).

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin, except in some patients (estimated at 1% to 5%) whose myeloma cells do not secrete these proteins (termed non-secretory myeloma). M-protein, short for monoclonal protein, also known as paraprotein, is a particularly abnormal protein produced by the myeloma plasma cells and can be found in the blood or urine of almost all patients with multiple myeloma, except for patients who have non-secretory myeloma or whose myeloma cells produce immunoglobulin light chains with heavy chain.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

### 5. SUMMARY

Provided herein are methods of using a compound provided herein (e.g., Compound 1, Compound 2, Compound 3, Compound 4, Compound 5, Compound 6, or Compound 7, or a stereoisomer or mixture of stereoisomers, pharmaceutically acceptable salt, tautomer, prodrug, solvate, hydrate, co-crystal, clathrate, or polymorph thereof), in combination with a second active agent for treating cancer, wherein the second active agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

Also provided for use in the methods provided herein are pharmaceutical compositions formulated for administration by an appropriate route and means containing effective concentrations of the compounds provided herein, for example, Compound 1, Compound 2, Compound 3, Compound 4, Compound 5, Compound 6, or Compound 7, or a stereoisomer or mixture of stereoisomers, pharmaceutically acceptable salt, tautomer, prodrug, solvate, hydrate, co-crystal, clathrate, or polymorph thereof, and optionally comprising at least one pharmaceutical carrier. In one embodiment, the pharmaceutical compositions deliver amounts of the compound effective for the treatment of a cancer provided herein in combination with the second active agent provided herein.

In one embodiment, the cancer is a hematological malignancy. In one embodiment, the cancer is multiple myeloma (MM).

The compounds or compositions provided herein, or pharmaceutically acceptable derivatives thereof, may be administered simultaneously with, prior to, or after administration of each other and one or more of the above therapies.

These and other aspects of the subject matter described herein will become evident upon reference to the following detailed description.

### 6. BRIEF DESCRIPTION OF THE FIGURES

FIGS. 1A to 1D show PLK1 association with PFS in MMRF, with OS in MMRF, with PFS in MM010, and with OS in MM010, respectively.

FIG. 1E shows that PLK1 expression was significantly upregulated in patients at relapse.

FIG. 1F shows the expression pattern of PLK1 across various stages of MM disease progression and relapse.

FIG. 2A and FIG. 2B show the effects of pomalidomide treatment in PLK1 levels and its downstream effector pCDC25C and CDC25C in EJM and EJM/PR cell lines, respectively.

FIG. 2C shows the effects of pomalidomide and Compound 5 treatments in PLK1 levels and its downstream effector pCDC25C and CDC25C in MM1.S cell line.

FIG. 2D shows the effects of pomalidomide treatment in PLK1 transcript levels in MM1.S cells; FIG. 2E shows the effects of pomalidomide treatment in the binding of Aiolos and Ikaros to transcriptional start sites (TSS) of PLKL.

FIG. 2F shows that both Aiolos and Ikaros knock down lead to a decrease in PLK1 levels.

FIG. 3 shows the changes in PLK1 signaling after treating cells with Nocodazole and Compound 5 and their combination.

FIG. 4A shows the levels of PLK1, CDC25C and pCDC25C and cereblon in six pomalidomide sensitive and resistant isogenic pair of cell lines.

FIG. 4B shows an increased proportion of G2-M cells in five of six pomalidomide resistant cell lines.

FIG. 5A shows treatment of AMO1 cell lines with Compound 5 in combination with BI2536; FIG. 5B shows the corresponding combination index values; FIG. 5C shows the effect of treatment of AMO1-PR cell lines with Compound 5 in combination with BI2536; and FIG. 5D shows the corresponding combination index values.

FIG. 5E shows treatment of K12PE cell lines with Compound 5 in combination with BI2536; FIG. 5F shows the corresponding combination index values; FIG. 5G shows the effect of treatment of K12PE/PR cell lines with Compound 5 in combination with BI2536; and FIG. 5H shows the corresponding combination index values.

FIG. 5I and FIG. 5J show the effects of Compound 5 in combination with BI2536 in early and late apoptosis in AMO1 and AMO1-PR cells, respectively.

FIG. 5K shows changes in Ikaros and pro-survival signaling in AMO1 and AMO1-PR cell lines in response to BI2536 and Compound 5 after treatment.

FIG. 6A shows treatment of Mc-CAR cell with Compound 5 in combination with BI2536; FIG. 6B shows the corresponding combination index values.

FIG. 6C shows changes in Aiolos and Ikaros levels in Mc-CAR cell line in response to BI2536 and Compound 5 after treatment.

FIG. 7A shows that patients who harbored biallelic P53 demonstrated significantly elevated expression of PLK1.

FIG. 7B shows the effects of BI2536 in biallelic P53 cell line K12PE and P53-wild type AMO1 cells.

FIG. 8A and FIG. 8B show that E2F2, CKS1B, TOP2A and NUF2 were up-regulated in MDMS8-like cell line at the protein and transcript expression levels, respectively.

FIGS. 9A to 9D show CKS1B association with OS, CKS1B association with PFS, E2F2 association with OS, and E2F2 association with PFS, respectively.

FIG. 9E shows that knock-down of CKS11B and E2F2 demonstrated a significant decrease in proliferation and increase in apoptosis.

FIG. 10A and FIG. 10B show the effects of BRD4 inhibitors on CKS1B and E2F2 and their target genes in DF15PR and H929 cell lines, respectively.

FIGS. 10C to 10F show the effects of BRD4 inhibitors on transcript level of CKS1B in DF15PR cell line, on transcript level of E2F2 in DF15PR cell line, on transcript level of CKS1B in H929 cell line, and on transcript level of E2F2 in H929 cell line, respectively.

FIG. 11A and FIG. 11B show that four different shRNA targeting BRD4 consistently demonstrated a decrease in CKS1B and E2F2 levels in K12PE and DF15PR cell lines, respectively; FIG. 11C and FIG. 11D show that all the four shRNAs caused a marked decrease in cell proliferation in in K12PE and MDMS8-like cells, respectively.

FIG. 12 shows effects of pomalidomide on CKS1B and E2F2 in Pom sensitive and resistant cell lines.

FIG. 13A shows treatment of K12PE cell lines with Len in combination with JQ1; FIG. 13B shows the corresponding combination index values; FIG. 13C shows treatment of K12PE cell lines with Pom in combination with JQ1; FIG. 13D shows the corresponding combination index values; FIG. 13E shows treatment of K12PE cell lines with Compound 5 in combination with JQ1; FIG. 13F shows the corresponding combination index values; FIG. 13G shows treatment of K12PE cell lines with Compound 6 in combination with JQ1; FIG. 13H shows the corresponding combination index values.

FIG. 13I shows treatment of K12PE/PR cell lines with Len in combination with JQ1; FIG. 13J shows the corresponding combination index values; FIG. 13K shows treatment of K12PE/PR cell lines with Pom in combination with JQ1; FIG. 13L shows the corresponding combination index values; FIG. 13M shows treatment of K12PE/PR cell lines with Compound 5 in combination with JQ1; FIG. 13N shows the corresponding combination index values; FIG. 13O shows treatment of K12PE/PR cell lines with Compound 6 in combination with JQ1; FIG. 13P shows the corresponding combination index values.

FIG. 13Q shows the effects on the levels of Aiolos, Ikaros, CKS1B, E2F2, Myc, Survivin by treatment of combination of JQ1 and Len, Pom, Compound 5, and Compound 6.

FIG. 14A and FIG. 14B show the association of NEK2 expression with progression free and overall survival, respectively.

FIG. 14C shows that NEK2 expression was significantly upregulated in patients at relapse.

FIG. 14D shows significant upregulation of NEK2 expression in pomalidomide resistant cell lines.

FIGS. 15A to 15F show NEK2 association with PFS in MMRF, with OS in MMRF, with PFS in DFCI, with OS in DFCI, with PFS in MM0010, and with OS in MM0010, respectively.

FIG. 16A shows treatment of AMO1 cell lines with Compound 5 in combination with rac-CCT 250863; FIG. 16B shows the corresponding combination index values; FIG. 16C shows treatment of AMO1/PR cell lines with Compound 5 in combination with rac-CCT 250863; FIG. 16D shows the corresponding combination index values; FIG. 16E shows treatment of AMO1 cell lines with Compound 6 in combination with rac-CCT 250863; FIG. 16F shows the corresponding combination index values; FIG. 16G shows treatment of AMO1/PR cell lines with Compound 6 in combination with rac-CCT 250863; FIG. 16H shows the corresponding combination index values; FIG. 16I shows treatment of AMO1 cell lines with Compound 5 in combination with JH295; FIG. 16J shows the corresponding combination index values; FIG. 16K shows treatment of AMO1/PR cell lines with Compound 5 in combination with JH295; FIG. 16L shows the corresponding combination index values; FIG. 16M shows treatment of AMO1 cell lines with Compound 6 in combination with JH295; FIG. 16N shows the corresponding combination index values; FIG. 16O shows treatment of AMO1/PR cell lines with Compound 6 in combination with JH295; FIG. 16P shows the corresponding combination index values.

FIG. 17 shows increase in apoptotic cells when NEK2 knock down was combined with Compound 5 or Compound 6.

FIG. 18A and FIG. 18B show the effects of trametinib in combination with Len in AMO1 and AMO1-PR cell lines, respectively; FIG. 18C and FIG. 18D show the effects of trametinib in combination with Pom in AMO1 and AMO1-PR cell lines, respectively;

FIG. 18E and FIG. 18F show the effects of trametinib in combination with Compound 5 in AMO1 and AMO1-PR cell lines, respectively; FIG. 18G and FIG. 18H show the effects of trametinib in combination with Compound 6 in AMO1 and AMO1-PR cell lines, respectively.

FIG. 19 shows that trametinib and Compound 6 combination synergistically decreased ERK, ETV4 and MYC signaling in AMO1-PR cell line.

FIG. 20A and FIG. 20B show the effects of trametinib and Compound 6 combination on apoptosis in AMO1 and AMO1-PR cell lines at Day 3 and Day 5, respectively.

FIG. 21A and FIG. 21B show the effects of trametinib and Compound 6 combination on cell cycles in AMO1-PR cell line at Day 3 and Day 5, respectively.

FIG. 22A and FIG. 22B show that patients with high expression of BIRC5 demonstrated poorer PFS and OS, respectively.

FIG. 23A shows that several pomalidomide resistant cell lines demonstrated increase expression of BIRC5; FIG. 23B shows that BIRC5 levels decreased in response to Compound 5 treatment at 48 and 72 hours, followed by an onset of apoptosis in MM1.S cell line.

FIG. 24A shows treatment of AMO1 cell lines with Compound 5 in combination with YM155; FIG. 24B shows the corresponding combination index values;

FIG. 24C shows treatment of AMO1/PR cell lines with Compound 5 in combination with YM155; FIG. 24D shows the corresponding combination index values; FIG. 24E shows treatment of AMO1 cell lines with Compound 6 in combination with YM155; FIG. 24F shows the corresponding combination index values; FIG. 24G shows treatment of AMO1/PR cell lines with Compound 6 in combination with YM155; FIG. 24H shows the corresponding combination index values.

FIG. 25A shows that BIRC5 knock-down decreased the proliferation of AMO1-PR cells; FIG. 25B shows that BIRC5 knock-down also downregulated the expression of high risk associated gene, FOXM1.

FIG. 26A shows that high risk associated genes, BIRC5 and FOXM1 demonstrated significant co-expression in myeloma genome project, suggesting their co-regulation; FIG. 26B shows that inhibition of BIRC5 by YM155 also downregulated FOXM1 expression in a dose dependent manner in AMO1-PR and K12PE-PR cell lines.

### 7. DETAILED DESCRIPTION OF THE INVENTION

### A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the term “pharmaceutically acceptable salts” refers to salts prepared from pharmaceutically acceptable, relatively non-toxic acids, including inorganic acids and organic acids. In certain embodiments, suitable acids include, but are not limited to, acetic, adipic, 4-aminosalicylic, ascorbic, aspartic, benzenesulfonic, benzoic, camphoric, camphorsulfonic, capric, caproic, caprylic, cinnamic, carbonic, citric, cyclamic, dihydrogenphosphoric, 2,5-dihydroxybenzoic (gentisic), 1,2-ethanedisulfonic, ethanesulfonic, fumaric, galactunoric, gluconic, glucuronic, glutamic, glutaric, glycolic, hippuric, hydrobromic, hydrochloric, hydriodic, isobutyric, isethionic, lactic, maleic, malic, malonic, mandelic, methanesulfonic, monohydrogencarbonic, monohydrogen-phosphoric, monohydrogensulfuric, mucic, 1,5-naphthalenedisulfonic, nicotinic, nitric, oxalic, pamoic, pantothenic, phosphoric, phthalic, propionic, pyroglutamic, salicylic, suberic, succinic, sulfuric, tartaric, toluenesulfonic acid, and the like (see, e.g., S. M. Berge et al., J. Pharm. Sci., 66:1-19 (1977); and Handbook of Pharmaceutical Salts: Properties, Selection and Use, P. H. Stahl and C. G. Wermuth, Eds., (2002), Wiley, Weinheim). In certain embodiments, suitable acids are strong acids (e.g., with pKa less than about 1), including, but not limited to, hydrochloric, hydrobromic, sulfuric, nitric, methanesulfonic, benzene sulfonic, toluene sulfonic, naphthalene sulfonic, naphthalene disulfonic, pyridine-sulfonic, or other substituted sulfonic acids. Also included are salts of other relatively non-toxic compounds that possess acidic character, including amino acids, such as aspartic acid and the like, and other compounds, such as aspirin, ibuprofen, saccharin, and the like. Acid addition salts can be obtained by contacting the neutral form of a compound with a sufficient amount of the desired acid, either neat or in a suitable solvent.

As used herein, and unless otherwise specified, the term “prodrug” of an active compound refers to compounds that are transformed in vivo to yield the active compound or a pharmaceutically acceptable form of the active compound. A prodrug can be inactive when administered to a subject, but is converted in vivo to an active compound, for example, by hydrolysis (e.g., hydrolysis in blood). Prodrugs include compounds wherein a hydroxy, amino or mercapto group is bonded to any group that, when the prodrug of the active compound is administered to a subject, cleaves to form a free hydroxy, free amino or free mercapto group, respectively.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other. The absolute stereochemistry can be specified according to the Cahn-Ingold-Prelog R-S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. However, the sign of optical rotation, (+) and (−), is not related to the absolute configuration of the molecule, R and S. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

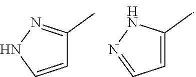

It should also be noted a compound described herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of a compound described herein, whether radioactive or not, are intended to be encompassed within the scope of the embodiments provided herein. In some embodiments, there are provided isotopologs of a compound described herein, for example, the isotopologs are deuterium, carbon-13, and/or nitrogen-15 enriched. As used herein, “deuterated”, means a compound wherein at least one hydrogen (H) has been replaced by deuterium (indicated by D or ²H), that is, the compound is enriched in deuterium in at least one position.

It should be noted that if there is a discrepancy between a depicted structure and a name for that structure, the depicted structure is to be accorded more weight.

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” in connection with a compound means an amount capable of treating, preventing, or managing a disorder, disease or condition, or symptoms thereof.

As used herein and unless otherwise indicated, the term “subject” or “patient” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

As used herein and unless otherwise indicated, the term “relapsed” refers to a disorder, disease, or condition that responded to treatment (e.g., achieved a complete response) then had progression. The treatment can include one or more lines of therapy. In one embodiment, the disorder, disease or condition has been previously treated with one or more lines of therapy. In another embodiment, the disorder, disease or condition has been previously treated with one, two, three or four lines of therapy. In some embodiments, the disorder, disease or condition is a hematological malignancy.

As used herein and unless otherwise indicated, the term “refractory” refers to a disorder, disease, or condition that has not responded to prior treatment that can include one or more lines of therapy. In one embodiment, the disorder, disease, or condition has been previously treated one, two, three or four lines of therapy. In one embodiment, the disorder, disease, or condition has been previously treated with two or more lines of treatment, and has less than a complete response (CR) to most recent systemic therapy containing regimen. In some embodiments, the disorder, disease or condition is a hematological malignancy.

In the context of a cancer, for example, a hematological malignancy, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not include deaths. In one embodiment, PFS means the time from treatment onset until tumor progression or death. In one embodiment, PFS means the time from the first dose of compound to the first occurrence of disease progression or death from any cause. In one embodiment, PFS rates are computed using the Kaplan-Meier estimates. Event-free survival (EFS) means the time from treatment onset until any treatment failure, including disease progression, treatment discontinuation for any reason, or death. In one embodiment, overall response rate (ORR) means the percentage of patients who achieve a response. In one embodiment, ORR means the sum of the percentage of patients who achieve complete and partial responses. In one embodiment, ORR means the percentage of patients whose best response≥partial response (PR). In one embodiment, duration of response (DoR) is the time from achieving a response until relapse or disease progression. In one embodiment, DoR is the time from achieving a response≥partial response (PR) until relapse or disease progression. In one embodiment, DoR is the time from the first documentation of a response until to the first documentation of progressive disease or death. In one embodiment, DoR is the time from the first documentation of a response≥partial response (PR) until to the first documentation of progressive disease or death. In one embodiment, time to response (TTR) means the time from the first dose of compound to the first documentation of a response. In one embodiment, TTR means the time from the first dose of compound to the first documentation of a response≥partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

As used herein “multiple myeloma” refers to hematological conditions characterized by malignant plasma cells and includes the following disorders: monoclonal gammopathy of undetermined significance (MGUS); low risk, intermediate risk, and high risk multiple myeloma; newly diagnosed multiple myeloma (including low risk, intermediate risk, and high risk newly diagnosed multiple myeloma); transplant eligible and transplant ineligible multiple myeloma; smoldering (indolent) multiple myeloma (including low risk, intermediate risk, and high risk smouldering multiple myeloma); active multiple myeloma; solitary plasmacytoma; extramedullary plasmacytoma; plasma cell leukemia; central nervous system multiple myeloma; light chain myeloma; non-secretory myeloma; Immunoglobulin D myeloma; and Immunoglobulin E myeloma; and multiple myeloma characterized by genetic abnormalities, such as Cyclin D translocations (for example, t(11;14)(q13;q32); t(6;14)(p21;32); t(12;14)(p13;q32); or t(6;20);); MMSET translocations (for example, t(4;14)(p16;q32)); MAF translocations (for example, t(14;16)(q32;q32); t(20;22); t(16; 22)(q11;q13); or t(14;20)(q32;q11)); or other chromosome factors (for example, deletion of 17p13, or chromosome 13; del(17/17p), nonhyperdiploidy, and gain(1q)). In one embodiment, the multiple myeloma is characterized according to the multiple myeloma International Staging System (ISS). In one embodiment, the multiple myeloma is Stage I multiple myeloma as characterized by ISS (e.g., serum β2 microglobulin<3.5 mg/L and serum albumin≥3.5 g/dL). In one embodiment, the multiple myeloma is Stage III multiple myeloma as characterized by ISS (e.g., serum β2 microglobulin>5.4 mg/L). In one embodiment, the multiple myeloma is Stage II multiple myeloma as characterized by ISS (e.g., not Stage I or III).

In certain embodiments, the treatment of multiple myeloma may be assessed by the International Uniform Response Criteria for Multiple Myeloma (IURC) (see Dune B G M, Harousseau J-L, Miguel J S, etap. International uniform response criteria for multiple myeloma. Leukemia, 2006; (10) 10: 1-7), using the response and endpoint definitions shown below:

| Response | |
|---|---|
| Subcategory | Response Criteriaa |
| sCR | CR as defined below plus Normal FLC ratio and |
| | Absence of clonal cells in bone marrow^{b} by |
| | immunohistochemistry or immunofluorescence^{c} |
| CR | Negative immunofixation on the serum and urine and |
| | Disappearance of any soft tissue plasmacytomas and <5% |
| | plasma cells in bone marrow^{b} |
| VGPR | Serum and urine M-protein detectable by immunofixation but |
| | not on electrophoresis or 90% or greater reduction in serum |
| | M-protein plus urine M-protein level <100 mg per 24 h |
| PR | ≥50% reduction of serum M-protein and reduction in 24-h |
| | urinary M-protein by ≥90% or to <200 mg per 24 h |
| | If the serum and urine M-protein are unmeasurable,^{d} a ≥50% |
| | decrease in the difference between involved and uninvolved |
| | FLC levels is required in place of the M-protein criteria |
| | If serum and urine M-protein are unmeasurable, and serum free |
| | light assay is also unmeasurable, ≥50% reduction in plasma |
| | cells is required in place of M-protein, provided baseline bone |
| | marrow plasma cell percentage was ≥30% |
| | In addition to the above listed criteria, if present at baseline, a ≥50% |
| | reduction in the size of soft tissue plasmacytomas is also required |
| SD (not | Not meeting criteria for CR, VGPR, PR or progressive disease |
| recommended for | |
| use as an indicator | |
| of response; stability | |
| of disease is best | |
| described by | |
| providing the time | |
| to progression | |
| estimates) | |

Abbreviations: CR, complete response; FLC, free light chain; PR, partial response; SD, stable disease; sCR, stringent complete response; VGPR, very good partial response.
^{a}All response categories require two consecutive assessments made at any time before the institution of any new therapy; all categories also require no known evidence of progressive or new bone lesions if radiographic studies were performed. Radiographic studies are not required to satisfy these response requirements.
^{b}Confirmation with repeat bone marrow biopsy not needed.
^{c}Presence/absence of clonal cells is based upon the κ/λ ratio. An abnormal κ/λ ratio by immunohistochemistry and/or immunofluorescence requires a minimum of 100 plasma cells for analysis. An abnormal ratio reflecting presence of an abnormal clone is κ/λ of >4:1 or <1:2.
^{d}Measurable disease defined by at least one of the following measurements: Bone marrow plasma cells ≥30%; Serum M-protein ≥1 g/dl (≥10 gm/l)[10 g/l]; Urine M-protein ≥200 mg/24 h; Serum FLC assay: Involved FLC level ≥10 mg/dl (≥100 mg/l); provided serum FLC ratio is abnormal.

As used herein, ECOG status refers to Eastern Cooperative Oncology Group (ECOG) Performance Status (Oken M, et al Toxicity and response criteria of the Eastern Cooperative Oncology Group. Am J Clin Oncol 1982; 5(6):649-655), as shown below:

| Score | Description |
|---|---|
| 0 | Fully active, able to carry on all pre-disease performance without |
| | restriction |
| 1 | Restricted in physically strenuous activity but ambulatory and |
| | able to carry out work of a light or sedentary nature, eg, light |
| | housework, office work. |
| 2 | Ambulatory and capable of all self-care but unable to carry out |
| | any work activities. Up and about more than 50% of waking hours. |
| 3 | Capable of only limited self-care, confined to bed or chair more |
| | than 50% of waking hours. |
| 4 | Completely disabled. Cannot carry on any self-care. Totally |
| | confined to bed or chair |
| 5 | Dead |

In certain embodiments, stable disease or lack thereof can be determined by methods known in the art such as evaluation of patient symptoms, physical examination, visualization of the tumor that has been imaged, for example using FDG-PET (fluorodeoxyglucose positron emission tomography), PET/CT (positron emission tomography/computed tomography) scan, MRI (magnetic resonance imaging) of the brain and spine, CSF (cerebrospinal fluid), ophthalmologic exams, vitreal fluid sampling, retinal photograph, bone marrow evaluation and other commonly accepted evaluation modalities.

As used herein and unless otherwise indicated, the terms “co-administration” and “in combination with” include the administration of one or more therapeutic agents (for example, a compound provided herein and another anti-cancer agent or supportive care agent) either simultaneously, concurrently or sequentially with no specific time limits. In one embodiment, the agents are present in the cell or in the patient's body at the same time or exert their biological or therapeutic effect at the same time. In one embodiment, the therapeutic agents are in the same composition or unit dosage form. In another embodiment, the therapeutic agents are in separate compositions or unit dosage forms.

The term “supportive care agent” refers to any substance that treats, prevents or manages an adverse effect from treatment with another therapeutic agent.

As used herein, “induction therapy” refers to the first treatment given for a disease, or the first treatment given with the intent of inducing complete remission in a disease, such as cancer. When used by itself, induction therapy is the one accepted as the best available treatment. If residual cancer is detected, patients are treated with another therapy, termed reinduction. If the patient is in complete remission after induction therapy, then additional consolidation and/or maintenance therapy is given to prolong remission or to potentially cure the patient.

As used herein, “consolidation therapy” refers to the treatment given for a disease after remission is first achieved. For example, consolidation therapy for cancer is the treatment given after the cancer has disappeared after initial therapy. Consolidation therapy may include radiation therapy, stem cell transplant, or treatment with cancer drug therapy. Consolidation therapy is also referred to as intensification therapy and post-remission therapy.

As used herein, “maintenance therapy” refers to the treatment given for a disease after remission or best response is achieved, in order to prevent or delay relapse. Maintenance therapy can include chemotherapy, hormone therapy or targeted therapy.

“Remission” as used herein, is a decrease in or disappearance of signs and symptoms of a cancer, for example, multiple myeloma. In partial remission, some, but not all, signs and symptoms of the cancer have disappeared. In complete remission, all signs and symptoms of the cancer have disappeared, although the cancer still may be in the body.

As used herein “transplant” refers to high-dose therapy with stem cell rescue. Hematopoietic (blood) or bone marrow stem cells are used not as treatment but to rescue the patient after the high-dose therapy, for example high dose chemotherapy and/or radiation. Transplant includes “autologous” stem cell transplant (ASCT), which refers to use of the patients' own stem cells being harvested and used as the replacement cells. In some embodiments, transplant also includes tandem transplant or multiple transplants.

The term “biological therapy” refers to administration of biological therapeutics such as cord blood, stem cells, growth factors and the like.

### B. Compounds

Provided for use in the methods provided herein is the compound 4-amino-2-(2,6-dioxopiperidine-3-yl)isoindoline-1,3-dione (Compound 1):

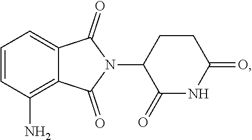 [SMILES: Nc1cccc2c1C(=O)N(C1CCC(=O)NC1=O)C2=O]

or a stereoisomer or mixture of stereoisomers, pharmaceutically acceptable salt, tautomer, prodrug, solvate, hydrate, co-crystal, clathrate, or polymorph thereof. Compound 1 is also known as pomalidomide, or Pom as used herein. In one embodiment, Compound 1 is used in the methods provided herein.

Also provided for use in the methods provided herein is the compound 3-(4-amino-1-oxo-1,3 dihydro-isoindol-2-yl)-piperidine-2,6-dione (Compound 2):

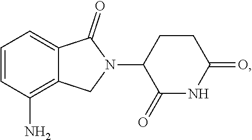 [SMILES: Nc1cccc2c1CN(C1CCC(=O)NC1=O)C2=O]

or a stereoisomer or mixture of stereoisomers, pharmaceutically acceptable salt, tautomer, prodrug, solvate, hydrate, co-crystal, clathrate, or polymorph thereof. Compound 2 is also known as lenalidomide, or Len as used herein. In one embodiment, Compound 2 is used in the methods provided herein.

Also provided for use in the methods provided herein is the compound 2-(2,6-Dioxo-3-piperidinyl)-1H-isoindole-1,3(2H)-dione (Compound 3):

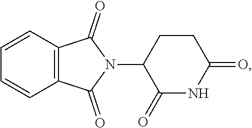 [SMILES: O=C1CCC(N2C(=O)c3ccccc3C2=O)C(=O)N1]

or a stereoisomer or mixture of stereoisomers, pharmaceutically acceptable salt, tautomer, prodrug, solvate, hydrate, co-crystal, clathrate, or polymorph thereof. Compound 3 is also known as thalidomide, or Thal as used herein. In one embodiment, Compound 3 is used in the methods provided herein.

Also provided for use in the methods provided herein is the compound 3-(5-amino-2-methyl-4-oxo-4H-quinazolin-3-yl)-piperidine-2,6-dione (Compound 4):

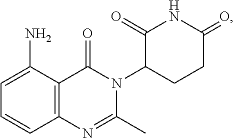 [SMILES: Cc1nc2cccc(N)c2c(=O)n1C1CCC(=O)NC1=O]

or a stereoisomer or mixture of stereoisomers, pharmaceutically acceptable salt, tautomer, prodrug, solvate, hydrate, co-crystal, clathrate, or polymorph thereof. A method for preparing Compound 4 is described in U.S. Pat. No. 7,635,700, which is incorporated herein by reference in its entirety. In one embodiment, Compound 4 is used in the methods provided herein. In one embodiment, a hydrochloride salt of Compound 4 is used in the methods provided herein.

Also provided for use in the methods provided herein is the compound (S)-3-(4-((4-(morpholinomethyl)benzyl)oxy)-1-oxoisoindolin-2-yl)piperidine-2,6-dione (Compound 5):

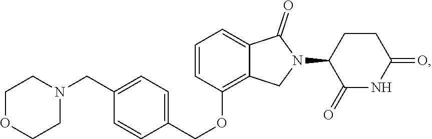 [SMILES: O=C1CC[C@H](N2Cc3c(OCc4ccc(CN5CCOCC5)cc4)cccc3C2=O)C(=O)N1]

or a stereoisomer or mixture of stereoisomers, pharmaceutically acceptable salt, tautomer, prodrug, solvate, hydrate, co-crystal, clathrate, or polymorph thereof. A method for preparing Compound 5 is described in U.S. Pat. No. 8,518,972, which is incorporated herein by reference in its entirety. In one embodiment, Compound 5 is used in the methods provided herein. In one embodiment, a hydrochloride salt of Compound 5 is used in the methods provided herein.

Also provided for use in the methods provided herein is the compound (S)-4-(4-(4-(((2-(2,6-dioxopiperidin-3-yl)-1-oxoisoindolin-4-yl)oxy)methyl)benzyl)piperazin-1-yl)-3-fluorobenzonitrile (Compound 6):

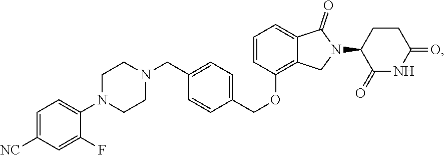 [SMILES: N#Cc1ccc(N2CCN(Cc3ccc(COc4cccc5c4CN([C@H]4CCC(=O)NC4=O)C5=O)cc3)CC2)c(F)c1]

or a stereoisomer or mixture of stereoisomers, pharmaceutically acceptable salt, tautomer, prodrug, solvate, hydrate, co-crystal, clathrate, or polymorph thereof. A method for preparing Compound 6 is described in U.S. Pat. No. 10,357,489, which is incorporated herein by reference in its entirety. In one embodiment, Compound 6 is used in the methods provided herein. In one embodiment, a hydrobromide salt of Compound 6 is used in the methods provided herein.

Also provided for use in the methods provided herein is the compound 2-(4-chlorophenyl)-N-((2-(2,6-dioxopiperidin-3-yl)-1-oxoisoindolin-5-yl)methyl)-2,2-difluoroacetamide (Compound 7):

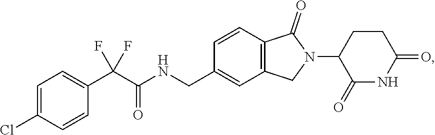 [SMILES: O=C1CCC(N2Cc3cc(CNC(=O)C(F)(F)c4ccc(Cl)cc4)ccc3C2=O)C(=O)N1]

or a stereoisomer or mixture of stereoisomers, pharmaceutically acceptable salt, tautomer, prodrug, solvate, hydrate, co-crystal, clathrate, or polymorph thereof. A method for preparing Compound 7 is described in U.S. Pat. No. 9,499,514, which is incorporated herein by reference in its entirety. In one embodiment, Compound 7 is used in the methods provided herein.

In one embodiment, isotopically enriched analogs of the compounds are used in the methods provided herein.

### C. Second Active Agents

In one embodiment, the second active agent used in the methods provided herein is a polo-like kinase 1 (PLK1) inhibitor. In one embodiment, the PLK1 inhibitor is BI2536, or a stereoisomer, mixture of stereoisomers, tautomer, isotopolog, or pharmaceutically acceptable salt thereof. In one embodiment, the PLK1 inhibitor is BI2536. BI2536 has a chemical name of (R)-4-((8-cyclopentyl-7-ethyl-5-methyl-6-oxo-5,6,7,8-tetrahydropteridin-2-yl)amino)-3-methoxy-N-(1-methylpiperidin-4-yl)benzamide, and has the structure:

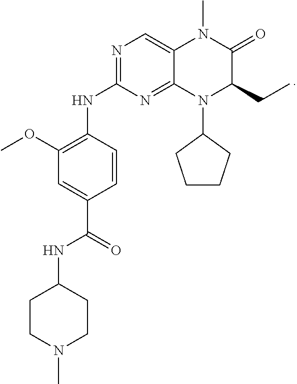 [SMILES: CC[C@@H]1C(=O)N(C)c2cnc(Nc3ccc(C(=O)NC4CCN(C)CC4)cc3OC)nc2N1C1CCCC1]

In one embodiment, the PLK1 inhibitor is volasertib, or a stereoisomer, mixture of stereoisomers, tautomer, isotopolog, or pharmaceutically acceptable salt thereof. In one embodiment, the PLK1 inhibitor is volasertib. Volasertib (also known as BI6727) has the structure:

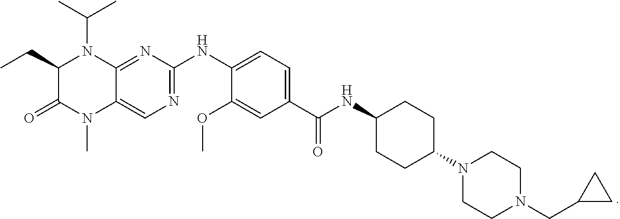 [SMILES: CC[C@@H]1C(=O)N(C)c2cnc(Nc3ccc(C(=O)N[C@H]4CC[C@H](N5CCN(CC6CC6)CC5)CC4)cc3OC)nc2N1C(C)C]

In one embodiment, the PLK1 inhibitor is CYC140, or a stereoisomer, mixture of stereoisomers, tautomer, isotopolog, or pharmaceutically acceptable salt thereof.

In one embodiment, the PLK1 inhibitor is onvansertib, or a tautomer, isotopolog, or pharmaceutically acceptable salt thereof. In one embodiment, the PLK1 inhibitor is onvansertib. Onvansertib (also known as NMS-1286937) has the structure:

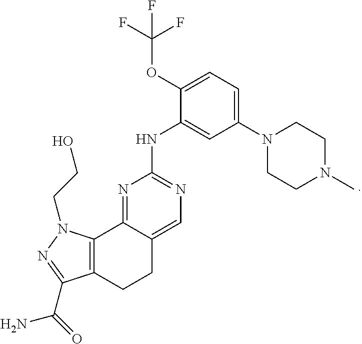 [SMILES: CN1CCN(c2ccc(OC(F)(F)F)c(Nc3ncc4c(n3)-c3c(c(C(N)=O)nn3CCO)CC4)c2)CC1]

In one embodiment, the PLK1 inhibitor is GSK461364, or a stereoisomer, mixture of stereoisomers, tautomer, isotopolog, or pharmaceutically acceptable salt thereof. In one embodiment, the PLK1 inhibitor is GSK461364. GSK461364 has the structure:

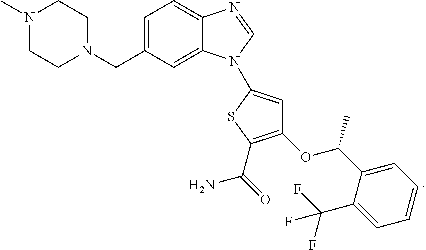 [SMILES: C[C@@H](Oc1cc(-n2cnc3ccc(CN4CCN(C)CC4)cc32)sc1C(N)=O)c1ccccc1C(F)(F)F]

In one embodiment, the PLK1 inhibitor is TAK960, or a tautomer, isotopolog, or pharmaceutically acceptable salt thereof. In one embodiment, the PLK1 inhibitor is TAK960. In one embodiment, the PLK1 inhibitor is a hydrochloride salt of TAK960. TAK960 has the structure:

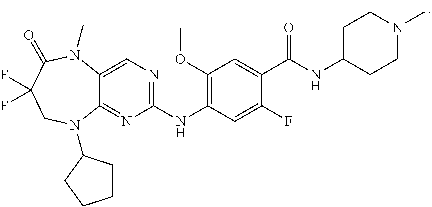 [SMILES: COc1cc(C(=O)NC2CCN(C)CC2)c(F)cc1Nc1ncc2c(n1)N(C1CCCC1)CC(F)(F)C(=O)N2C]

In one embodiment, the second active agent used in the methods provided herein is a bromodomain 4 (BRD4) inhibitor. BRD4 is a member of the BET (bromodomain and extra terminal domain) family. In one embodiment, the BRD4 inhibitor is JQ1, or a stereoisomer, mixture of stereoisomers, tautomer, isotopolog, or pharmaceutically acceptable salt thereof. In one embodiment, the BRD4 inhibitor is JQ1. JQ1 has a chemical name of (S)-tert-butyl 2-(4-(4-chlorophenyl)-2,3,9-trimethyl-6H-thieno[3,2-f][1,2,4]triazolo[4,3-a][1,4]diazepin-6-yl)acetate, and has the structure:

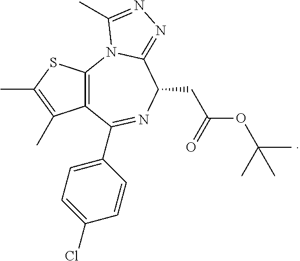 [SMILES: Cc1sc2c(c1C)C(c1ccc(Cl)cc1)=N[C@@H](CC(=O)OC(C)(C)C)c1nnc(C)n1-2]

In one embodiment, the second active agent used in the methods provided herein is a BET inhibitor. In one embodiment, the BET inhibitor is birabresib, or a stereoisomer, mixture of stereoisomers, tautomer, isotopolog, or pharmaceutically acceptable salt thereof. In one embodiment, the BET inhibitor is birabresib. Birabresib (also known as OTX015 or MK-8628) has a chemical name of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide, and has the structure:

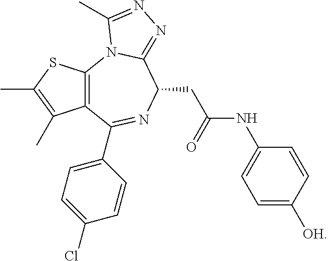 [SMILES: Cc1sc2c(c1C)C(c1ccc(Cl)cc1)=N[C@@H](CC(=O)Nc1ccc(O)cc1)c1nnc(C)n1-2]

In one embodiment, the BET inhibitor is Compound A, or a tautomer, isotopolog, or pharmaceutically acceptable salt thereof. In one embodiment, the BET inhibitor is Compound A. Compound A has a chemical name of 4-[2-(cyclopropylmethoxy)-5-(methanesulfonyl)phenyl]-2-methylisoquinolin-1(2H)-one, and has the structure:

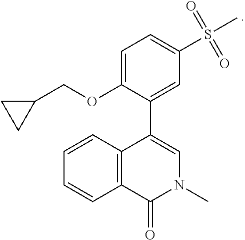 [SMILES: Cn1cc(-c2cc(S(C)(=O)=O)ccc2OCC2CC2)c2ccccc2c1=O]

In one embodiment, the BET inhibitor is BMS-986158, or a stereoisomer, mixture of stereoisomers, tautomer, isotopolog, or pharmaceutically acceptable salt thereof. In one embodiment, the BET inhibitor is BMS-986158. BMS-986158 has the structure:

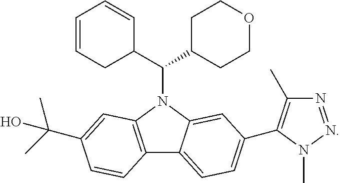 [SMILES: Cc1nnn(C)c1-c1ccc2c3ccc(C(C)(C)O)cc3n([C@H](C3C=CC=CC3)C3CCOCC3)c2c1]

In one embodiment, the BET inhibitor is RO-6870810, or a stereoisomer, mixture of stereoisomers, tautomer, isotopolog, or pharmaceutically acceptable salt thereof. In one embodiment, the BET inhibitor is RO-6870810. RO-6870810 has the structure:

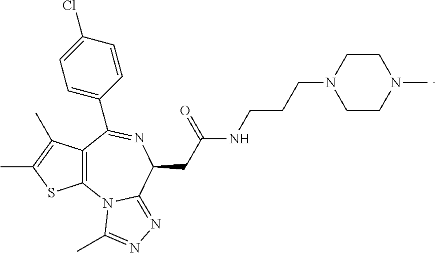

In one embodiment, the BET inhibitor is CPI-0610, or a stereoisomer, mixture of stereoisomers, tautomer, isotopolog, or pharmaceutically acceptable salt thereof. In one embodiment, the BET inhibitor is CPI-0610. CPI-0610 has the structure:

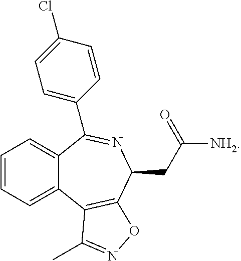 [SMILES: Cc1noc2c1-c1ccccc1C(c1ccc(Cl)cc1)=N[C@H]2CC(N)=O]

In one embodiment, the BET inhibitor is molibresib, or a stereoisomer, mixture of stereoisomers, tautomer, isotopolog, or pharmaceutically acceptable salt thereof. In one embodiment, the BET inhibitor is molibresib. Molibresib (also known as GSK-525762) has the structure:

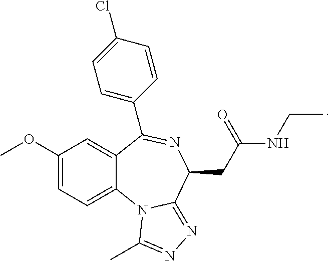 [SMILES: CCNC(=O)C[C@@H]1N=C(c2ccc(Cl)cc2)c2cc(OC)ccc2-n2c(C)nnc21]

In one embodiment, the second active agent used in the methods provided herein is a serine/threonine-protein kinase (NEK2) inhibitor. In one embodiment, the NEK2 inhibitor is JH295, or a tautomer, isotopolog, or pharmaceutically acceptable salt thereof. In one embodiment, the NEK2 inhibitor is JH295. JH295 has a chemical name of (Z)—N-(3-((2-ethyl-4-methyl-1H-imidazol-5-yl)methylene)-2-oxoindolin-5-yl)propiolamide, and has the structure:

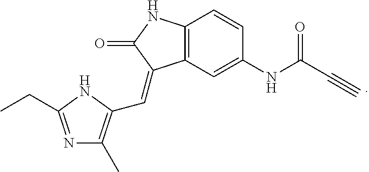 [SMILES: C#CC(=O)Nc1ccc2c(c1)/C(=C/c1[nH]c(CC)nc1C)C(=O)N2]

In one embodiment, the NEK2 inhibitor is rac-CCT 250863, or a stereoisomer, mixture of stereoisomers, tautomer, isotopolog, or pharmaceutically acceptable salt thereof. In one embodiment, the NEK2 inhibitor is rac-CCT 250863. Rac-CCT 250863 has a chemical name of 4-[2-amino-5-[4-[(dimethylamino)methyl]-2-thienyl]-3-pyridinyl]-2-[[(2Z)-4,4,4-trifluoro-1-methyl-2-buten-1-yl]oxy]benzamide, and has the structure:

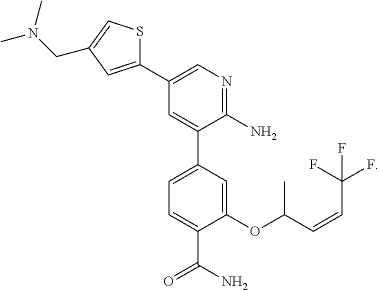 [SMILES: CC(/C=C\C(F)(F)F)Oc1cc(-c2cc(-c3cc(CN(C)C)cs3)cnc2N)ccc1C(N)=O]

In one embodiment, the second active agent used in the methods provided herein is an Aurora kinase B (AURKB) inhibitor. In one embodiment, the AURKB inhibitor is barasertib (also known as AZD1152) or AZD1152-HQPA, or a tautomer, isotopolog, or pharmaceutically acceptable salt thereof. In one embodiment, the AURKB inhibitor is barasertib. In one embodiment, the AURKB inhibitor is AZD1152-HQPA. AZD1152-HQPA (also known as AZD2811) has a chemical name of 2-(3-((7-(3-(ethyl(2-hydroxyethyl)amino)propoxy)quinazolin-4-yl)amino)-1H-pyrazol-5-yl)-N-(3-fluorophenyl)acetamide, and has the structure:

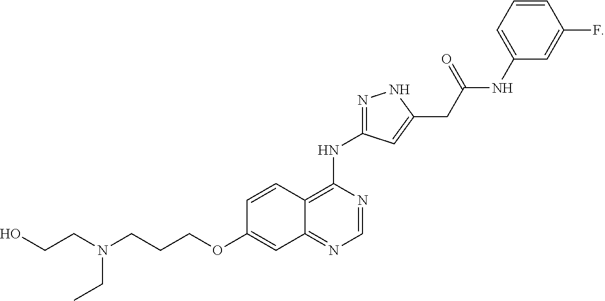 [SMILES: CCN(CCO)CCCOc1ccc2c(Nc3cc(CC(=O)Nc4cccc(F)c4)[nH]n3)ncnc2c1]

Barasertib is a dihydrogen phosphate prodrug of AZD1152-HQPA, and has the structure:

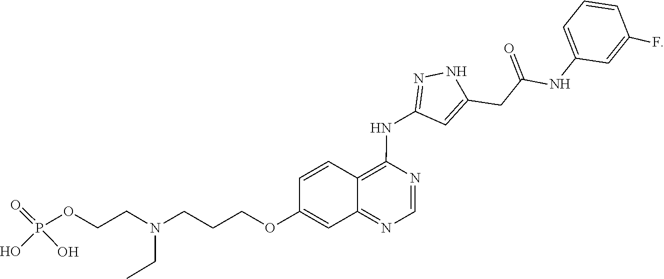 [SMILES: CCN(CCCOc1ccc2c(Nc3cc(CC(=O)Nc4cccc(F)c4)[nH]n3)ncnc2c1)CCOP(=O)(O)O]

In one embodiment, the AURKB inhibitor is alisertib, or a tautomer, isotopolog, or pharmaceutically acceptable salt thereof. In one embodiment, the Aurora A kinase inhibitor is alisertib. Alisertib has a chemical name of 4-((9-chloro-7-(2-fluoro-6-methoxyphenyl)-5H-benzo[c]pyrimido[4,5-e]azepin-2-yl)amino)-2-methoxybenzoic acid, and has the structure:

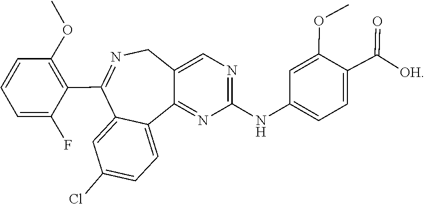 [SMILES: COc1cc(Nc2ncc3c(n2)-c2ccc(Cl)cc2C(c2c(F)cccc2OC)=NC3)ccc1C(=O)O]

In one embodiment, the AURKB inhibitor is danusertib, or a stereoisomer, mixture of stereoisomers, tautomer, isotopolog, or pharmaceutically acceptable salt thereof. In one embodiment, the AURKB inhibitor is danusertib. Danusertib (also known as PHA-739358) has the structure:

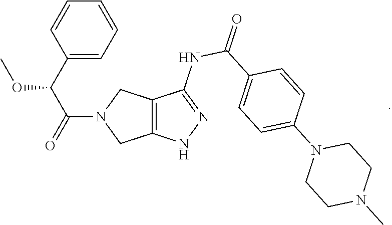

In one embodiment, the AURKB inhibitor is AT9283, or a tautomer, isotopolog, or pharmaceutically acceptable salt thereof. In one embodiment, the Aurora A kinase inhibitor is AT9283. AT9283 has the structure:

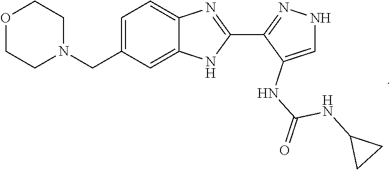 [SMILES: O=C(Nc1c[nH]nc1-c1nc2ccc(CN3CCOCC3)cc2[nH]1)NC1CC1]

In one embodiment, the AURKB inhibitor is PF-03814735, or a stereoisomer, mixture of stereoisomers, tautomer, isotopolog, or pharmaceutically acceptable salt thereof. In one embodiment, the AURKB inhibitor is PF-03814735. PF-03814735 has the structure:

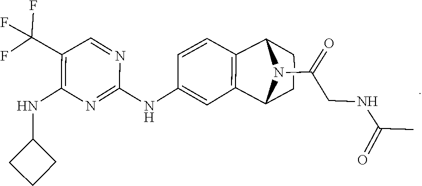 [SMILES: CC[C@@H]1c2cc(Nc3ncc(C(F)(F)F)c(NC4CCC4)n3)ccc2[C@H](CC)N1C(=O)CNC(C)=O]

In one embodiment, the AURKB inhibitor is AMG900, or a tautomer, isotopolog, or pharmaceutically acceptable salt thereof. In one embodiment, the Aurora A kinase inhibitor is AMG900. AMG900 has the structure:

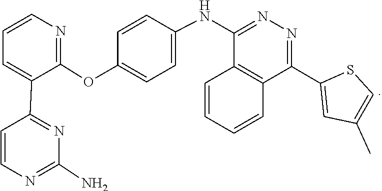 [SMILES: Cc1csc(-c2nnc(Nc3ccc(Oc4ncccc4-c4ccnc(N)n4)cc3)c3ccccc23)c1]

In one embodiment, the AURKB inhibitor is tozasertib, or a tautomer, isotopolog, or pharmaceutically acceptable salt thereof. In one embodiment, the Aurora A kinase inhibitor is tozasertib. Tozasertib (also known as VX-680 or MK-0457) has the structure:

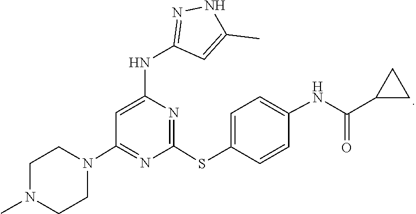 [SMILES: Cc1cc(Nc2cc(N3CCN(C)CC3)nc(Sc3ccc(NC(=O)C4CC4)cc3)n2)n[nH]1]

In one embodiment, the AURKB inhibitor is ZM447439, or a tautomer, isotopolog, or pharmaceutically acceptable salt thereof. In one embodiment, the Aurora A kinase inhibitor is ZM447439. ZM447439 has the structure:

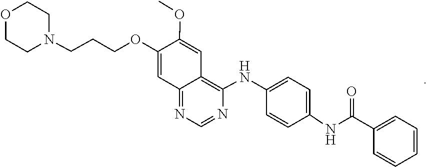 [SMILES: COc1cc2c(Nc3ccc(NC(=O)c4ccccc4)cc3)ncnc2cc1OCCCN1CCOCC1]

In one embodiment, the AURKB inhibitor is MLN8054, or a tautomer, isotopolog, or pharmaceutically acceptable salt thereof. In one embodiment, the Aurora A kinase inhibitor is MLN8054. MLN8054 has the structure:

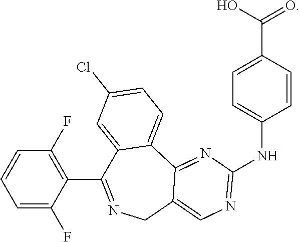 [SMILES: O=C(O)c1ccc(Nc2ncc3c(n2)-c2ccc(Cl)cc2C(c2c(F)cccc2F)=NC3)cc1]

In one embodiment, the AURKB inhibitor is hesperadin, or a tautomer, isotopolog, or pharmaceutically acceptable salt thereof. In one embodiment, the Aurora A kinase inhibitor is hesperadin. In one embodiment, the Aurora A kinase inhibitor is a hydrochloride salt of hesperadin. Hesperadin has the structure:

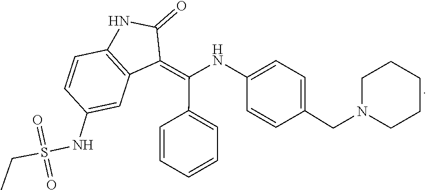 [SMILES: CCS(=O)(=O)Nc1ccc2c(c1)/C(=C(/Nc1ccc(CN3CCCCC3)cc1)c1ccccc1)C(=O)N2]

In one embodiment, the AURKB inhibitor is SNS-314, or a tautomer, isotopolog, or pharmaceutically acceptable salt thereof. In one embodiment, the Aurora A kinase inhibitor is SNS-314. In one embodiment, the Aurora A kinase inhibitor is a mesylate salt of SNS-314. SNS-314 has the structure:

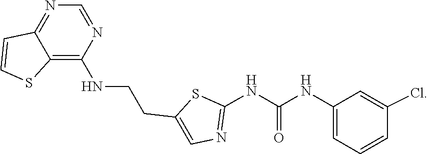 [SMILES: O=C(Nc1cccc(Cl)c1)Nc1ncc(CCNc2ncnc3ccsc23)s1]

In one embodiment, the AURKB inhibitor is PHA-680632, or a tautomer, isotopolog, or pharmaceutically acceptable salt thereof. In one embodiment, the Aurora A kinase inhibitor is PHA-680632. PHA-680632 has the structure:

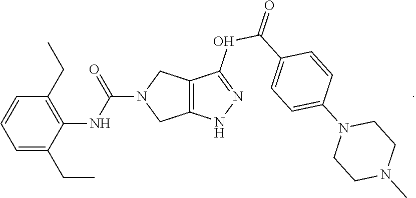 [SMILES: CCc1cccc(CC)c1NC(=O)N1Cc2[nH]nc(OC(=O)c3ccc(N4CCN(C)CC4)cc3)c2C1]

In one embodiment, the AURKB inhibitor is CYC116, or a tautomer, isotopolog, or pharmaceutically acceptable salt thereof. In one embodiment, the Aurora A kinase inhibitor is CYC116. CYC116 has the structure:

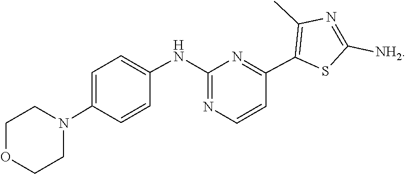 [SMILES: Cc1nc(N)sc1-c1ccnc(Nc2ccc(N3CCOCC3)cc2)n1]

In one embodiment, the AURKB inhibitor is GSK1070916, or a tautomer, isotopolog, or pharmaceutically acceptable salt thereof. In one embodiment, the Aurora A kinase inhibitor is GSK1070916. GSK1070916 has the structure:

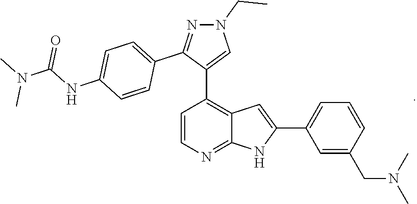

In one embodiment, the AURKB inhibitor is TAK-901, or a tautomer, isotopolog, or pharmaceutically acceptable salt thereof. In one embodiment, the Aurora A kinase inhibitor is TAK-901. TAK-901 has the structure:

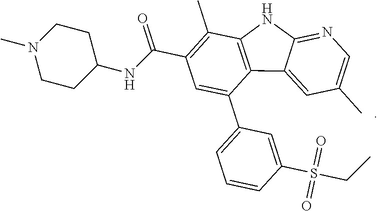 [SMILES: CCS(=O)(=O)c1cccc(-c2cc(C(=O)NC3CCN(C)CC3)c(C)c3[nH]c4ncc(C)cc4c23)c1]

In one embodiment, the AURKB inhibitor is CCT137690, or a tautomer, isotopolog, or pharmaceutically acceptable salt thereof. In one embodiment, the Aurora A kinase inhibitor is CCT137690. CCT137690 has the structure:

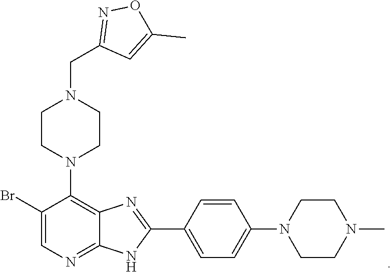 [SMILES: Cc1cc(CN2CCN(c3c(Br)cnc4[nH]c(-c5ccc(N6CCN(C)CC6)cc5)nc34)CC2)no1]

In one embodiment, the second active agent used in the methods provided herein is a mitogen-activated extracellular signal-regulated kinase (MEK) inhibitor. In one embodiment, the MEK inhibitor interrupts the function of the RAF/RAS/MEK signal transduction cascade. In one embodiment, the MEK inhibitor is trametinib, trametinib dimethyl sulfoxide, cobimetinib, binimetinib, or selumetinib, or a stereoisomer, mixture of stereoisomers, tautomer, isotopolog, or pharmaceutically acceptable salt thereof. In one embodiment, the MEK inhibitor is trametinib or trametinib dimethyl sulfoxide, or a stereoisomer, mixture of stereoisomers, tautomer, isotopolog, or pharmaceutically acceptable salt thereof. In one embodiment, the MEK inhibitor is trametinib. In one embodiment, the MEK inhibitor is trametinib dimethyl sulfoxide. In one embodiment, the MEK inhibitor is cobimetinib. In one embodiment, the MEK inhibitor is binimetinib. In one embodiment, the MEK inhibitor is selumetinib. Trametinib dimethyl sulfoxide has a chemical name of N-[3-[3-cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]-acetamide, compound with dimethyl sulfoxide (1:1). Trametinib dimethyl sulfoxide has the structure:

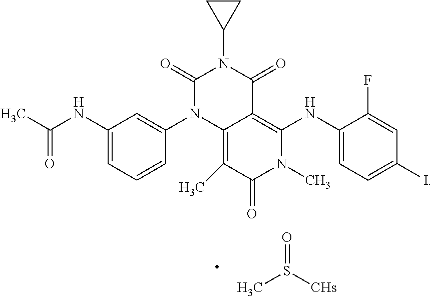

In one embodiment, the second active agent used in the methods provided herein is a PHD Finger Protein 19 (PIF19) inhibitor.

In one embodiment, the second active agent used in the methods provided herein is a Bruton's tyrosine kinase (BTK) inhibitor. In one embodiment, the BTK inhibitor is ibrutinib, or acalabrutinib, or a stereoisomer, mixture of stereoisomers, tautomer, isotopolog, or pharmaceutically acceptable salt thereof. In one embodiment, the BTK inhibitor is ibrutinib, or a stereoisomer, mixture of stereoisomers, tautomer, isotopolog, or pharmaceutically acceptable salt thereof. In one embodiment, the BTK inhibitor is ibrutinib. In one embodiment, the BTK inhibitor is acalabrutinib. Ibrutinib has a chemical name of 1-[(3R)-3-[4-amino-3-(4-phenoxyphenyl)-1Hpyrazolo[3,4-d]pyrimidin-1-yl]-1-piperidinyl]-2-propen-1-one, and has the structure:

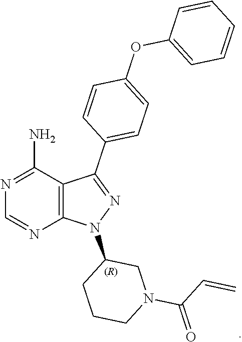

In one embodiment, the second active agent used in the methods provided herein is a mammalian target of rapamycin (mTOR) inhibitor. In one embodiment, the mTOR inhibitor is rapamycin or an analog thereof (also termed rapalog). In one embodiment, the mTOR inhibitor is everolimus, or a stereoisomer, mixture of stereoisomers, tautomer, isotopolog, or pharmaceutically acceptable salt thereof. In one embodiment, the mTOR inhibitor is everolimus. Everolimus has a chemical name of 40-O-(2-hydroxyethyl)-rapamycin, and has the structure:

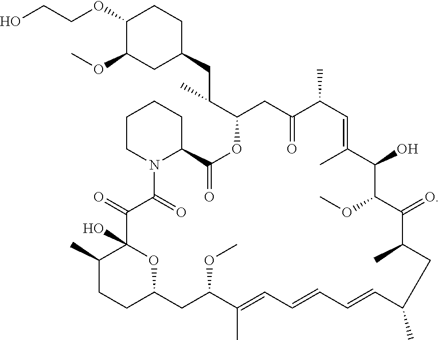 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In one embodiment, the second active agent used in the methods provided herein is a proviral integration site for Moloney murine leukemia kinase (PIM) inhibitor. In one embodiment, the PIM inhibitor is a pan-PIM inhibitor. In one embodiment, the PIM inhibitor is LGH-447, AZD1208, SGI-1776, or TP-3654, or a stereoisomer, mixture of stereoisomers, tautomer, isotopolog, or pharmaceutically acceptable salt thereof. In one embodiment, the PIM inhibitor is LGH-447, or a stereoisomer, mixture of stereoisomers, tautomer, isotopolog, or pharmaceutically acceptable salt thereof. In one embodiment, the PIM inhibitor is LGH-447. In one embodiment, the PIM inhibitor is a pharmaceutically acceptable salt of LGH-447. In one embodiment, the PIM inhibitor is a hydrochloride salt of LGH-447. In one embodiment, the hydrochloride salt of LGH-447 is a di-hydrochloride salt. In one embodiment, the hydrochloride salt of LGH-447 is a mono-hydrochloride salt. In one embodiment, the PIM inhibitor is AZD1208. In one embodiment, the PIM inhibitor is SGI-1776. In one embodiment, the PIM inhibitor is TP-3654. LGH-447 has a chemical name of N-[4-[(1R,3S,5S)-3-amino-5-methylcyclohexyl]-3-pyridinyl]-6-(2,6-difluorophenyl)-5-fluoro-2-pyridinecarboxamide, and has the structure:

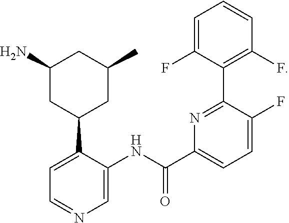 [SMILES: C[C@@H]1C[C@H](N)C[C@H](c2ccncc2NC(=O)c2ccc(F)c(-c3c(F)cccc3F)n2)C1]

In one embodiment, the second active agent used in the methods provided herein is an insulin-like growth factor 1 receptor (IGF-1R) inhibitor. In one embodiment, the IGF-1R inhibitor is linsitinib, or a stereoisomer, mixture of stereoisomers, tautomer, isotopolog, or pharmaceutically acceptable salt thereof. In one embodiment, the IGF-1R inhibitor is linsitinib. Linsitinib has a chemical name of cis-3-[8-amino-1-(2-phenyl-7-quinolinyl)imidazo[1,5-a]pyrazin-3-yl]-1-methylcyclobutanol, and has the structure:

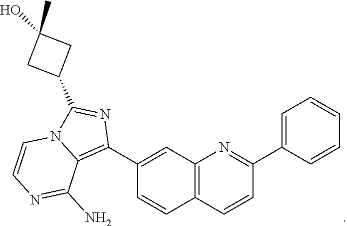 [SMILES: C[C@]1(O)C[C@@H](c2nc(-c3ccc4ccc(-c5ccccc5)nc4c3)c3c(N)nccn32)C1]

In one embodiment, the second active agent used in the methods provided herein is an exportin 1 (XPO1) inhibitor. In one embodiment, the XPO1 inhibitor is selinexor, or a stereoisomer, mixture of stereoisomers, tautomer, isotopolog, or pharmaceutically acceptable salt thereof. In one embodiment, the XPO1 inhibitor is selinexor. Selinexor has a chemical name of (2Z)-3-{3-[3,5-bis(trifluoromethyl)phenyl]-1H-1,2,4-triazol-1-yl}-N′-(pyrazin-2-yl)prop-2-enehydrazide, and has the structure:

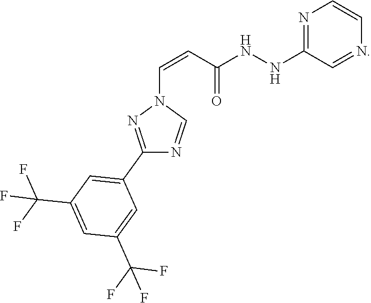 [SMILES: O=C(/C=C\n1cnc(-c2cc(C(F)(F)F)cc(C(F)(F)F)c2)n1)NNc1cnccn1]

In one embodiment, the second active agent used in the methods provided herein is a disruptor of telomeric silencing 1-like (DOT1L) inhibitor. In one embodiment, the DOT1L inhibitor is SGC0946, or pinometostat, or a stereoisomer, mixture of stereoisomers, tautomer, isotopolog, or pharmaceutically acceptable salt thereof. In one embodiment, the DOT1L inhibitor is SGC0946, or a stereoisomer, mixture of stereoisomers, tautomer, isotopolog, or pharmaceutically acceptable salt thereof. In one embodiment, the DOT1L inhibitor is SGC0946. SGC0946 has a chemical name of 5 bromo-7-[5-deoxy-5-[[3-[[[[4-(1,1-dimethylethyl)phenyl]amino]carbonyl]amino]propyl](1-methylethyl)amino]-β-D-ribofuranosyl]-7H-pyrrolo[2,3-d]pyrimidin-4-amine, and has the structure:

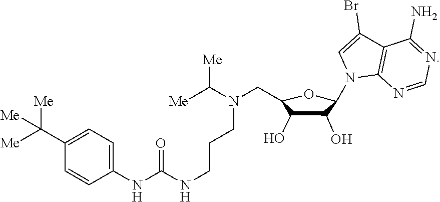

In one embodiment, the DOT1L inhibitor is pinometostat, or a stereoisomer, mixture of stereoisomers, tautomer, isotopolog, or pharmaceutically acceptable salt thereof. In one embodiment, the DOT1L inhibitor is pinometostat. Pinometostat (also known as EPZ-5676) has a chemical name of (2R,3R,4S,5R)-2-(6-amino-9H-purin-9-yl)-5-((((1r,3S)-3-(2-(5-(tert-butyl)-1H-benzo[d]imidazol-2-yl)ethyl)cyclobutyl)(isopropyl)amino)methyl)tetrahydrofuran-3,4-diol, and has the structure:

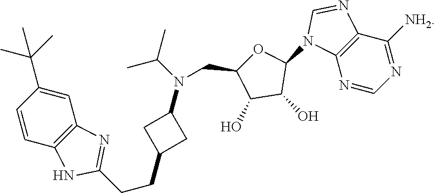

In one embodiment, the second active agent used in the methods provided herein is an enhancer of zeste homolog 2 (EZH2) inhibitor. In one embodiment, the EZH2 inhibitor is tazemetostat, 3-deazaneplanocin A (DZNep), EPZ005687, Ell, GSK126, UNC1999, CPI-1205, or sinefungin, or a stereoisomer, mixture of stereoisomers, tautomer, isotopolog, or pharmaceutically acceptable salt thereof. In one embodiment, the EZH2 inhibitor is tazemetostat, or a stereoisomer, mixture of stereoisomers, tautomer, isotopolog, or pharmaceutically acceptable salt thereof. In one embodiment, the EZH2 inhibitor is tazemetostat. In one embodiment, the EZH2 inhibitor is 3-deazaneplanocin A. In one embodiment, the EZH2 inhibitor is EPZ005687. In one embodiment, the EZH2 inhibitor is EI1. In one embodiment, the EZH2 inhibitor is GSK126. In one embodiment, the EZH2 inhibitor is sinefungin. Tazemetostat (also known as EPZ-6438) has a chemical name of N-[(1,2-dihydro-4,6-dimethyl-2-oxo-3-pyridinyl)methyl]-5-[ethyl(tetrahydro-2H-pyran-4-yl)amino]-4-methyl-4′-(4-morpholinylmethyl)-[1,1′-biphenyl]-3-carboxamide, and has the structure:

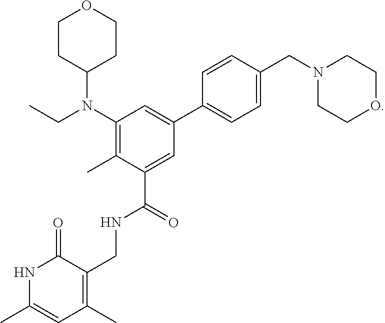 [SMILES: CCN(c1cc(-c2ccc(CN3CCOCC3)cc2)cc(C(=O)NCc2c(C)cc(C)[nH]c2=O)c1C)C1CCOCC1]

In one embodiment, the EZH2 inhibitor is UNC1999, or a tautomer, isotopolog, or pharmaceutically acceptable salt thereof. In one embodiment, the EZH2 inhibitor is UNC1999. UNC1999 has a chemical name of 1-Isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide, and has the structure:

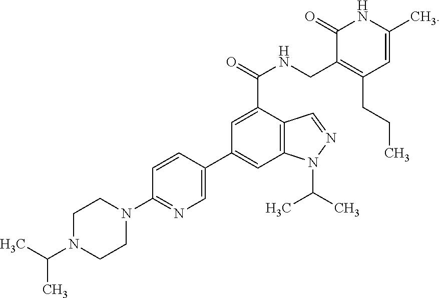 [SMILES: CCCc1cc(C)[nH]c(=O)c1CNC(=O)c1cc(-c2ccc(N3CCN(C(C)C)CC3)nc2)cc2c1cnn2C(C)C]

In one embodiment, the EZH2 inhibitor is CPI-1205, or a stereoisomer, mixture of stereoisomers, tautomer, isotopolog, or pharmaceutically acceptable salt thereof. In one embodiment, the EZH2 inhibitor is CPI-1205. CPI-1205 has a chemical name of (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, and has the structure:

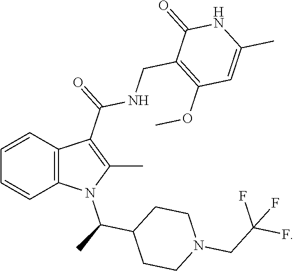 [SMILES: COc1cc(C)[nH]c(=O)c1CNC(=O)c1c(C)n([C@H](C)C2CCN(CC(F)(F)F)CC2)c2ccccc12]

In one embodiment, the second active agent used in the methods provided herein is a Janus kinase 2 (JAK2) inhibitor. In one embodiment, the JAK2 inhibitor is fedratinib, ruxolitinib, baricitinib, gandotinib, lestaurtinib, momelotinib, or pacritinib, or a stereoisomer, mixture of stereoisomers, tautomer, isotopolog, or pharmaceutically acceptable salt thereof. In one embodiment, the JAK2 inhibitor is fedratinib, or a tautomer, isotopolog, or pharmaceutically acceptable salt thereof. In one embodiment, the JAK2 inhibitor is fedratinib. In one embodiment, the JAK2 inhibitor is ruxolitinib. In one embodiment, the JAK2 inhibitor is baricitinib. In one embodiment, the JAK2 inhibitor is gandotinib. In one embodiment, the JAK2 inhibitor is lestaurtinib. In one embodiment, the JAK2 inhibitor is momelotinib. In one embodiment, the JAK2 inhibitor is pacritinib. Fedratinib has a chemical name of N-tert-butyl-3-[(5-methyl-2-{4-[2-(pyrrolidin-1-yl)ethoxy]anilino}pyrimidin-4-yl)amino]benzenesulfonamide, and has the structure:

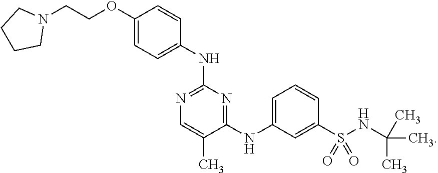 [SMILES: Cc1cnc(Nc2ccc(OCCN3CCCC3)cc2)nc1Nc1cccc(S(=O)(=O)NC(C)(C)C)c1]

In one embodiment, the second active agent used in the methods provided herein is a survivin (also called baculoviral inhibitor of apoptosis repeat-containing 5 or BIRC5) inhibitor. In one embodiment, the BIRC5 inhibitor is YM155, or a tautomer, isotopolog, or pharmaceutically acceptable salt thereof. In one embodiment, the BIRC5 inhibitor is YM155. YM155 has a chemical name of 1-(2-methoxyethyl)-2-methyl-4,9-dioxo-3-(pyrazin-2-ylmethyl)-4,9-dihydro-1H-naphtho[2,3-d]imidazol-3-ium bromide, and has the structure:

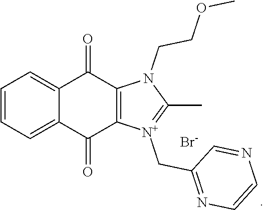 [SMILES: COCCn1c2c([n+](Cc3cnccn3)c1C)C(=O)c1ccccc1C2=O ; [Br-]]

In one embodiment, the second active agent used in the methods provided herein is a DNA methyltransferase inhibitor. In one embodiment, the DNA methyltransferase inhibitor is azacitidine, or a stereoisomer, mixture of stereoisomers, tautomer, isotopolog, or pharmaceutically acceptable salt thereof. In one embodiment, the hypomethylating agent is azacitidine. Azacitidine (also known as azacytidine or 5-azacytidine) has a chemical name of 4-amino-1-β-D-ribofuranosyl-1,3,5-triazin-2(1H)-one, and has the structure:

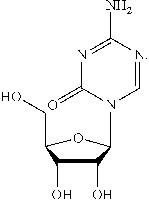

### D. Methods of Use

In one embodiment, provided herein is a method of treating cancer, which comprises administering to a patient in need thereof a therapeutically effective amount of Compound 1, or a stereoisomer or mixture of stereoisomers, pharmaceutically acceptable salt, tautomer, prodrug, solvate, hydrate, co-crystal, clathrate, or polymorph thereof, in combination with a second agent, wherein the second agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHIF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, provided herein is a method of treating cancer, which comprises administering to a patient in need thereof a therapeutically effective amount of Compound 2, or a stereoisomer or mixture of stereoisomers, pharmaceutically acceptable salt, tautomer, prodrug, solvate, hydrate, co-crystal, clathrate, or polymorph thereof, in combination with a second agent, wherein the second agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHIF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, provided herein is a method of treating cancer, which comprises administering to a patient in need thereof a therapeutically effective amount of Compound 3, or a stereoisomer or mixture of stereoisomers, pharmaceutically acceptable salt, tautomer, prodrug, solvate, hydrate, co-crystal, clathrate, or polymorph thereof, in combination with a second agent, wherein the second agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHIF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, provided herein is a method of treating cancer, which comprises administering to a patient in need thereof a therapeutically effective amount of Compound 4, or a stereoisomer or mixture of stereoisomers, pharmaceutically acceptable salt, tautomer, prodrug, solvate, hydrate, co-crystal, clathrate, or polymorph thereof, in combination with a second agent, wherein the second agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHIF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, provided herein is a method of treating cancer, which comprises administering to a patient in need thereof a therapeutically effective amount of Compound 5, or a stereoisomer or mixture of stereoisomers, pharmaceutically acceptable salt, tautomer, prodrug, solvate, hydrate, co-crystal, clathrate, or polymorph thereof, in combination with a second agent, wherein the second agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHIF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, provided herein is a method of treating cancer, which comprises administering to a patient in need thereof a therapeutically effective amount of Compound 6, or a stereoisomer or mixture of stereoisomers, pharmaceutically acceptable salt, tautomer, prodrug, solvate, hydrate, co-crystal, clathrate, or polymorph thereof, in combination with a second agent, wherein the second agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHIF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, provided herein is a method of treating cancer, which comprises administering to a patient in need thereof a therapeutically effective amount of Compound 7, or a stereoisomer or mixture of stereoisomers, pharmaceutically acceptable salt, tautomer, prodrug, solvate, hydrate, co-crystal, clathrate, or polymorph thereof, in combination with a second agent, wherein the second agent is one or more of a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, the cancer is a hematological malignancy.

In one embodiment, the cancer is leukemia. In one embodiment, the cancer is acute myeloid leukemia. In one embodiment, the acute myeloid leukemia is B-cell acute myeloid leukemia. In one embodiment, the cancer is acute lymphocytic leukemia. In one embodiment, the cancer is chronic lymphocytic leukemia/small lymphocytic lymphoma.

In one embodiment, the cancer is a B-cell malignancy.

In one embodiment, the cancer is lymphoma. In one embodiment, the cancer is non-Hodgkin's lymphoma. In one embodiment, the cancer is diffuse large B-cell lymphoma (DLBCL). In one embodiment, the cancer is mantle cell lymphoma (MCL). In one embodiment, the cancer is marginal zone lymphoma (MZL). In one embodiment, the marginal zone lymphoma is splenic marginal zone lymphoma (SMZL). In one embodiment, the cancer is indolent follicular cell lymphoma (iFCL). In one embodiment, the cancer is Burkitt lymphoma.

In one embodiment, the cancer is T-cell lymphoma. In one embodiment, the T-cell lymphoma is anaplastic large cell lymphoma (ALCL). In one embodiment, the T-cell lymphoma is Sezary Syndrome.

In one embodiment, the cancer is Hodgkin's lymphoma.

In one embodiment, the cancer is myelodysplastic syndromes.

In one embodiment, the cancer is myeloma. In one embodiment, the cancer is multiple myeloma. In one embodiment, the multiple myeloma is plasma cell leukemia (PCL).

In one embodiment, the multiple myeloma is newly diagnosed multiple myeloma.

In one embodiment, the multiple myeloma is relapsed or refractory. In one embodiment, the multiple myeloma is refractory to lenalidomide. In one embodiment, the multiple myeloma is refractory to pomalidomide. In one embodiment, the multiple myeloma is refractory to pomalidomide when used in combination with a proteasome inhibitor. In one embodiment, the proteasome inhibitor is selected from bortezomib, carfilzomib, and ixazomib. In one embodiment, the multiple myeloma is refractory to pomalidomide when used in combination with an inflammatory steroid. In one embodiment, the inflammatory steroid is selected from dexamethasone or prednisone. In one embodiment, the multiple myeloma is refractory to pomalidomide when used in combination with a CD38 directed monoclonal antibody.

In one embodiment, provided herein are methods for achieving a complete response, partial response, or stable disease in a patient, comprising administering to a patient having a cancer provided herein a therapeutically effective amount of a compound provided herein in combination with a second active agent provided herein.

In one embodiment, also provided herein are methods for inducing a therapeutic response assessed with the International Uniform Response Criteria for Multiple Myeloma (IURC) (see Durie B G M, Harousseau J-L, Miguel J S, et al. International uniform response criteria for multiple myeloma. Leukemia, 2006; (10) 10: 1-7) of a patient, comprising administering to a patient having multiple myeloma an effective amount of a therapeutically effective amount of a compound provided herein in combination with a second active agent provided herein.

In another embodiment, provided herein are methods for achieving a stringent complete response, complete response, or very good partial response, as determined by the International Uniform Response Criteria for Multiple Myeloma (IURC) in a patient, comprising administering to a patient having multiple myeloma an effective amount of a therapeutically effective amount of a compound provided herein in combination with a second active agent provided herein.

In another embodiment, provided herein are methods for achieving an increase in overall survival, progression-free survival, event-free survival, time to progression, or disease-free survival in a patient, comprising administering to a patient having multiple myeloma an effective amount of a therapeutically effective amount of a compound provided herein in combination with a second active agent provided herein.

In on embodiment, provided herein is a method of identifying a subject having a hematological cancer who is likely to be responsive to a treatment compound in combination with a second agent, or predicting the responsiveness of a subject having a hematological cancer to a treatment compound in combination with a second agent, comprising:
a. obtaining a sample from the subject;
b. determining a biomarker level in the sample;
c. diagnosing the subject as being likely to be responsive to the treatment compound in combination with the second agent if the biomarker level is an altered level relative to a reference level of the biomarker.

In one embodiment, provided herein is a method of selectively treating a hematological cancer in a subject having a hematological cancer, comprising:
a. obtaining a sample from the subject;
b. determining a biomarker level in the sample;
c. diagnosing the subject as being likely to be responsive to the treatment compound in combination with the second agent if the biomarker level is an altered level relative to a reference level of the biomarker; and
d. administering a therapeutically effective amount of the treatment compound in combination with the second agent to the subject diagnosed as being likely to be responsive to the treatment compound in combination with a second agent.

In one embodiment, the biomarker is expression of a gene or a combination of genes selected from: BRD4, PLK1, AURKB, PHF19, NEK2, MEK, BTK, MTOR, PIM, IGF-1R, XPO1, DOT1L, EZH2, JAK2, and BIRC5.

In one embodiment, the altered level is an increased level relative to a reference level of the biomarker. In one embodiment, the altered level is a decreased level relative to a reference level of the biomarker.

In one embodiment, the treatment compound is a compound provided herein (e.g., Compound 1, Compound 2, Compound 3, Compound 4, Compound 5, Compound 6, or Compound 7, or a stereoisomer or mixture of stereoisomers, pharmaceutically acceptable salt, tautomer, prodrug, solvate, hydrate, co-crystal, clathrate, or polymorph thereof).

In one embodiment, the second agent is a second agent provided herein: a PLK1 inhibitor (e.g., BI2536), a BRD4 inhibitor (e.g., JQ1), a BET inhibitor (e.g., Compound A), an NEK2 inhibitor (e.g., JH295), an AURKB inhibitor (e.g., AZD1152), an MEK inhibitor (e.g., trametinib), a PHF19 inhibitor, a BTK inhibitor (e.g., ibrutinib), an mTOR inhibitor (e.g., everolimus), a PIM inhibitor (e.g., LGH-447), an IGF-1R inhibitor (e.g., linsitinib), an XPO1 inhibitor (e.g., selinexor), a DOT1L inhibitor (e.g., SGC0946 or pinometostat), an EZH2 inhibitor (e.g., tazemetostat, UNC1999, or CPI-1205), a JAK2 inhibitor (e.g., fedratinib), a BIRC5 inhibitor (e.g., YM155), or a DNA methyltransferase inhibitor (e.g., azacitidine).

In one embodiment, the biomarker is a gene for PLK1, the treatment compound is Compound 5, or a stereoisomer or mixture of stereoisomers, pharmaceutically acceptable salt, tautomer, prodrug, solvate, hydrate, co-crystal, clathrate, or polymorph thereof, and the second agent is a PLK1 inhibitor.

In one embodiment, the biomarker is a gene for PLK1, the treatment compound is Compound 6, or a stereoisomer or mixture of stereoisomers, pharmaceutically acceptable salt, tautomer, prodrug, solvate, hydrate, co-crystal, clathrate, or polymorph thereof, and the second agent is a PLK1 inhibitor.

In one embodiment, the biomarker is a gene for BRD4, the treatment compound is Compound 5, or a stereoisomer or mixture of stereoisomers, pharmaceutically acceptable salt, tautomer, prodrug, solvate, hydrate, co-crystal, clathrate, or polymorph thereof, and the second agent is a BRD4 inhibitor.

In one embodiment, the biomarker is a gene for BRD4, the treatment compound is Compound 6, or a stereoisomer or mixture of stereoisomers, pharmaceutically acceptable salt, tautomer, prodrug, solvate, hydrate, co-crystal, clathrate, or polymorph thereof, and the second agent is a BRD4 inhibitor.

In one embodiment, the biomarker is a gene for NEK2, the treatment compound is Compound 5, or a stereoisomer or mixture of stereoisomers, pharmaceutically acceptable salt, tautomer, prodrug, solvate, hydrate, co-crystal, clathrate, or polymorph thereof, and the second agent is an NEK2 inhibitor.

In one embodiment, the biomarker is a gene for NEK2, the treatment compound is Compound 6, or a stereoisomer or mixture of stereoisomers, pharmaceutically acceptable salt, tautomer, prodrug, solvate, hydrate, co-crystal, clathrate, or polymorph thereof, and the second agent is an NEK2 inhibitor.

In one embodiment, provided herein is a method of treating cancer, which comprises administering to a patient a therapeutically effective amount of Compound 1, or a stereoisomer or mixture of stereoisomers, pharmaceutically acceptable salt, tautomer, prodrug, solvate, hydrate, co-crystal, clathrate, or polymorph thereof, in combination with a PLK1 inhibitor. In one embodiment, provided herein is a method of treating multiple myeloma, which comprises administering to a patient a therapeutically effective amount of Compound 1 in combination with BI2536.

In one embodiment, provided herein is a method of treating cancer, which comprises administering to a patient a therapeutically effective amount of Compound 1, or a stereoisomer or mixture of stereoisomers, pharmaceutically acceptable salt, tautomer, prodrug, solvate, hydrate, co-crystal, clathrate, or polymorph thereof, in combination with a BRD4 inhibitor. In one embodiment, provided herein is a method of treating multiple myeloma, which comprises administering to a patient a therapeutically effective amount of Compound 1 in combination with JQ1.

In one embodiment, provided herein is a method of treating cancer, which comprises administering to a patient a therapeutically effective amount of Compound 1, or a stereoisomer or mixture of stereoisomers, pharmaceutically acceptable salt, tautomer, prodrug, solvate, hydrate, co-crystal, clathrate, or polymorph thereof, in combination with a BET inhibitor. In one embodiment, provided herein is a method of treating multiple myeloma, which comprises administering to a patient a therapeutically effective amount of Compound 1 in combination with Compound A.

In one embodiment, provided herein is a method of treating cancer, which comprises administering to a patient a therapeutically effective amount of Compound 1, or a stereoisomer or mixture of stereoisomers, pharmaceutically acceptable salt, tautomer, prodrug, solvate, hydrate, co-crystal, clathrate, or polymorph thereof, in combination with an NEK2 inhibitor. In one embodiment, provided herein is a method of treating multiple myeloma, which comprises administering to a patient a therapeutically effective amount of Compound 1 in combination with JH295. In one embodiment, provided herein is a method of treating multiple myeloma, which comprises administering to a patient a therapeutically effective amount of Compound 1 in combination with rac-CCT 250863.

In one embodiment, provided herein is a method of treating cancer, which comprises administering to a patient a therapeutically effective amount of Compound 2, or a stereoisomer or mixture of stereoisomers, pharmaceutically acceptable salt, tautomer, prodrug, solvate, hydrate, co-crystal, clathrate, or polymorph thereof, in combination with a PLK1 inhibitor. In one embodiment, provided herein is a method of treating multiple myeloma, which comprises administering to a patient a therapeutically effective amount of Compound 2 in combination with BI2536.

In one embodiment, provided herein is a method of treating cancer, which comprises administering to a patient a therapeutically effective amount of Compound 2, or a stereoisomer or mixture of stereoisomers, pharmaceutically acceptable salt, tautomer, prodrug, solvate, hydrate, co-crystal, clathrate, or polymorph thereof, in combination with a BRD4 inhibitor. In one embodiment, provided herein is a method of treating multiple myeloma, which comprises administering to a patient a therapeutically effective amount of Compound 2 in combination with JQ1.

In one embodiment, provided herein is a method of treating cancer, which comprises administering to a patient a therapeutically effective amount of Compound 2, or a stereoisomer or mixture of stereoisomers, pharmaceutically acceptable salt, tautomer, prodrug, solvate, hydrate, co-crystal, clathrate, or polymorph thereof, in combination with a BET inhibitor. In one embodiment, provided herein is a method of treating multiple myeloma, which comprises administering to a patient a therapeutically effective amount of Compound 2 in combination with Compound A.

In one embodiment, provided herein is a method of treating cancer, which comprises administering to a patient a therapeutically effective amount of Compound 2, or a stereoisomer or mixture of stereoisomers, pharmaceutically acceptable salt, tautomer, prodrug, solvate, hydrate, co-crystal, clathrate, or polymorph thereof, in combination with an NEK2 inhibitor. In one embodiment, provided herein is a method of treating multiple myeloma, which comprises administering to a patient a therapeutically effective amount of Compound 2 in combination with JH295. In one embodiment, provided herein is a method of treating multiple myeloma, which comprises administering to a patient a therapeutically effective amount of Compound 2 in combination with rac-CCT 250863.

In one embodiment, provided herein is a method of treating cancer, which comprises administering to a patient a therapeutically effective amount of Compound 3, or a stereoisomer or mixture of stereoisomers, pharmaceutically acceptable salt, tautomer, prodrug, solvate, hydrate, co-crystal, clathrate, or polymorph thereof, in combination with a PLK1 inhibitor. In one embodiment, provided herein is a method of treating multiple myeloma, which comprises administering to a patient a therapeutically effective amount of Compound 3 in combination with BI2536.

In one embodiment, provided herein is a method of treating cancer, which comprises administering to a patient a therapeutically effective amount of Compound 3, or a stereoisomer or mixture of stereoisomers, pharmaceutically acceptable salt, tautomer, prodrug, solvate, hydrate, co-crystal, clathrate, or polymorph thereof, in combination with a BRD4 inhibitor. In one embodiment, provided herein is a method of treating multiple myeloma, which comprises administering to a patient a therapeutically effective amount of Compound 3 in combination with JQ1.

In one embodiment, provided herein is a method of treating cancer, which comprises administering to a patient a therapeutically effective amount of Compound 3, or a stereoisomer or mixture of stereoisomers, pharmaceutically acceptable salt, tautomer, prodrug, solvate, hydrate, co-crystal, clathrate, or polymorph thereof, in combination with a BET inhibitor. In one embodiment, provided herein is a method of treating multiple myeloma, which comprises administering to a patient a therapeutically effective amount of Compound 3 in combination with Compound A.

In one embodiment, provided herein is a method of treating cancer, which comprises administering to a patient a therapeutically effective amount of Compound 3, or a stereoisomer or mixture of stereoisomers, pharmaceutically acceptable salt, tautomer, prodrug, solvate, hydrate, co-crystal, clathrate, or polymorph thereof, in combination with an NEK2 inhibitor. In one embodiment, provided herein is a method of treating multiple myeloma, which comprises administering to a patient a therapeutically effective amount of Compound 3 in combination with JH295. In one embodiment, provided herein is a method of treating multiple myeloma, which comprises administering to a patient a therapeutically effective amount of Compound 3 in combination with rac-CCT 250863.

In one embodiment, provided herein is a method of treating cancer, which comprises administering to a patient a therapeutically effective amount of Compound 4, or a stereoisomer or mixture of stereoisomers, pharmaceutically acceptable salt, tautomer, prodrug, solvate, hydrate, co-crystal, clathrate, or polymorph thereof, in combination with a PLK1 inhibitor. In one embodiment, provided herein is a method of treating multiple myeloma, which comprises administering to a patient a therapeutically effective amount of Compound 4 or pharmaceutically acceptable salt thereof (e.g., a hydrochloride salt of Compound 4) in combination with BI2536.

In one embodiment, provided herein is a method of treating cancer, which comprises administering to a patient a therapeutically effective amount of Compound 4, or a stereoisomer or mixture of stereoisomers, pharmaceutically acceptable salt, tautomer, prodrug, solvate, hydrate, co-crystal, clathrate, or polymorph thereof, in combination with a BRD4 inhibitor. In one embodiment, provided herein is a method of treating multiple myeloma, which comprises administering to a patient a therapeutically effective amount of Compound 4 or pharmaceutically acceptable salt thereof (e.g., a hydrochloride salt of Compound 4) in combination with JQ1.

In one embodiment, provided herein is a method of treating cancer, which comprises administering to a patient a therapeutically effective amount of Compound 4, or a stereoisomer or mixture of stereoisomers, pharmaceutically acceptable salt, tautomer, prodrug, solvate, hydrate, co-crystal, clathrate, or polymorph thereof, in combination with a BET inhibitor. In one embodiment, provided herein is a method of treating multiple myeloma, which comprises administering to a patient a therapeutically effective amount of Compound 4 or pharmaceutically acceptable salt thereof (e.g., a hydrochloride salt of Compound 4) in combination with Compound A.

In one embodiment, provided herein is a method of treating cancer, which comprises administering to a patient a therapeutically effective amount of Compound 4, or a stereoisomer or mixture of stereoisomers, pharmaceutically acceptable salt, tautomer, prodrug, solvate, hydrate, co-crystal, clathrate, or polymorph thereof, in combination with an NEK2 inhibitor. In one embodiment, provided herein is a method of treating multiple myeloma, which comprises administering to a patient a therapeutically effective amount of Compound 4 or pharmaceutically acceptable salt thereof (e.g., a hydrochloride salt of Compound 4) in combination with JH295. In one embodiment, provided herein is a method of treating multiple myeloma, which comprises administering to a patient a therapeutically effective amount of Compound 4 or pharmaceutically acceptable salt thereof (e.g., a hydrochloride salt of Compound 4) in combination with rac-CCT 250863.

In one embodiment, provided herein is a method of treating cancer, which comprises administering to a patient a therapeutically effective amount of Compound 5, or a stereoisomer or mixture of stereoisomers, pharmaceutically acceptable salt, tautomer, prodrug, solvate, hydrate, co-crystal, clathrate, or polymorph thereof, in combination with a PLK1 inhibitor. In one embodiment, provided herein is a method of treating multiple myeloma, which comprises administering to a patient a therapeutically effective amount of Compound 5 or pharmaceutically acceptable salt thereof (e.g., a hydrochloride salt of Compound 5) in combination with BI2536.

In one embodiment, provided herein is a method of treating cancer, which comprises administering to a patient a therapeutically effective amount of Compound 5, or a stereoisomer or mixture of stereoisomers, pharmaceutically acceptable salt, tautomer, prodrug, solvate, hydrate, co-crystal, clathrate, or polymorph thereof, in combination with a BRD4 inhibitor. In one embodiment, provided herein is a method of treating multiple myeloma, which comprises administering to a patient a therapeutically effective amount of Compound 5 or pharmaceutically acceptable salt thereof (e.g., a hydrochloride salt of Compound 5) in combination with JQ1.

In one embodiment, provided herein is a method of treating cancer, which comprises administering to a patient a therapeutically effective amount of Compound 5, or a stereoisomer or mixture of stereoisomers, pharmaceutically acceptable salt, tautomer, prodrug, solvate, hydrate, co-crystal, clathrate, or polymorph thereof, in combination with a BET inhibitor. In one embodiment, provided herein is a method of treating multiple myeloma, which comprises administering to a patient a therapeutically effective amount of Compound 5 or pharmaceutically acceptable salt thereof (e.g., a hydrochloride salt of Compound 5) in combination with Compound A.

In one embodiment, provided herein is a method of treating cancer, which comprises administering to a patient a therapeutically effective amount of Compound 5, or a stereoisomer or mixture of stereoisomers, pharmaceutically acceptable salt, tautomer, prodrug, solvate, hydrate, co-crystal, clathrate, or polymorph thereof, in combination with an NEK2 inhibitor. In one embodiment, provided herein is a method of treating multiple myeloma, which comprises administering to a patient a therapeutically effective amount of Compound 5 or pharmaceutically acceptable salt thereof (e.g., a hydrochloride salt of Compound 5) in combination with JH295. In one embodiment, provided herein is a method of treating multiple myeloma, which comprises administering to a patient a therapeutically effective amount of Compound 5 or pharmaceutically acceptable salt thereof (e.g., a hydrochloride salt of Compound 5) in combination with rac-CCT 250863.

In one embodiment, provided herein is a method of treating cancer, which comprises administering to a patient a therapeutically effective amount of Compound 6, or a stereoisomer or mixture of stereoisomers, pharmaceutically acceptable salt, tautomer, prodrug, solvate, hydrate, co-crystal, clathrate, or polymorph thereof, in combination with a PLK1 inhibitor. In one embodiment, provided herein is a method of treating multiple myeloma, which comprises administering to a patient a therapeutically effective amount of Compound 6 or pharmaceutically acceptable salt thereof (e.g., a hydrobromide salt of Compound 6) in combination with BI2536.

In one embodiment, provided herein is a method of treating cancer, which comprises administering to a patient a therapeutically effective amount of Compound 6, or a stereoisomer or mixture of stereoisomers, pharmaceutically acceptable salt, tautomer, prodrug, solvate, hydrate, co-crystal, clathrate, or polymorph thereof, in combination with a BRD4 inhibitor. In one embodiment, provided herein is a method of treating multiple myeloma, which comprises administering to a patient a therapeutically effective amount of Compound 6 or pharmaceutically acceptable salt thereof (e.g., a hydrobromide salt of Compound 6) in combination with JQ1.

In one embodiment, provided herein is a method of treating cancer, which comprises administering to a patient a therapeutically effective amount of Compound 6, or a stereoisomer or mixture of stereoisomers, pharmaceutically acceptable salt, tautomer, prodrug, solvate, hydrate, co-crystal, clathrate, or polymorph thereof, in combination with a BET inhibitor. In one embodiment, provided herein is a method of treating multiple myeloma, which comprises administering to a patient a therapeutically effective amount of Compound 6 or pharmaceutically acceptable salt thereof (e.g., a hydrobromide salt of Compound 6) in combination with Compound A.

In one embodiment, provided herein is a method of treating cancer, which comprises administering to a patient a therapeutically effective amount of Compound 6, or a stereoisomer or mixture of stereoisomers, pharmaceutically acceptable salt, tautomer, prodrug, solvate, hydrate, co-crystal, clathrate, or polymorph thereof, in combination with an NEK2 inhibitor. In one embodiment, provided herein is a method of treating multiple myeloma, which comprises administering to a patient a therapeutically effective amount of Compound 6 or pharmaceutically acceptable salt thereof (e.g., a hydrobromide salt of Compound 6) in combination with JH295. In one embodiment, provided herein is a method of treating multiple myeloma, which comprises administering to a patient a therapeutically effective amount of Compound 6 or pharmaceutically acceptable salt thereof (e.g., a hydrobromide salt of Compound 6) in combination with rac-CCT 250863.

In one embodiment, provided herein is a method of treating cancer, which comprises administering to a patient a therapeutically effective amount of Compound 7, or a stereoisomer or mixture of stereoisomers, pharmaceutically acceptable salt, tautomer, prodrug, solvate, hydrate, co-crystal, clathrate, or polymorph thereof, in combination with a PLK1 inhibitor. In one embodiment, provided herein is a method of treating multiple myeloma, which comprises administering to a patient a therapeutically effective amount of Compound 7 in combination with BI2536.

In one embodiment, provided herein is a method of treating cancer, which comprises administering to a patient a therapeutically effective amount of Compound 7, or a stereoisomer or mixture of stereoisomers, pharmaceutically acceptable salt, tautomer, prodrug, solvate, hydrate, co-crystal, clathrate, or polymorph thereof, in combination with a BRD4 inhibitor. In one embodiment, provided herein is a method of treating multiple myeloma, which comprises administering to a patient a therapeutically effective amount of Compound 7 in combination with JQ1.

In one embodiment, provided herein is a method of treating cancer, which comprises administering to a patient a therapeutically effective amount of Compound 7, or a stereoisomer or mixture of stereoisomers, pharmaceutically acceptable salt, tautomer, prodrug, solvate, hydrate, co-crystal, clathrate, or polymorph thereof, in combination with a BET inhibitor. In one embodiment, provided herein is a method of treating multiple myeloma, which comprises administering to a patient a therapeutically effective amount of Compound 7 in combination with Compound A.

In one embodiment, provided herein is a method of treating cancer, which comprises administering to a patient a therapeutically effective amount of Compound 7, or a stereoisomer or mixture of stereoisomers, pharmaceutically acceptable salt, tautomer, prodrug, solvate, hydrate, co-crystal, clathrate, or polymorph thereof, in combination with an NEK2 inhibitor. In one embodiment, provided herein is a method of treating multiple myeloma, which comprises administering to a patient a therapeutically effective amount of Compound 7 in combination with JH295. In one embodiment, provided herein is a method of treating multiple myeloma, which comprises administering to a patient a therapeutically effective amount of Compound 7 in combination with rac-CCT 250863.

Also provided herein are methods of treating patients who have been previously treated for multiple myeloma but are non-responsive to standard therapies, as well as those who have not previously been treated. Further encompassed are methods of treating patients who have undergone surgery in an attempt to treat multiple myeloma, as well as those who have not. Also provided herein are methods of treating patients who have been previously undergone transplant therapy, as well as those who have not.

The methods provided herein include treatment of multiple myeloma that is relapsed, refractory or resistant. The methods provided herein include prevention of multiple myeloma that is relapsed, refractory or resistant. The methods provided herein include management of multiple myeloma that is relapsed, refractory or resistant. In some such embodiments, the myeloma is primary, secondary, tertiary, quadruply or quintuply relapsed multiple myeloma. In one embodiment, the methods provided herein reduce, maintain or eliminate minimal residual disease (MRD). In one embodiment, provided herein is a method of increasing rate and/or durability of MRD negativity in multiple myeloma patients, comprising administering a therapeutically effective amount of a compound provided herein in combination with a second active agent provided herein. In one embodiment, methods provided herein encompass treating, preventing or managing various types of multiple myeloma, such as monoclonal gammopathy of undetermined significance (MGUS), low risk, intermediate risk, and high risk multiple myeloma, newly diagnosed multiple myeloma (including low risk, intermediate risk, and high risk newly diagnosed multiple myeloma), transplant eligible and transplant ineligible multiple myeloma, smoldering (indolent) multiple myeloma (including low risk, intermediate risk, and high risk smouldering multiple myeloma), active multiple myeloma, solitary plasmacytoma, extramedullary plasmacytoma, plasma cell leukemia, central nervous system multiple myeloma, light chain myeloma, non-secretory myeloma, Immunoglobulin D myeloma, and Immunoglobulin E myeloma, by administering a therapeutically effective amount of a compound described herein. In another embodiment, methods provided herein encompass treating, preventing or managing multiple myeloma characterized by genetic abnormalities, such as Cyclin D translocations (for example, t(11;14)(q13;q32); t(6;14)(p21;32); t(12;14)(p13;q32); or t(6;20);); MMSET translocations (for example, t(4;14)(p16;q32)); MAF translocations (for example, t(14;16)(q32;q32); t(20;22); t(16; 22)(q11;q13); or t(14;20)(q32;q11)); or other chromosome factors (for example, deletion of 17p13, or chromosome 13; del(17/17p), nonhyperdiploidy, and gain(1q)), by administering a therapeutically effective amount of a compound described herein. In one embodiment, the multiple myeloma is characterized according to the multiple myeloma International Staging System (ISS). In one embodiment, the multiple myeloma is Stage I multiple myeloma as characterized by ISS (e.g., serum β2 microglobulin<3.5 mg/L and serum albumin≥3.5 g/dL). In one embodiment, the multiple myeloma is Stage III multiple myeloma as characterized by ISS (e.g., serum β2 microglobulin>5.4 mg/L). In one embodiment, the multiple myeloma is Stage II multiple myeloma as characterized by ISS (e.g., not Stage I or III).

In some embodiments, the methods comprise administering a therapeutically effective amount of a compound provided herein in combination with a second active agent provided herein as induction therapy. In some embodiments, the methods comprise administering a therapeutically effective amount of a compound provided herein in combination with a second active agent provided herein as consolidation therapy. In some embodiments, the methods comprise administering a therapeutically effective amount of a compound provided herein in combination with a second active agent provided herein as maintenance therapy.

In one particular embodiment of the methods described herein, the multiple myeloma is plasma cell leukemia.

In one embodiment of the methods described herein, the multiple myeloma is high risk multiple myeloma. In some such embodiments, the high risk multiple myeloma is relapsed or refractory. In one embodiment, the high risk multiple myeloma is multiple myeloma that is relapsed within 12 months of first treatment. In yet another embodiment, the high risk multiple myeloma is multiple myeloma that is characterized by genetic abnormalities, for example, one or more of del(17/17p) and t(14;16)(q32;q32). In some such embodiments, the high risk multiple myeloma is relapsed or refractory to one, two or three previous treatments.

In one embodiment, the multiple myeloma is characterized by a p53 mutation. In one embodiment, the p53 mutation is a Q331 mutation. In one embodiment, the p53 mutation is an R273H mutation. In one embodiment, the p53 mutation is a K132 mutation. In one embodiment, the p53 mutation is a K132N mutation. In one embodiment, the p53 mutation is an R337 mutation. In one embodiment, the p53 mutation is an R337L mutation. In one embodiment, the p53 mutation is a W146 mutation. In one embodiment, the p53 mutation is an S261 mutation. In one embodiment, the p53 mutation is an S261T mutation. In one embodiment, the p53 mutation is an E286 mutation. In one embodiment, the p53 mutation is an E286K mutation. In one embodiment, the p53 mutation is an R175 mutation. In one embodiment, the p53 mutation is an R175H mutation. In one embodiment, the p53 mutation is an E258 mutation. In one embodiment, the p53 mutation is an E258K mutation. In one embodiment, the p53 mutation is an A161 mutation. In one embodiment, the p53 mutation is an A161T mutation.

In one embodiment, the multiple myeloma is characterized by homozygous deletion of p53. In one embodiment, the multiple myeloma is characterized by homozygous deletion of wild type p53.

In one embodiment, the multiple myeloma is characterized by wild type p53.

In one embodiment, the multiple myeloma is characterized by activation of one or more oncogenic drivers. In one embodiment, the one or more oncogenic drivers are selected from the group consisting of C-MAF, MAFB, FGFR3, MMset, Cyclin D1, and Cyclin D. In one embodiment, the multiple myeloma is characterized by activation of C-MAF. In one embodiment, the multiple myeloma is characterized by activation of MAFB. In one embodiment, the multiple myeloma is characterized by activation of FGFR3 and MMset. In one embodiment, the multiple myeloma is characterized by activation of C-MAF, FGFR3, and MMset. In one embodiment, the multiple myeloma is characterized by activation of Cyclin D1. In one embodiment, the multiple myeloma is characterized by activation of MAFB and Cyclin D1. In one embodiment, the multiple myeloma is characterized by activation of Cyclin D.

In one embodiment, the multiple myeloma is characterized by one or more chromosomal translocations. In one embodiment, the chromosomal translocation is t(14;16). In one embodiment, the chromosomal translocation is t(14;20). In one embodiment, the chromosomal translocation is t(4;14). In one embodiment, the chromosomal translocations are t(4;14) and t(14;16). In one embodiment, the chromosomal translocation is t(11;14). In one embodiment, the chromosomal translocation is t(6;20). In one embodiment, the chromosomal translocation is t(20;22). In one embodiment, the chromosomal translocations are t(6;20) and t(20;22). In one embodiment, the chromosomal translocation is t(16;22). In one embodiment, the chromosomal translocations are t(14;16) and t(16;22). In one embodiment, the chromosomal translocations are t(14;20) and t(11;14).

In one embodiment, the multiple myeloma is characterized by a Q331 p53 mutation, by activation of C-MAF, and by a chromosomal translocation at t(14;16). In one embodiment, the multiple myeloma is characterized by homozygous deletion of p53, by activation of C-MAF, and by a chromosomal translocation at t(14;16). In one embodiment, the multiple myeloma is characterized by a K132N p53 mutation, by activation of MAFB, and by a chromosomal translocation at t(14;20). In one embodiment, the multiple myeloma is characterized by wild type p53, by activation of FGFR3 and MMset, and by a chromosomal translocation at t(4;14). In one embodiment, the multiple myeloma is characterized by wild type p53, by activation of C-MAF, and by a chromosomal translocation at t(14;16). In one embodiment, the multiple myeloma is characterized by homozygous deletion of p53, by activation of FGFR3, MMset, and C-MAF, and by chromosomal translocations at t(4;14) and t(14;16). In one embodiment, the multiple myeloma is characterized by homozygous deletion of p53, by activation of Cyclin D1, and by a chromosomal translocation at t(11;14). In one embodiment, the multiple myeloma is characterized by an R337L p53 mutation, by activation of Cyclin D1, and by a chromosomal translocation at t(11;14). In one embodiment, the multiple myeloma is characterized by a W146 p53 mutation, by activation of FGFR3 and MMset, and by a chromosomal translocation at t(4;14). In one embodiment, the multiple myeloma is characterized by an S261T p53 mutation, by activation of MAFB, and by chromosomal translocations at t(6;20) and t(20;22). In one embodiment, the multiple myeloma is characterized by an E286K p53 mutation, by activation of FGFR3 and MMset, and by a chromosomal translocation at t(4;14). In one embodiment, the multiple myeloma is characterized by an R175H p53 mutation, by activation of FGFR3 and MMset, and by a chromosomal translocation at t(4;14). In one embodiment, the multiple myeloma is characterized by an E258K p53 mutation, by activation of C-MAF, and by chromosomal translocations at t(14;16) and t(16;22). In one embodiment, the multiple myeloma is characterized by wild type p53, by activation of MAFB and Cyclin D1, and by chromosomal translocations at t(14;20) and t(11;14). In one embodiment, the multiple myeloma is characterized by an A161T p53 mutation, by activation of Cyclin D, and by a chromosomal translocation at t(11;14).

In some embodiments of the methods described herein, the multiple myeloma is transplant eligible newly diagnosed multiple myeloma. In another embodiment, the multiple myeloma is transplant ineligible newly diagnosed multiple myeloma.

In yet other embodiments, the multiple myeloma is characterized by early progression (for example less than 12 months) following initial treatment. In still other embodiments, the multiple myeloma is characterized by early progression (for example less than 12 months) following autologous stem cell transplant. In another embodiment, the multiple myeloma is refractory to lenalidomide. In another embodiment, the multiple myeloma is refractory to pomalidomide. In some such embodiments, the multiple myeloma is predicted to be refractory to pomalidomide (for example, by molecular characterization). In another embodiment, the multiple myeloma is relapsed or refractory to 3 or more treatments and was exposed to a proteasome inhibitor (for example, bortezomib, carfilzomib, ixazomib, oprozomib, or marizomib) and an immunomodulatory compound (for example thalidomide, lenalidomide, pomalidomide, iberdomide, or avadomide), or double refractory to a proteasome inhibitor and an immunomodulatory compound. In still other embodiments, the multiple myeloma is relapsed or refractory to 3 or more prior therapies, including for example, a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide) or double refractory to a proteasome inhibitor or immunomodulatory compound and a CD38 mAb. In still other embodiments, the multiple myeloma is triple refractory, for example, the multiple myeloma is refractory to a proteasome inhibitor (for example, bortezomib, carfilzomib, ixazomib, oprozomib or marizomib), an immunomodulatory compound (for example thalidomide, lenalidomide, pomalidomide, iberdomide, or avadomide), and one other active agent, as described herein.

In certain embodiments, provided herein are methods of treating, preventing, and/or managing multiple myeloma, including relapsed/refractory multiple myeloma in patients with impaired renal function or a symptom thereof, comprising administering a therapeutically effective amount of a compound provided herein in combination with a second active agent provided herein, to a patient having relapsed/refractory multiple myeloma with impaired renal function.

In certain embodiments, provided herein are methods of treating, preventing, and/or managing multiple myeloma, including relapsed or refractory multiple myeloma in frail patients or a symptom thereof, comprising administering a therapeutically effective amount of a compound provided herein in combination with a second active agent provided herein, to a frail patient having multiple myeloma. In some such embodiments, the frail patient is characterized by ineligibility for induction therapy, or intolerance to dexamethasone treatment. In some such embodiment the frail patient is elderly, for example, older than 65 years old.

In certain embodiments, provided herein are methods of treating, preventing or managing multiple myeloma, comprising administering to a patient a therapeutically effective amount of a compound provided herein in combination with a second active agent provided herein, wherein the multiple myeloma is fourth line relapsed/refractory multiple myeloma.

In certain embodiments, provided herein are methods of treating, preventing or managing multiple myeloma, comprising administering to a patient a therapeutically effective amount of a compound provided herein in combination with a second active agent provided herein, as induction therapy, wherein the multiple myeloma is newly diagnosed, transplant-eligible multiple myeloma.

In certain embodiments, provided herein are methods of treating, preventing or managing multiple myeloma, comprising administering to a patient a therapeutically effective amount of a compound provided herein in combination with a second active agent provided herein, as maintenance therapy after other therapy or transplant, wherein the multiple myeloma is newly diagnosed, transplant-eligible multiple myeloma prior to the other therapy or transplant.

In certain embodiments, provided herein are methods of treating, preventing or managing multiple myeloma, comprising administering to a patient a therapeutically effective amount of a compound provided herein in combination with a second active agent provided herein, as maintenance therapy after other therapy or transplant. In some embodiments, the multiple myeloma is newly diagnosed, transplant-eligible multiple myeloma prior to the other therapy and/or transplant. In some embodiments, the other therapy prior to transplant is treatment with chemotherapy or a compound provided herein.

In certain embodiments, provided herein are methods of treating, preventing or managing multiple myeloma, comprising administering to a patient a therapeutically effective amount of a compound provided herein in combination with a second active agent provided herein, wherein the multiple myeloma is high risk multiple myeloma, that is relapsed or refractory to one, two or three previous treatments.

In certain embodiments, provided herein are methods of treating, preventing or managing multiple myeloma, comprising administering to a patient a therapeutically effective amount of a compound provided herein in combination with a second active agent provided herein, wherein the multiple myeloma is newly diagnosed, transplant-ineligible multiple myeloma.

In certain embodiments, the patient to be treated with one of the methods provided herein has not been treated with multiple myeloma therapy prior to the administration of a compound provided herein in combination with a second active agent provided herein. In certain embodiments, the patient to be treated with one of the methods provided herein has been treated with multiple myeloma therapy prior to the administration of a compound provided herein in combination with a second active agent provided herein. In certain embodiments, the patient to be treated with one of the methods provided herein has developed drug resistance to the anti-multiple myeloma therapy. In some such embodiments, the patient has developed resistance to one, two, or three anti-multiple myeloma therapies, wherein the therapies are selected from a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide).

The methods provided herein encompass treating a patient regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old. In one embodiment, the subject is an elderly multiple myeloma subject, such as a subject older than 65 years old. In one embodiment, the subject is an elderly multiple myeloma subject, such as a subject older than 75 years old.

### E. Dosing of Second Active Agents

In one embodiment, the specific amount (dosage) of a second active agent provided herein as used in the methods provided herein is determined by factors such as the specific agent used, the type of multiple myeloma being treated or managed, the severity and stage of disease, the amount of a compound provided herein, and any optional additional active agents concurrently administered to the patient.

In one embodiment, the dosage of a second active agent provided herein as used in the methods provided herein is determined based on a commercial package insert of medicament (e.g., a label) as approved by the FDA or a similar regulatory agency of a country other than the USA for said active agent. In one embodiment, the dosage of a second active agent provided herein as used in the methods provided herein is a dosage approved by the FDA or a similar regulatory agency of a country other than the USA for said active agent. In one embodiment, the dosage of a second active agent provided herein as used in the methods provided herein is a dosage used in a human clinical trial for said active agent. In one embodiment, the dosage of a second active agent provided herein as used in the methods provided herein is lower than a dosage approved by the FDA or a similar regulatory agency of a country other than the USA for said active agent or a dosage used in a human clinical trial for said active agent, depending on, e.g., the synergistic effects between the second active agent and a compound provided herein.

In one embodiment, the second active agent used in the methods provided herein is a BTK inhibitor. In one embodiment, the BTK inhibitor (e.g., ibrutinib) is administered at a dosage of in the range of from about 140 mg to about 700 mg, from about 280 mg to about 560 mg, or from about 420 mg to about 560 mg once daily. In one embodiment, the BTK inhibitor (e.g., ibrutinib) is administered at a dosage of no more than about 700 mg, no more than about 560 mg, no more than about 420 mg, no more than about 280 mg, or no more than about 140 mg once daily. In one embodiment, the BTK inhibitor (e.g., ibrutinib) is administered at a dosage of about 560 mg once daily. In one embodiment, the BTK inhibitor (e.g., ibrutinib) is administered at a dosage of about 420 mg once daily. In one embodiment, the BTK inhibitor (e.g., ibrutinib) is administered at a dosage of about 280 mg once daily. In one embodiment, the BTK inhibitor (e.g., ibrutinib) is administered at a dosage of about 140 mg once daily. In one embodiment, the BTK inhibitor (e.g., ibrutinib) is administered orally.

In one embodiment, the second active agent used in the methods provided herein is an mTOR inhibitor. In one embodiment, the mTOR inhibitor (e.g., everolimus) is administered at a dosage of in the range of from about 1 mg to about 20 mg, from about 2.5 mg to about 15 mg, or from about 5 mg to about 10 mg once daily. In one embodiment, the mTOR inhibitor (e.g., everolimus) is administered at a dosage of no more than about 20 mg, no more than about 15 mg, no more than about 10 mg, no more than about 5 mg, or no more than about 2.5 mg once daily. In one embodiment, the mTOR inhibitor (e.g., everolimus) is administered at a dosage of about 10 mg once daily. In one embodiment, the mTOR inhibitor (e.g., everolimus) is administered at a dosage of about 5 mg once daily. In one embodiment, the mTOR inhibitor (e.g., everolimus) is administered at a dosage of about 2.5 mg once daily. In one embodiment, the mTOR inhibitor (e.g., everolimus) is administered orally.

In one embodiment, the second active agent used in the methods provided herein is a PIM inhibitor. In one embodiment, the PIM inhibitor (e.g., LGH-447) is administered at a dosage of in the range of from about 30 mg to about 1000 mg, from about 70 mg to about 700 mg, from about 150 mg to about 500 mg, from about 200 mg to about 350 mg, or from about 250 mg to about 300 mg once daily. In one embodiment, the PIM inhibitor (e.g., LGH-447) is administered at a dosage of no more than about 700 mg, no more than about 500 mg, no more than about 350 mg, no more than about 300 mg, no more than about 250 mg, no more than about 200 mg, no more than about 150 mg, or no more than about 70 mg once daily. In one embodiment, the PIM inhibitor (e.g., LGH-447) is administered at a dosage of about 500 mg once daily. In one embodiment, the PIM inhibitor (e.g., LGH-447) is administered at a dosage of about 350 mg once daily. In one embodiment, the PIM inhibitor (e.g., LGH-447) is administered at a dosage of about 300 mg once daily. In one embodiment, the PIM inhibitor (e.g., LGH-447) is administered at a dosage of about 250 mg once daily. In one embodiment, the PIM inhibitor (e.g., LGH-447) is administered at a dosage of about 200 mg once daily. In one embodiment, the PIM inhibitor (e.g., LGH-447) is administered at a dosage of about 150 mg once daily. In one embodiment, the PIM inhibitor (e.g., LGH-447) is administered orally.

In one embodiment, the second active agent used in the methods provided herein is an IGF-1R inhibitor. In one embodiment, the IGF-1R inhibitor (e.g., linsitinib) is administered at a dosage of in the range of from about 100 mg to about 500 mg, from about 150 mg to about 450 mg, from about 200 mg to about 400 mg, or from about 250 mg to about 300 mg daily. In one embodiment, the IGF-1R inhibitor (e.g., linsitinib) is administered at a dosage of in the range of from about 50 mg to about 250 mg, from about 75 mg to about 225 mg, from about 100 mg to about 200 mg, or from about 125 mg to about 150 mg twice daily (BID). In one embodiment, the IGF-1R inhibitor (e.g., linsitinib) is administered at a dosage of no more than about 450 mg, no more than about 400 mg, no more than about 300 mg, no more than about 250 mg, no more than about 200 mg, or no more than about 150 mg daily. In one embodiment, the IGF-1R inhibitor (e.g., linsitinib) is administered at a dosage of no more than about 450 mg, no more than about 400 mg, no more than about 300 mg, no more than about 250 mg, no more than about 200 mg, or no more than about 150 mg daily. In one embodiment, the IGF-1R inhibitor (e.g., linsitinib) is administered at a dosage of no more than about 225 mg, no more than about 200 mg, no more than about 150 mg, no more than about 125 mg, no more than about 100 mg, or no more than about 75 mg twice daily. In one embodiment, the IGF-1R inhibitor (e.g., linsitinib) is administered at a dosage of about 450 mg, about 400 mg, about 300 mg, about 250 mg, about 200 mg, or about 150 mg daily. In one embodiment, the IGF-1R inhibitor (e.g., linsitinib) is administered at a dosage of about 225 mg, about 200 mg, about 150 mg, about 125 mg, about 100 mg, or about 75 mg twice daily. In one embodiment, the IGF-1R inhibitor (e.g., linsitinib) is administered on days 1 to 3 every 7 days. In one embodiment, the IGF-1R inhibitor (e.g., linsitinib) is administered orally.

In one embodiment, the second active agent used in the methods provided herein is an MEK inhibitor. In one embodiment, the MEK inhibitor (e.g., trametinib or trametinib dimethyl sulfoxide) is administered at a dosage of in the range of from about 0.25 mg to about 3 mg, from about 0.5 mg to about 2 mg, or from about 1 mg to about 1.5 mg once daily. In one embodiment, he MEK inhibitor (e.g., trametinib or trametinib dimethyl sulfoxide) is administered at a dosage of no more than about 2 mg, no more than about 1.5 mg, no more than about 1 mg, or no more than about 0.5 mg once daily. In one embodiment, he MEK inhibitor (e.g., trametinib or trametinib dimethyl sulfoxide) is administered at a dosage of about 2 mg once daily. In one embodiment, he MEK inhibitor (e.g., trametinib or trametinib dimethyl sulfoxide) is administered at a dosage of about 1.5 mg once daily. In one embodiment, he MEK inhibitor (e.g., trametinib or trametinib dimethyl sulfoxide) is administered at a dosage of about 1 mg once daily. In one embodiment, he MEK inhibitor (e.g., trametinib or trametinib dimethyl sulfoxide) is administered at a dosage of about 0.5 mg once daily. In one embodiment, he MEK inhibitor (e.g., trametinib or trametinib dimethyl sulfoxide) is administered orally.

In one embodiment, the second active agent used in the methods provided herein is an XPO1 inhibitor. In one embodiment, the XPO1 inhibitor (e.g., selinexor) is administered at a dosage of in the range of from about 30 mg to about 200 mg twice weekly, from about 45 mg to about 150 mg twice weekly, or from about 60 mg to about 100 mg twice weekly. In one embodiment, the XPO1 inhibitor (e.g., selinexor) is administered at a dosage of no more than about 100 mg, no more than about 80 mg, no more than about 60 mg, or no more than about 40 mg twice weekly. In one embodiment, the XPO1 inhibitor (e.g., selinexor) is administered at a dosage of about 20 mg, about 30 mg, about 40 mg, about 50 mg, about 60 mg, about 70 mg, about 80 mg, about 90 mg, or about 100 mg twice weekly. In one embodiment, the dosage is about 40 mg twice weekly. In one embodiment, the dosage is about 60 mg twice weekly. In one embodiment, the dosage is about 80 mg twice weekly. In one embodiment, the dosage is about 100 mg twice weekly. In one embodiment, the XPO1 inhibitor (e.g., selinexor) is administered orally.

In one embodiment, the second active agent used in the methods provided herein is a DOT1L inhibitor. In one embodiment, the DOT1L inhibitor (e.g., SGC0946) is administered at a dosage of in the range of from about 10 mg to about 500 mg, from about 25 mg to about 400 mg, from about 50 mg to about 300 mg, from about 75 mg to about 200 mg, or from about 100 mg to about 150 mg per day. In one embodiment, the DOT1L inhibitor (e.g., SGC0946) is administered at a dosage of no more than about 500 mg, no more than about 400 mg, no more than about 300 mg, no more than about 200 mg, no more than about 150 mg, no more than about 100 mg, no more than about 75 mg, no more than about 50 mg, or no more than about 25 mg per day. In one embodiment, the DOT1L inhibitor (e.g., SGC0946) is administered at a dosage of about 25 mg, about 50 mg, about 75 mg, about 100 mg, about 150 mg, about 200 mg, about 300 mg, about 400 mg, or about 500 mg. In one embodiment, the DOT1L inhibitor (e.g., SGC0946) is administered at a dosage of in the range of from about 18 mg/m² to about 126 mg/m², from about 36 mg/m² to about 108 mg/m², or from about 54 mg/m² to about 90 mg/m² per day. In one embodiment, the DOT1L inhibitor (e.g., SGC0946) is administered at a dosage of no more than about 126 mg/m², no more than about 108 mg/m², no more than about 90 mg/m², no more than about 72 mg/m², no more than about 54 mg/m², no more than about 36 mg/m², or no more than about 18 mg/m² per day. In one embodiment, the DOT1L inhibitor (e.g., SGC0946) is administered at a dosage of about 18 mg/m², about 36 mg/m², about 54 mg/m², about 72 mg/m², about 90 mg/m², about 108 mg/m², or about 126 mg/m² per day. In one embodiment, the DOT1L inhibitor (e.g., SGC0946) is administered orally. In one embodiment, the DOT1L inhibitor (e.g., SGC0946) is administered intravenously.

In one embodiment, the DOT1L inhibitor (e.g., pinometostat) is administered at a dosage of in the range of from about 18 mg/m² to about 108 mg/m², from about 36 mg/m² to about 90 mg/m², or from about 54 mg/m² to about 72 mg/m² per day. In one embodiment, the DOT1L inhibitor (e.g., pinometostat) is administered at a dosage of no more than about 108 mg/m², no more than about 90 mg/m², no more than about 72 mg/m², no more than about 54 mg/m², no more than about 36 mg/m², or no more than about 18 mg/m² per day. In one embodiment, the DOT1L inhibitor (e.g., pinometostat) is administered at a dosage of about 18 mg/m² per day. In one embodiment, the DOT1L inhibitor (e.g., pinometostat) is administered at a dosage of about 36 mg/m² per day. In one embodiment, the DOT1L inhibitor (e.g., pinometostat) is administered at a dosage of about 54 mg/m² per day. In one embodiment, the DOT1L inhibitor (e.g., pinometostat) is administered at a dosage of about 70 mg/m² per day. In one embodiment, the DOT1L inhibitor (e.g., pinometostat) is administered at a dosage of about 72 mg/m² per day. In one embodiment, the DOT1L inhibitor (e.g., pinometostat) is administered at a dosage of about 90 mg/m² per day. In one embodiment, the DOT1L inhibitor (e.g., pinometostat) is administered at a dosage of about 108 mg/m² per day. In one embodiment, the DOT1L inhibitor (e.g., pinometostat) is administered intravenously.

In one embodiment, the second active agent used in the methods provided herein is an EZH2 inhibitor. In one embodiment, the EZH2 inhibitor (e.g., tazemetostat) is administered at a dosage of in the range of from about 50 mg to about 1600 mg, from about 100 mg to about 800 mg, or from about 200 mg to about 400 mg twice daily (BID). In one embodiment, the EZH2 inhibitor (e.g., tazemetostat) is administered at a dosage of no more than about 800 mg, no more than about 600 mg, no more than about 400 mg, no more than about 200 mg, or no more than about 100 mg twice daily. In one embodiment, the EZH2 inhibitor (e.g., tazemetostat) is administered at a dosage of about 800 mg twice daily. In one embodiment, the EZH2 inhibitor (e.g., tazemetostat) is administered at a dosage of about 600 mg twice daily. In one embodiment, the EZH2 inhibitor (e.g., tazemetostat) is administered at a dosage of about 400 mg twice daily. In one embodiment, the EZH2 inhibitor (e.g., tazemetostat) is administered at a dosage of about 200 mg twice daily. In one embodiment, the EZH2 inhibitor (e.g., tazemetostat) is administered orally.

In one embodiment, the EZH2 inhibitor (e.g., CPI-1205) is administered at a dosage of in the range of from about 100 mg to about 3200 mg, from about 200 mg to about 1600 mg, or from about 400 mg to about 800 mg twice daily. In one embodiment, the EZH2 inhibitor (e.g., CPI-1205) is administered at a dosage of no more than about 3200 mg, no more than about 1600 mg, no more than about 800 mg, no more than about 400 mg, no more than about 200 mg, or no more than about 100 mg twice daily. In one embodiment, the EZH2 inhibitor (e.g., CPI-1205) is administered at a dosage of about 3200 mg twice daily. In one embodiment, the EZH2 inhibitor (e.g., CPI-1205) is administered at a dosage of about 1600 mg twice daily. In one embodiment, the EZH2 inhibitor (e.g., CPI-1205) is administered at a dosage of about 800 mg twice daily. In one embodiment, the EZH2 inhibitor (e.g., CPI-1205) is administered at a dosage of about 400 mg twice daily. In one embodiment, the EZH2 inhibitor (e.g., CPI-1205) is administered at a dosage of about 200 mg twice daily. In one embodiment, the EZH2 inhibitor (e.g., CPI-1205) is administered at a dosage of about 100 mg twice daily. In one embodiment, the EZH2 inhibitor (e.g., CPI-1205) is administered for one or more 28-day cycles. In one embodiment, the EZH2 inhibitor (e.g., CPI-1205) is administered orally.

In one embodiment, the second active agent used in the methods provided herein is a JAK2 inhibitor. In one embodiment, the JAK2 inhibitor (e.g., fedratinib) is administered at a dosage of in the range of from about 120 mg to about 680 mg, from about 240 mg to about 500 mg, or from about 300 mg to about 400 mg once daily. In one embodiment, the JAK2 inhibitor (e.g., fedratinib) is administered at a dosage of no more than about 680 mg, no more than about 500 mg, no more than about 400 mg, no more than about 300 mg, or no more than about 240 mg once daily. In one embodiment, the JAK2 inhibitor (e.g., fedratinib) is administered at a dosage of about 500 mg once daily. In one embodiment, the JAK2 inhibitor (e.g., fedratinib) is administered at a dosage of about 400 mg once daily. In one embodiment, the JAK2 inhibitor (e.g., fedratinib) is administered at a dosage of about 300 mg once daily.

In one embodiment, the second active agent used in the methods provided herein is a PLK1 inhibitor. In one embodiment, the PLK1 inhibitor (e.g., BI2536) is administered at a dosage of in the range of from about 20 mg to about 200 mg, from about 40 mg to about 100 mg, or from about 50 mg to about 60 mg per day. In one embodiment, the PLK1 inhibitor (e.g., BI2536) is administered at a dosage of no more than about 200 mg, no more than about 100 mg, no more than about 60 mg, no more than about 50 mg, no more than about 40 mg, or no more than about 20 mg per day. In one embodiment, the PLK1 inhibitor (e.g., BI2536) is administered at a dosage of about 200 mg, about 100 mg, about 60 mg, about 50 mg, about 40 mg, or about 20 mg per day. In one embodiment, the PLK1 inhibitor (e.g., BI2536) is administered at a dosage of about 200 mg once every 21-day cycle. In one embodiment, the PLK1 inhibitor (e.g., BI2536) is administered at a dosage of about 100 mg per day on days 1 and 8 of 21-day cycle. In one embodiment, the PLK1 inhibitor (e.g., BI2536) is administered at a dosage of about 50 mg per day on days 1 to 3 of 21-day cycle. In one embodiment, the PLK1 inhibitor (e.g., BI2536) is administered at a dosage of about 60 mg per day on days 1 to 3 of 21-day cycle. In one embodiment, the PLK1 inhibitor (e.g., BI2536) is administered intravenously.

In one embodiment, the second active agent used in the methods provided herein is an AURKB inhibitor. In one embodiment, the AURKB inhibitor (e.g., AZD1152) is administered at a dosage of in the range of from about 50 mg to about 200 mg, from about 75 mg to about 150 mg, or from about 100 mg to about 110 mg per day. In one embodiment, the AURKB inhibitor (e.g., AZD1152) is administered at a dosage of no more than about 200 mg, no more than about 150 mg, no more than about 110 mg, no more than about 100 mg, no more than about 75 mg, or no more than about 50 mg per day. In one embodiment, the AURKB inhibitor (e.g., AZD1152) is administered at a dosage of about 200 mg, about 150 mg, about 110 mg, about 100 mg, about 75 mg, or about 50 mg per day. In one embodiment, the AURKB inhibitor (e.g., AZD1152) is administered at a dosage described herein on days 1, 2, 15, and 16 of a 28-day cycle. In one embodiment, the AURKB inhibitor (e.g., AZD1152) is administered intravenously. In one embodiment, the AURKB inhibitor (e.g., AZD1152) is administered at a dosage of about 150 mg as a 48-hour continuous infusion every 14 days out of a 28-day cycle. In one embodiment, the AURKB inhibitor (e.g., AZD1152) is administered at a dosage of about 220 mg as 2×2-hour infusions every 14 days out of a 28-day cycle (e.g., 110 mg/day on days 1, 2, 15, and 16). In one embodiment, the AURKB inhibitor (e.g., AZD1152) is administered at a dosage of about 200 mg as a 2-hour infusion every 7 days. In one embodiment, the AURKB inhibitor (e.g., AZD1152) is administered at a dosage of about 450 mg as a 2-hour infusion every 14 days.

In one embodiment, the second active agent used in the methods provided herein is a BIRC5 inhibitor. In one embodiment, the BIRC5 inhibitor (e.g., YM155) is administered at a dosage of in the range of from about 2 mg/m² to about 15 mg/m², or from about 4 mg/m² to about 10 mg/m² per day. In one embodiment, the BIRC5 inhibitor (e.g., YM155) is administered at a dosage of no more than about 15 mg/m², no more than about 10 mg/m², no more than about 4.8 mg/m², no more than about 4 mg/m², or no more than about 2 mg/m² per day. In one embodiment, the BIRC5 inhibitor (e.g., YM155) is administered at a dosage of about 15 mg/m² per day. In one embodiment, the BIRC5 inhibitor (e.g., YM155) is administered at a dosage of about 10 mg/m² per day. In one embodiment, the BIRC5 inhibitor (e.g., YM155) is administered at a dosage of about 4.8 mg/m² per day. In one embodiment, the BIRC5 inhibitor (e.g., YM155) is administered at a dosage of about 4 mg/m² per day. In one embodiment, the BIRC5 inhibitor (e.g., YM155) is administered at a dosage of about 2 mg/m² per day. In one embodiment, the BIRC5 inhibitor (e.g., YM155) is administered intravenously. In one embodiment, the BIRC5 inhibitor (e.g., YM155) is administered at a dosage of about 4.8 mg/m²/day by about 168 hours continuous IV infusion every 3 weeks. In one embodiment, the BIRC5 inhibitor (e.g., YM155) is administered at a dosage of about 5 mg/m²/day by about 168 hours continuous IV infusion every 3 weeks. In one embodiment, the BIRC5 inhibitor (e.g., YM155) is administered at a dosage of about 10 mg/m²/day by about 72 hours continuous IV infusion every 3 weeks.

In one embodiment, the second active agent used in the methods provided herein is an BET inhibitor. In one embodiment, the BET inhibitor (e.g., birabresib) is administered at a dosage of in the range of from about 10 mg to about 160 mg, from about 20 mg to about 120 mg, or from about 40 mg to about 80 mg once daily. In one embodiment, the BET inhibitor (e.g., birabresib) is administered at a dosage of no more than about 160 mg, no more than about 120 mg, no more than about 80 mg, no more than about 40 mg, no more than about 20 mg, or no more than about 10 mg once daily. In one embodiment, the BET inhibitor (e.g., birabresib) is administered at a dosage of about 160 mg once daily. In one embodiment, the BET inhibitor (e.g., birabresib) is administered at a dosage of about 120 mg once daily. In one embodiment, the BET inhibitor (e.g., birabresib) is administered at a dosage of about 80 mg once daily. In one embodiment, the BET inhibitor (e.g., birabresib) is administered at a dosage of about 40 mg once daily. In one embodiment, the BET inhibitor (e.g., birabresib) is administered at a dosage of about 20 mg once daily. In one embodiment, the BET inhibitor (e.g., birabresib) is administered at a dosage of about 10 mg once daily. In one embodiment, the BET inhibitor (e.g., birabresib) is administered at a dosage described herein on Days 1 to 7 of a 21-day cycle. In one embodiment, the BET inhibitor (e.g., birabresib) is administered at a dosage described herein on Days 1 to 14 of a 21-day cycle. In one embodiment, the BET inhibitor (e.g., birabresib) is administered at a dosage described herein on Days 1 to 21 of a 21-day cycle. In one embodiment, the BET inhibitor (e.g., birabresib) is administered at a dosage described herein on Days 1 to 5 of a 7-day cycle. In one embodiment, the BET inhibitor (e.g., birabresib) is administered orally.

In one embodiment, the second active agent used in the methods provided herein is a DNA methyltransferase inhibitor. In one embodiment, the DNA methyltransferase inhibitor (e.g., azacitidine) is administered at a dosage of in the range of from about 25 mg/m² to about 150 mg/m², from about 50 mg/m² to about 125 mg/m², or from about 75 mg/m² to about 100 mg/m² daily. In one embodiment, the DNA methyltransferase inhibitor (e.g., azacitidine) is administered at a dosage of no more than about 150 mg/m², no more than about 125 mg/m², no more than about 100 mg/m², no more than about 75 mg/m², no more than about 50 mg/m², or no more than about 25 mg/m² daily. In one embodiment, the DNA methyltransferase inhibitor (e.g., azacitidine) is administered at a dosage of about 150 mg/m² daily. In one embodiment, the DNA methyltransferase inhibitor (e.g., azacitidine) is administered at a dosage of about 125 mg/m² daily. In one embodiment, the DNA methyltransferase inhibitor (e.g., azacitidine) is administered at a dosage of about 100 mg/m² daily. In one embodiment, the DNA methyltransferase inhibitor (e.g., azacitidine) is administered at a dosage of about 75 mg/m² daily. In one embodiment, the DNA methyltransferase inhibitor (e.g., azacitidine) is administered at a dosage of about 50 mg/m² daily. In one embodiment, the DNA methyltransferase inhibitor (e.g., azacitidine) is administered at a dosage of about 25 mg/m² daily. In one embodiment, the DNA methyltransferase inhibitor (e.g., azacitidine) is administered subcutaneously. In one embodiment, the DNA methyltransferase inhibitor (e.g., azacitidine) is administered intravenously.

In one embodiment, the DNA methyltransferase inhibitor (e.g., azacitidine) is administered at a dosage of in the range of from about 100 mg to about 500 mg, or from about 200 mg to about 400 mg once daily. In one embodiment, the DNA methyltransferase inhibitor (e.g., azacitidine) is administered at a dosage of no more than about 500 mg, no more than about 400 mg, no more than about 300 mg, no more than about 200 mg, or no more than about 100 mg once daily. In one embodiment, the DNA methyltransferase inhibitor (e.g., azacitidine) is administered at a dosage of about 500 mg once daily. In one embodiment, the DNA methyltransferase inhibitor (e.g., azacitidine) is administered at a dosage of about 400 mg once daily. In one embodiment, the DNA methyltransferase inhibitor (e.g., azacitidine) is administered at a dosage of about 300 mg once daily. In one embodiment, the DNA methyltransferase inhibitor (e.g., azacitidine) is administered at a dosage of about 200 mg once daily. In one embodiment, the DNA methyltransferase inhibitor (e.g., azacitidine) is administered at a dosage of about 100 mg once daily. In one embodiment, the DNA methyltransferase inhibitor (e.g., azacitidine) is administered at a dosage of in the range of from about 100 mg to about 300 mg, or from about 150 mg to about 250 mg twice daily. In one embodiment, the DNA methyltransferase inhibitor (e.g., azacitidine) is administered at a dosage of no more than about 300 mg, no more than about 250 mg, no more than about 200 mg, no more than about 150 mg, or no more than about 100 mg twice daily. In one embodiment, the DNA methyltransferase inhibitor (e.g., azacitidine) is administered at a dosage of about 300 mg twice daily. In one embodiment, the DNA methyltransferase inhibitor (e.g., azacitidine) is administered at a dosage of about 250 mg twice daily. In one embodiment, the DNA methyltransferase inhibitor (e.g., azacitidine) is administered at a dosage of about 200 mg twice daily. In one embodiment, the DNA methyltransferase inhibitor (e.g., azacitidine) is administered at a dosage of about 150 mg twice daily. In one embodiment, the DNA methyltransferase inhibitor (e.g., azacitidine) is administered at a dosage of about 100 mg twice daily. In one embodiment, the DNA methyltransferase inhibitor (e.g., azacitidine) is administered at a dosage described herein on Days 1 to 14 of a 28-day cycle. In one embodiment, the DNA methyltransferase inhibitor (e.g., azacitidine) is administered at a dosage described herein on Days 1 to 21 of a 28-day cycle. In one embodiment, the DNA methyltransferase inhibitor (e.g., azacitidine) is administered orally.

### F. Combination Therapy with Additional Active Agent

In one embodiment, the methods provided herein (combined use of a compound provided herein and a second active agent provided herein) additionally comprises administering to the patient an additional active agent (a third agent). In one embodiment, the third agent is a steroid.

The combined use of a compound provided herein and a second active agent provided herein can also be further combined or used in conjunction with (e.g. before, during, or after) conventional therapy including, but not limited to, surgery, biological therapy (including immunotherapy, for example with checkpoint inhibitors), radiation therapy, chemotherapy, stem cell transplantation, cell therapy, or other non-drug based therapy presently used to treat, prevent or manage cancer (e.g., multiple myeloma). The combined use of the compound provided herein, the second active agent provided herein, and conventional therapy may provide a unique treatment regimen that is unexpectedly effective in certain patients. Without being limited by theory, it is believed that a compound provided herein and a second active agent provided herein may provide additive or synergistic effects when given concurrently with conventional therapy.

As discussed elsewhere herein, encompassed herein is a method of reducing, treating and/or preventing adverse or undesired effects associated with conventional therapy including, but not limited to, surgery, chemotherapy, radiation therapy, biological therapy and immunotherapy. A compound provided herein a second active agent provided herein, and an additional active ingredient can be administered to a patient prior to, during, or after the occurrence of the adverse effect associated with conventional therapy. In one such embodiment, the additional active agent is dexamethasone.

The combined use of a compound provided herein and a second active agent provided herein can also be further combined or used in combination with other therapeutic agents useful in the treatment and/or prevention of multiple myeloma described herein. In one such embodiment, the additional active agent is dexamethasone.

In one embodiment, provided herein is a method of treating, preventing, or managing multiple myeloma, comprising administering to a patient a compound provided herein in combination with a second active agent provided herein, further in combination with one or more additional active agents, and optionally further in combination with radiation therapy, blood transfusions, or surgery.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second active agent provided herein) to the subject. The first therapy and the second therapy independently can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a third therapy (e.g., an additional prophylactic or therapeutic agent) to the subject. Quadruple therapy is also contemplated herein, as is quintuple therapy. In one embodiment, the third therapy is dexamethasone.

Administration of a compound provided herein, a second active agent provided herein, and one or more additional active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream).

The route of administration of a compound provided herein is independent of the route of administration of a second active agent provided herein as well as an additional therapy. In one embodiment, a compound provided herein is administered orally. In another embodiment, a compound provided herein is administered intravenously. In one embodiment, a second active agent provided herein is administered orally. In one embodiment, a second active agent provided herein is administered intravenously. Thus, in accordance with these embodiments, a compound provided herein is administered orally or intravenously, a second active agent provided herein is administered orally or intravenously, and the additional therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, a compound provided herein, a second active agent provided herein, and an additional therapy are administered by the same mode of administration, orally or by IV. In another embodiment, a compound provided herein is administered by one mode of administration, e.g., by IV, whereas a second active agent provided herein or the additional agent (an anti-multiple myeloma agent) is administered by another mode of administration, e.g., orally.

In one embodiment, the additional active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the additional active agent will depend on the specific agent used, the type of multiple myeloma being treated or managed, the severity and stage of disease, the amount of a compound provided herein, the amount of a second active agent provided herein, and any optional additional active agents concurrently administered to the patient.

One or more additional active ingredients or agents can be used together with a compound provided herein and a second active agent provided herein in the methods and compositions provided herein. Additional active agents can be large molecules (e.g., proteins), small molecules (e.g., synthetic inorganic, organometallic, or organic molecules), or cell therapies (e.g., CAR cells).

Examples of additional active agents that can be used in the methods and compositions described herein include one or more of melphalan, vincristine, cyclophosphamide, etoposide, doxorubicin, bendamustine, obinutuzmab, a proteasome inhibitor (for example, bortezomib, carfilzomib, ixazomib, oprozomib or marizomib), a histone deacetylase inhibitor (for example, panobinostat, ACY241), a BET inhibitor (for example, GSK525762A, OTX015, BMS-986158, TEN-010, CPI-0610, INCB54329, BAY1238097, FT-1101, ABBV-075, BI 894999, GS-5829, GSK1210151A (I-BET-151), CPI-203, RVX-208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1(2H)-one, EP11313 and EP11336), a BCL2 inhibitor (for example, venetoclax or navitoclax), an MCL-1 inhibitor (for example, AZD5991, AMG176, MIK665, S64315, or S63845), an LSD-1 inhibitor (for example, ORY-1001, ORY-2001, INCB-59872, IMG-7289, TAK-418, GSK-2879552, 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof), a corticosteroid (for example, prednisone), dexamethasone; an antibody (for example, a CS1 antibody, such as elotuzumab; a CD38 antibody, such as daratumumab or isatuximab; or a BCMA antibody or antibody-conjugate, such as GSK2857916 or BI 836909), a checkpoint inhibitor (as described herein), or CAR cells (as described herein).

In one embodiment, the additional active agent used together with a compound provided herein, and a second active agent provided herein in the methods and compositions described herein is dexamethasone.

In some embodiments, the dexamethasone is administered at a 4 mg dose on days 1 and 8 of a 21 day cycle. In some other embodiments, the dexamethasone is administered at a 4 mg dose on days 1, 4, 8 and 11 of a 21 day cycle. In some embodiments, the dexamethasone is administered at a 4 mg dose on days 1, 8, and 15 of a 28 day cycle. In some other embodiments, the dexamethasone is administered at a 4 mg dose on days 1, 4, 8, 11, 15 and 18 of a 28 day cycle. In some embodiments, the dexamethasone is administered at a 4 mg dose on days 1, 8, 15, and 22 of a 28 day cycle. In one such embodiment, the dexamethasone is administered at a 4 mg dose on days 1, 10, 15, and 22 of Cycle 1. In some embodiments, the dexamethasone is administered at a 4 mg dose on days 1, 3, 15, and 17 of a 28 day cycle. In one such embodiment, the dexamethasone is administered at a 4 mg dose on days 1, 3, 14, and 17 of Cycle 1.

In some other embodiments, the dexamethasone is administered at an 8 mg dose on days 1 and 8 of a 21 day cycle. In some other embodiments, the dexamethasone is administered at an 8 mg dose on days 1, 4, 8 and 11 of a 21 day cycle. In some embodiments, the dexamethasone is administered at an 8 mg dose on days 1, 8, and 15 of a 28 day cycle. In some other embodiments, the dexamethasone is administered at an 8 mg dose on days 1, 4, 8, 11, 15 and 18 of a 28 day cycle. In some embodiments, the dexamethasone is administered at an 8 mg dose on days 1, 8, 15, and 22 of a 28 day cycle. In one such embodiment, the dexamethasone is administered at an 8 mg dose on days 1, 10, 15, and 22 of Cycle 1. In some embodiments, the dexamethasone is administered at an 8 mg dose on days 1, 3, 15, and 17 of a 28 day cycle. In one such embodiment, the dexamethasone is administered at an 8 mg dose on days 1, 3, 14, and 17 of Cycle 1.

In some embodiments, the dexamethasone is administered at a 10 mg dose on days 1 and 8 of a 21 day cycle. In some other embodiments, the dexamethasone is administered at a 10 mg dose on days 1, 4, 8 and 11 of a 21 day cycle. In some embodiments, the dexamethasone is administered at a 10 mg dose on days 1, 8, and 15 of a 28 day cycle. In some other embodiments, the dexamethasone is administered at a 10 mg dose on days 1, 4, 8, 11, 15 and 18 of a 28 day cycle. In some embodiments, the dexamethasone is administered at a 10 mg dose on days 1, 8, 15, and 22 of a 28 day cycle. In one such embodiment, the dexamethasone is administered at a 10 mg dose on days 1, 10, 15, and 22 of Cycle 1. In some embodiments, the dexamethasone is administered at a 10 mg dose on days 1, 3, 15, and 17 of a 28 day cycle. In one such embodiment, the dexamethasone is administered at a 10 mg dose on days 1, 3, 14, and 17 of Cycle 1.

In some embodiments, the dexamethasone is administered at a 20 mg dose on days 1 and 8 of a 21 day cycle. In some other embodiments, the dexamethasone is administered at a 20 mg dose on days 1, 4, 8 and 11 of a 21 day cycle. In some embodiments, the dexamethasone is administered at a 20 mg dose on days 1, 8, and 15 of a 28 day cycle. In some other embodiments, the dexamethasone is administered at a 20 mg dose on days 1, 4, 8, 11, 15 and 18 of a 28 day cycle. In some embodiments, the dexamethasone is administered at a 20 mg dose on days 1, 8, 15, and 22 of a 28 day cycle. In one such embodiment, the dexamethasone is administered at a 20 mg dose on days 1, 10, 15, and 22 of Cycle 1. In some embodiments, the dexamethasone is administered at a 20 mg dose on days 1, 3, 15, and 17 of a 28 day cycle. In one such embodiment, the dexamethasone is administered at a 20 mg dose on days 1, 3, 14, and 17 of Cycle 1.

In some embodiments, the dexamethasone is administered at a 40 mg dose on days 1 and 8 of a 21 day cycle. In some other embodiments, the dexamethasone is administered at a 40 mg dose on days 1, 4, 8 and 11 of a 21 day cycle. In some embodiments, the dexamethasone is administered at a 40 mg dose on days 1, 8, and 15 of a 28 day cycle. In one such embodiment, the dexamethasone is administered at a 40 mg dose on days 1, 10, 15, and 22 of Cycle 1. In some other embodiments, the dexamethasone is administered at a 40 mg dose on days 1, 4, 8, 11, 15 and 18 of a 28 day cycle. In other such embodiments, the dexamethasone is administered at a 40 mg dose on days 1, 8, 15, and 22 of a 28 day cycle. In other such embodiments, the dexamethasone is administered at a 40 mg dose on days 1, 3, 15, and 17 of a 28 day cycle. In one such embodiment, the dexamethasone is administered at a 40 mg dose on days 1, 3, 14, and 17 of Cycle 1.

In another embodiment, the additional active agent used together with a compound provided herein, and a second active agent provided herein in the methods and compositions described herein is bortezomib. In yet another embodiment, the additional active agent used together with a compound provided herein, and a second active agent provided herein in the methods and compositions described herein is daratumumab. In some such embodiments, the methods additionally comprise administration of dexamethasone. In some embodiments, the methods comprise administration of a compound provided herein, and a second active agent provided herein with a proteasome inhibitor as described herein, a CD38 inhibitor as described herein and a corticosteroid as described herein.

In certain embodiments, a compound provided herein, and a second active agent provided herein are administered in combination with checkpoint inhibitors. In one embodiment, one checkpoint inhibitor is used in combination with a compound provided herein, and a second active agent provided herein in connection with the methods provided herein. In another embodiment, two checkpoint inhibitors are used in combination with a compound provided herein, and a second active agent provided herein in connection with the methods provided herein. In yet another embodiment, three or more checkpoint inhibitors are used in combination with a compound provided herein, and a second active agent provided herein in connection with the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with one or more second active agents as described herein where appropriate for treating diseases described herein and understood in the art.

In certain embodiments, a compound provided herein and a second active agent provided herein can be used in combination with one or more immune cells expressing one or more chimeric antigen receptors (CARs) on their surface (e.g., a modified immune cell). Generally, CARs comprise an extracellular domain from a first protein (e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a V_{L} linked to V_{H} by a flexible linker, wherein said V_{L} and V_{H} are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD 117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMIB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin αvβ3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAA0205, Mart2, Mum-1, 2, and 3, neo-PAP, myosin class I, OS-9, pm1-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, p15(58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, or TPS.

In various specific embodiments, the tumor-associated antigen or tumor-specific antigen is an AML-related tumor antigens, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In a specific embodiment, the intracellular domain is a CD3ζ intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3ζ signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 1 05(11):4247-4254 (2005).

In certain embodiments, a compound provided herein and a second active agent provided herein are administered to patients with various types or stages of multiple myeloma in combination with chimeric antigen receptor (CAR) T-cells. In certain embodiments the CAR T cell in the combination targets B cell maturation antigen (BCMA), and in more specific embodiments, the CAR T cell is bb2121 or bb21217. In some embodiments, the CAR T cell is JCARH125.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### 8. EXAMPLES

Certain embodiments of the invention are illustrated by the following non-limiting examples.

### Example 1: PLK1 Inhibition Decreases Cell Proliferation in Multiple Myeloma Cell Lines

Cell lines. All MM cell lines (ATCC, Manassas, Va., USA) were routinely tested for Mycoplasma and maintained in RPMI 1640 medium supplemented with L-glutamine, fetal bovine serum, penicillin, and streptomycin (all from Invitrogen, Carlsbad, Calif.). These cell lines were authenticated regularly.

Antibodies. Several antibodies were used for immunoblotting and flow cytometry in these experiments including Plk1 (Cat #4513), Aiolos (Cat #15103), Ikaros (Cat #14859), CDC25C (Cat #4688), pCDC25C (Cat #4901), Cleaved caspase 3 (Cat #9664), Survivin (Cat #2803), Bcl2 (Cat #2872), BRD4 (Cat #13440), c-Myc (Cat #5605), pERK (Cat #4376), ERK (Cat #4695), IRF7 (Cat #13014), FOXM1 (Cat #5436), Phospho-Histone H3 (Ser10) (D2C8) (Alexa Fluor® 594 Conjugate) (Cat #8481), all from Cell signaling technologies (Danvers, Mass., USA), E2F2 (Cat #Ab-138515, Abcam, Cambridge, Mass., USA), CKS1B (Cat #36-6800, Invitrogen, Waltham, Mass., USA), NUF2 (Cat #NBP2-43779, Novus Saint Charles, Mo., USA), TOP2A (Cat #PA5-46814, Invitrogen, Waltham, Mass., USA), ETV4 (Cat #10684-1, Invitrogen, Waltham, Mass., USA), IRF5 (Cat #10547-1-AP, Proteintech, Rosemont, Ill.).

Proliferation and viability assays: Cell growth curves were determined by monitoring the viability of cells with Trypan blue exclusion on a Vi-Cell-XR (Becton Dickinson, Franklin Lakes, N.J., USA). Proliferation assays were performed in triplicate at least three times (n=3) using (3H)-thymidine incorporation. All data were plotted and analyzed using GraphPad Prism 7 (GraphPad Software, La Jolla, Calif., USA) software, represented as the mean with an error determined as ±s.d.

Immunoblotting: Immunoblot analysis was performed using WES kits, (Protein Simple, San Jose, Calif., USA) at least two times each (n≥2), where the best representative is shown.

RNA Extraction, Reverse Transcription, and Real-Time PCR Analysis: Total RNA was extracted using a RNeasy plus kit (Qiagen, Germantown, Md., USA) and reverse-transcribed using an iScrip reverse transcription kit (Bio-Rad, Philadelphia, Pa., USA). Quantitative real-time PCR (qPCR) analyses were performed using Taqman PCR Master Mix and the ViiA 7 Real-Time PCR System (Applied Biosystems, Foster City, Calif., USA). Gene expressions were calculated following normalization to GAPDH levels using the comparative CT method (ΔΔCT method). The primer sequences for qPCR are following:

PLK1 RT F:
(SEQ ID NO: 1)
CACAGTGTCAATGCCTCCAA,
PLK1 RT R:
(SEQ ID NO: 2)
GACCCAGAAGATGGGGATG,
ACTB RT F:
(SEQ ID NO: 3)
CTCTTCCAGCCTTCCTTCCT,
ACTB RT R:
(SEQ ID NO: 4)
GGATGTCCACGTCACACTTC.

ChIP-PCR and ChIP-seq studies: ChIP-PCR and ChIP-sequence experiments in H929 and DF15 cell lines were performed using standard methods. The primer sequences for ChIP-PCR are following:

PLK1 transcriptional start sites (TSS) ChIP F:
(SEQ ID NO: 5)
GCGCAGGCTTTTGTAACG,
PLK1 TSS ChIP R:
(SEQ ID NO: 6)
CTCCTCCCCGAATTCAAAC.

Flow cytometry: Annexin-V Alexa Fluor 488-conjugated antibody (Thermo Scientific, Waltham, Mass., USA) and To-Pro-3 (Thermo Scientific, Waltham, Mass., USA) were used according to the manufacturer's protocol and processed using Flow Jo software for at least three independent experiments. For cell cycle analysis, cells were stained with Propidium Iodide (PI) staining kit (Abcam, Cambridge, Mass., USA) and analysis was performed on Flow Jo V10 software. Staining of mitotic marker pHH3-Ser¹⁰ was also performed by pHH3-Ser¹⁰ and PI dual staining.

ConfocalImaging: Cells were cultured in chambered slides and fixed permeabilized for microscopy. Cells were incubated with primary antibodies targeting PLK1, CDC25C in 1× intracellular staining buffer for 2 hours in cold room. Cells were then stained with Alexa Flour 488 and 594 conjugated secondary antibodies for 30 mins at RT, washed and counter-stained with DAPI. Confocal images were captured using Nikon A1R (Melville, N.Y., USA).

Single cell transcript analysis: Single cell sequencing was performed following manufacturer's instructions using 10× genomics (Pleasanton, Calif., USA) kits. Datasets were analyzed Cell ranger pipeline of 10× genomics using Seurat algorithm.

shRNA knockdown: Doxycycline (DOX)-inducible shRNA constructs targeting PLK1 were generated by Cellecta (Mountain View, Calif., USA) using pRSITEP-U6Tet-(sh)-EF1-TetRep-2A-Puro plasmid. Luciferase negative control were generated as previously described (PMID: 21189262). Briefly, 293T cells were co-transfected with lentiviral packaging plasmid mix (Cellecta, Cat #CPCP-K2A) and pRSITEP-shRNA constructs. Viral particle was collected 48 after transfection and then concentrated 10-fold by Amicon Ultra-15 centrifugal filters. For infections, cells were incubated overnight with concentrated viral supernatants in the presence of 8 μg/ml polybrene. Cells were then washed to remove polybrene. At 48 hours post-infection, cells were selected with puromycin (1 μg/ml) for more than 3 weeks before experiments. The shRNA target sequences were

PLK1 shRNA1:
(SEQ ID NO: 7)
GTTCTTTACTTCTGGCTATAT;
PLK1 shRNA2:
(SEQ ID NO: 8)
CTGCACCGAAACCGAGTTATT.

Results.

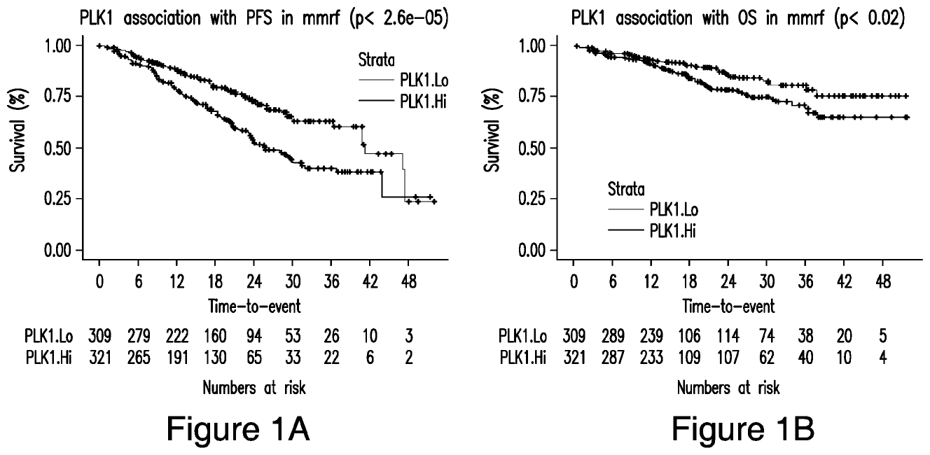

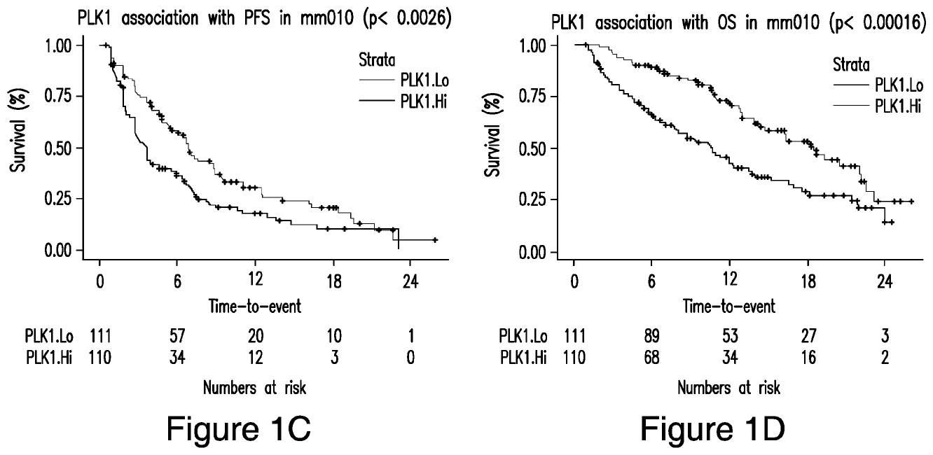

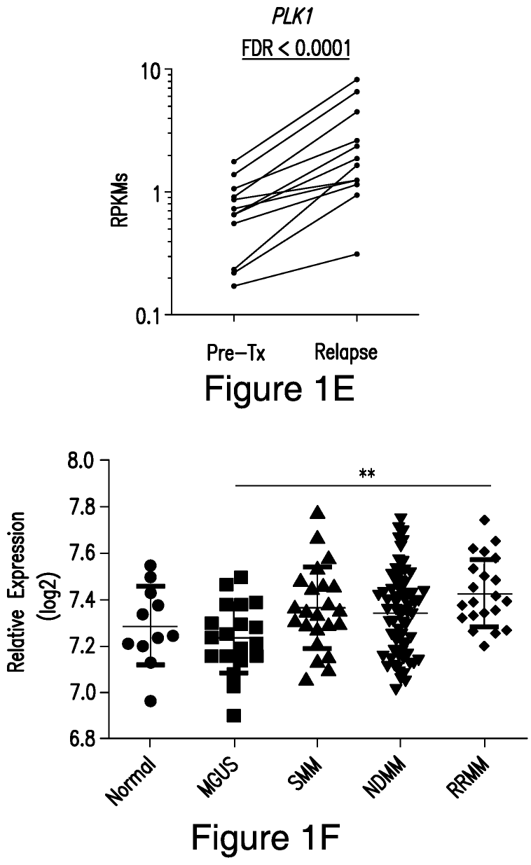

PLK1 upregulation is associated with high risk disease and relapse in MM patients. The expression of PLK1 was analyzed in newly diagnosed (MMRF) and relapse refractory (MM010) datasets. Changes in survival were depicted as progression free and overall survival. In both datasets, higher PLK1 expression was associated with significantly lower progression free and overall survival (FIGS. 1A-1D). The expression of PLK1 in various clusters of myeloma genome project (MGP) was further evaluated. PLK1 expression was found to be most upregulated in the high-risk cluster (data not shown). Analysis of PLK1 expression in 12 paired MM patient samples of sorted CD138+ cells obtained prior to lenalidomide treatment initiation and after development of resistance by RNA-seq. PLK1 expression was significantly (FDR<0.00001) upregulated in patients at relapse (FIG. 1E). Each of the twelve relapsed patients demonstrated an upregulation of PLK1 levels at the time of relapse. Analysis of the expression pattern of PLK1 across various stages of MM disease progression and relapse in Mayo clinic gene expression datasets, showed a significant increase in PLK1 expression in relapsed cohort of patients with a trend of increase upon disease progression (FIG. 1F).

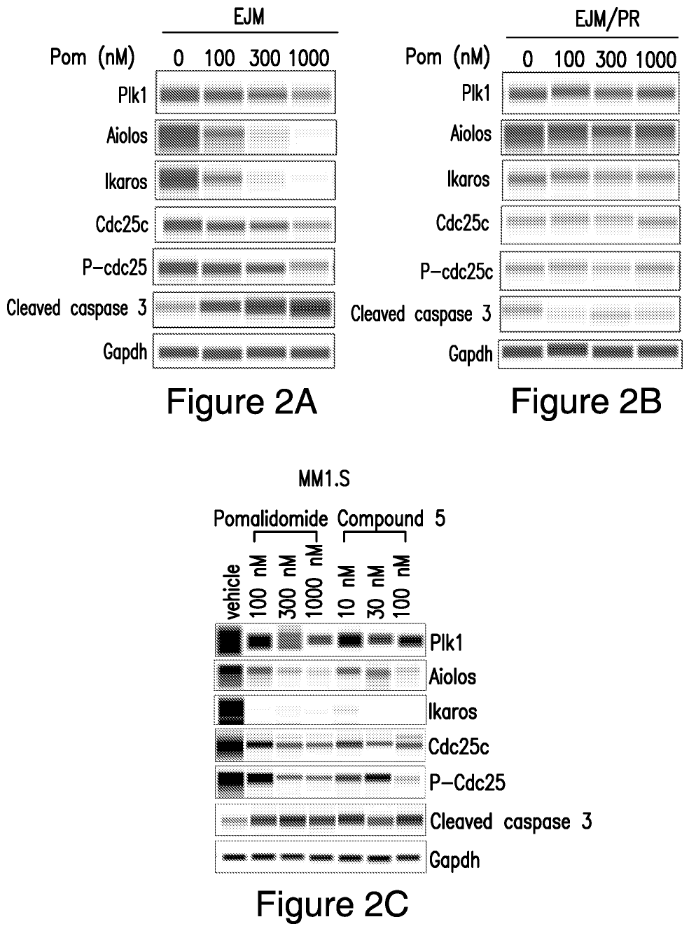

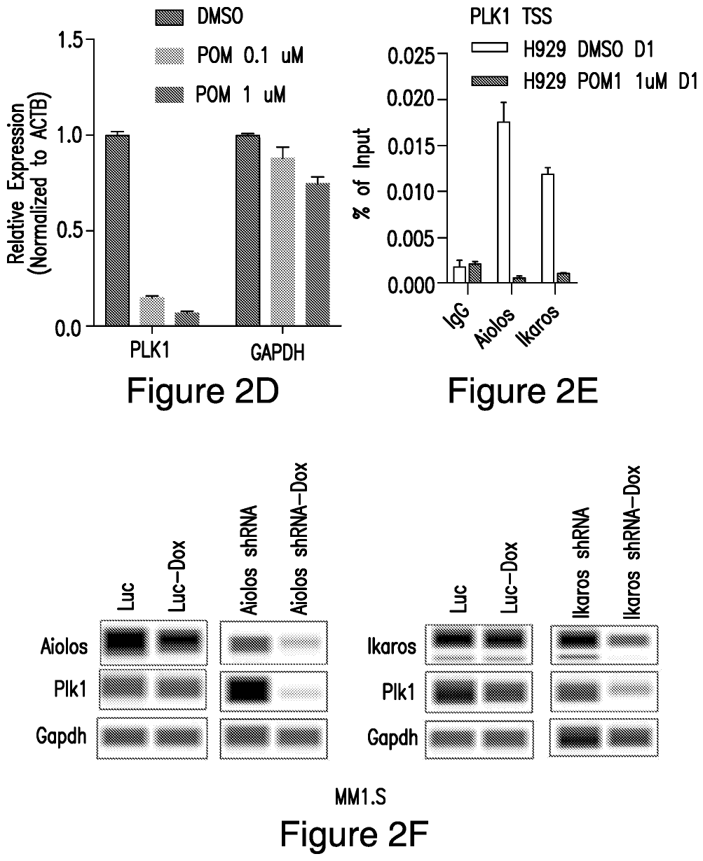

PLK1 signaling is downregulated in response to antiproliferative compounds in sensitive cells. The effects of pomalidomide in isogenic sensitive (EJM) and resistant (EJM-PR) and MM1.S cell lines was analyzed. Based on changes in proliferation, MM1.S cell line showed highest sensitivity to pomalidomide and EJM-PR was the most resistant. To determine the role of PLK1 in pomalidomide response, EJM and EJM-PR cell lines were treated with pomalidomide and analyzed changes in PLK1 levels and downstream signaling. Pomalidomide treatment caused a dose dependent decrease in PLK1 levels and its downstream effector pCDC25C and CDC25C, only in sensitive cells (FIG. 2A and FIG. 2B). CDC25C gene expression significantly correlates with PLK1 expression in MGP. In response to pomalidomide, cereblon substrates Ikaros and Aiolos were also downregulated in pomalidomide sensitive cells. Antiproliferative agents, such as Compound 5, have shown to be more effective in mediating substrate degradation. MMS.1 cells treated with increasing concentrations of pomalidomide and Compound 5, showed a dose dependent decrease in PLK1 signaling by both inhibitors (FIG. 2C). Consistent with the differences in activity of these two inhibitors, Compound 5 demonstrated a decrease in PLK1 levels and its downstream signaling at ten times lower dose compared to pomalidomide. MMS.1 cell line demonstrated a more prominent decrease in PLK1 levels at matched doses of pomalidomide compared to EJM cells, which correlate with the differences in sensitivity of the two cell lines to pomalidomide. The changes in PLK1 transcript levels were further examined in response to pomalidomide treatment in MM1. S cells and the treatments decreased PLK1 transcript levels in a dose dependent manner (FIG. 2D). Confocal microscopy was performed to study changes in PLK1 and CDC25C staining in MM1.S cells and a decrease in PLK1 levels and simultaneous decrease in CDC25C staining in response to pomalidomide and Compound 5 treatments was observed. Further, ChIP-PCR analysis revealed the binding of Aiolos and Ikaros to transcriptional start sites (TSS) of PLK1, which was abrogated in response to pomalidomide (FIG. 2E). Further analysis of the ChIP-seq datasets of Aiolos confirmed binding of Aiolos on TSS of PLK1 with overlapping transcriptional activation H3K27Ac signature extrapolated from publicly available ChIP-seq datasets in GM12878 cell line (Encode project). Since changes in PLK1 levels are due to decrease in PLK1 transcription in response to antiproliferative compounds, the effects of Aiolos and Ikaros knockdowns on PLK1 levels using MM1.S cells with inducible expression of Aiolos and Ikaros shRNAs were analyzed. Both Aiolos and Ikaros knock down lead to a decrease in PLK1 levels (FIG. 2F), indicating transcriptional regulation of PLK1 by substrates of Cereblon.

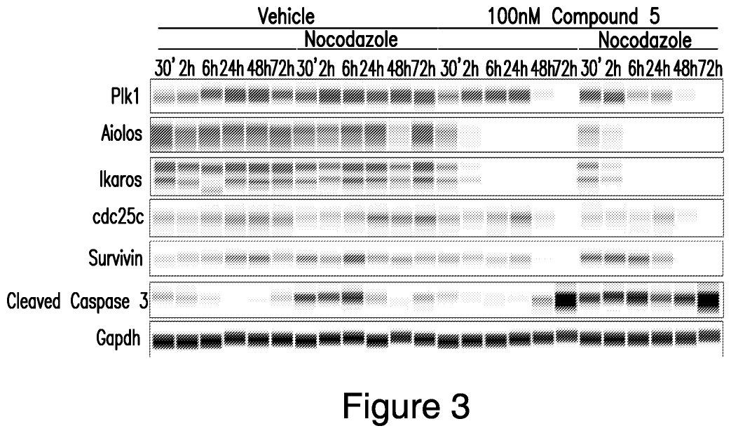

Compound 5 treatment caused a decrease in G2-M phase of cell cycle. Since, PLK1 plays an important role in G2 and mitotic phases of cell cycle, the changes in cell cycle in response to Compound 5 were examined and showed that Compound 5 treatments caused a dose dependent increase in sub-G1 (5.02, 4.98, 11.3, and 13.9 for vehicle, Compound 5 at 10 nM, Compound 5 at 30 nM, and Compound 5 at 100 nM, respectively) and G0-G1 populations (69.2, 75.8, 78.3, and 75.1 for vehicle, Compound 5 at 10 nM, Compound 5 at 30 nM, and Compound 5 at 100 nM, respectively) and a simultaneous decrease in G2-M population (16.3, 12.3, 6.94, and 6.27 for vehicle, Compound 5 at 10 nM, Compound 5 at 30 nM, and Compound 5 at 100 nM, respectively). The changes in phospho Ser10-histone H3 which is a specific marker for G2-M phase using flow cytometry were measured. Consistent with the overall cell cycle distribution, levels of phospho Ser10-histone H3 also decreased in a dose dependent manner in response to Compound 5 treatment (16.5, 9.3, 6.37, and 4.53 for vehicle, Compound 5 at 10 nM, Compound 5 at 30 nM, and Compound 5 at 100 nM, respectively). To prove that the observed changes in PLK1 signaling is not a consequence of a mitotic exit, the changes in PLK1 signaling after treating cells with Nocodazole and Compound 5 and their combination using various time points was analyzed. Nocodazole synchronizes cells in G2-M phase of cell cycle. At time points 30 minutes, 2 hours and 6 hours post rescue from overnight Nocodazole treatment, PLK1 levels were higher compared to vehicle condition (FIG. 3). Then, due to rescue from cell cycle synchronization post Nocodazole treatment, PLK1 levels normalized. In response to Compound 5 treatment, while Ikaros degradation began to happen after 30 minutes of treatments, downregulation of PLK1 and CDC25C levels was evident at 48 hours post treatment. In response to Nocodazole and Compound 5 combination treatment, the decrease in PLK1 levels was accelerated. Changes in cleaved caspase 3 inversely correlated with PLK1 levels, with an increase in cleaved caspase 3 at 48 and 72 hours post Compound 5 treatment with a decrease in PLK1 levels. Cell cycle studies matched with these time points. Nocodazole treatment showed an increase in G2-M cells at early time points of rescue. Compound 5 treatment caused an initial increase in G1 cells, followed an increase in sub-G1 and a decrease in G2-M cells at 48 and 72 hours (data not shown). In case of Nocodazole and Compound 5 combination treatment, an accelerated decrease in G2-M cells and a higher increase in sub-G1 cells was observed.

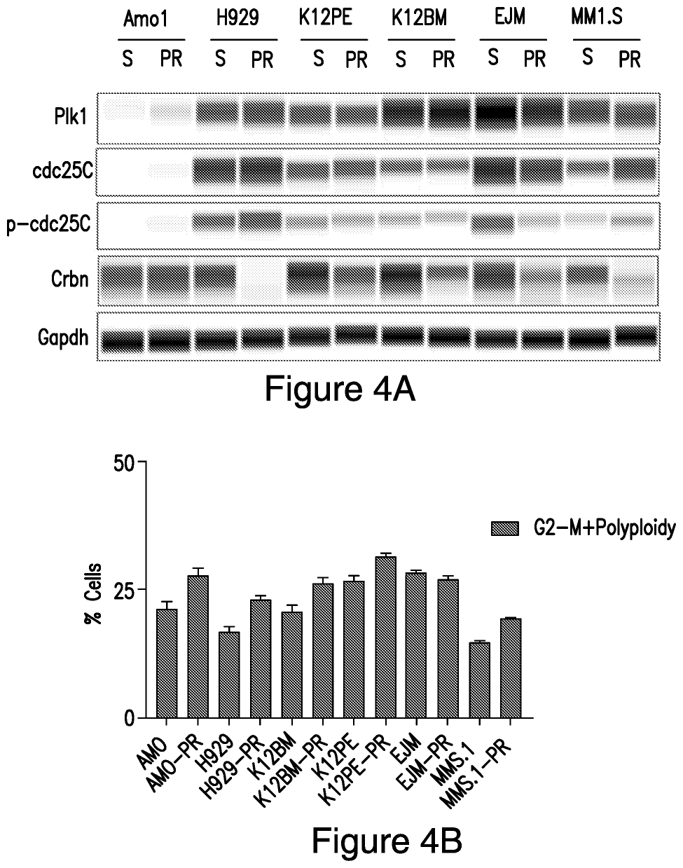

Pomalidomide resistant cells demonstrate activated PLK1 signaling and increased mitosis. To investigate the role of PLK1 in pomalidomide resistance, the levels of PLK1, CDC25C and pCDC25C and Cereblon in six pomalidomide sensitive and resistant isogenic pair of cell lines namely AMO1 and AMO1-PR (pomalidomide resistant), H929 and H929-PR, K12PE and K12PE-PR, K12BM and K12BM-PR, EJM and EJM-PR and MMS.1 and MMS.1PR was analyzed. These cell lines were developed by exposing them to increasing concentrations of pomalidomide over a period of three-four months. PLK1 levels were moderately upregulated in the resistant version of four of the six cell lines (FIG. 4A). The resistant cell lines also demonstrated variable loss in cereblon levels compared to parental cells. Asynchronous cell cycle distribution studies comparing the parental and resistant cell lines demonstrated an increased proportion of G2-M cells in five of six resistant cell lines (FIG. 4B). To further analyze the changes in PLK1 expression in various phases of cell cycle between sensitive and resistant cell lines, single cell RNA sequencing in AMO1 and AMO1-PR cell lines was performed. Gene expression clustering analysis based on cell cycle signature genes revealed a substantially restricted expression of PLK1 in G2-M phase of cell cycle and confirmed the upregulated expression of PLK1 in AMO1-PR cells compared to the AMO1-parental (data not shown). Aiolos and Ikaros were found to be expressed more ubiquitously across the different phases of cell cycle (data not shown).

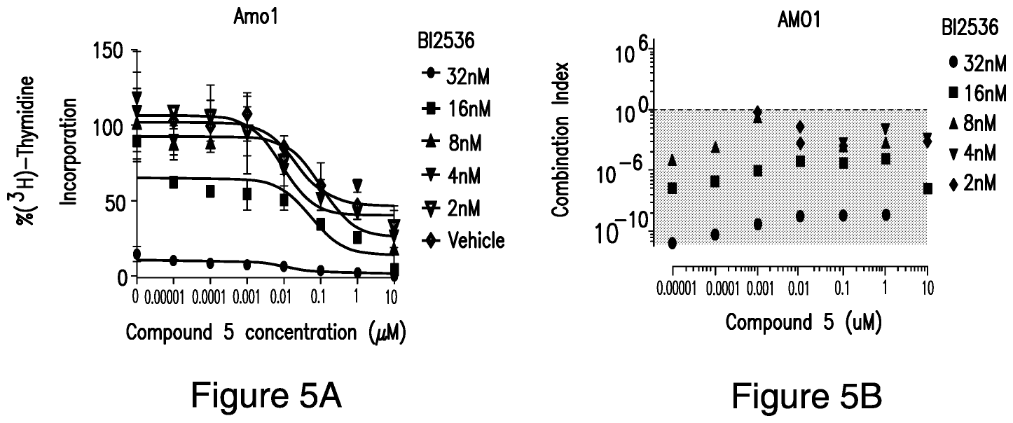

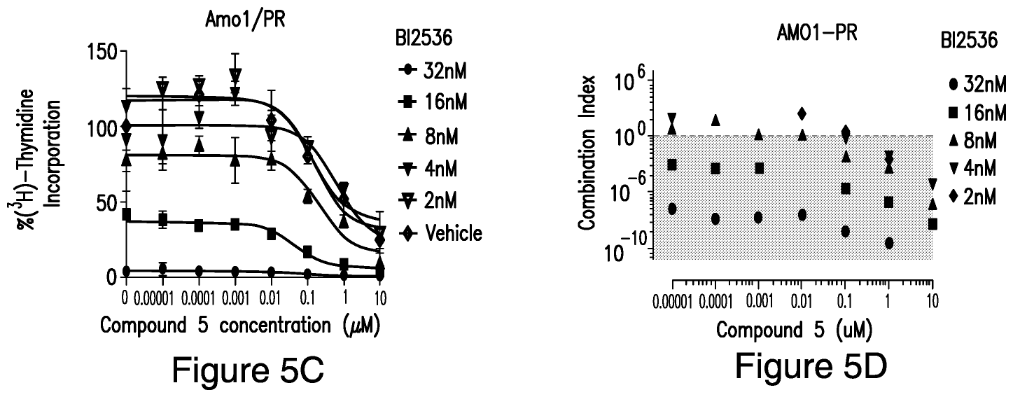

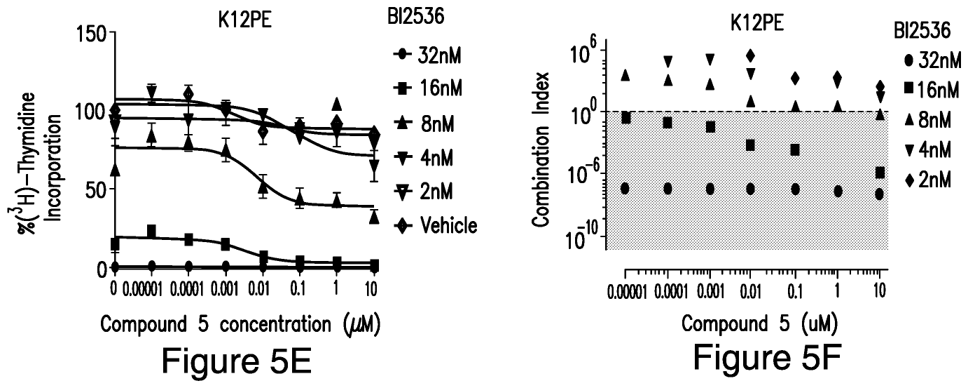

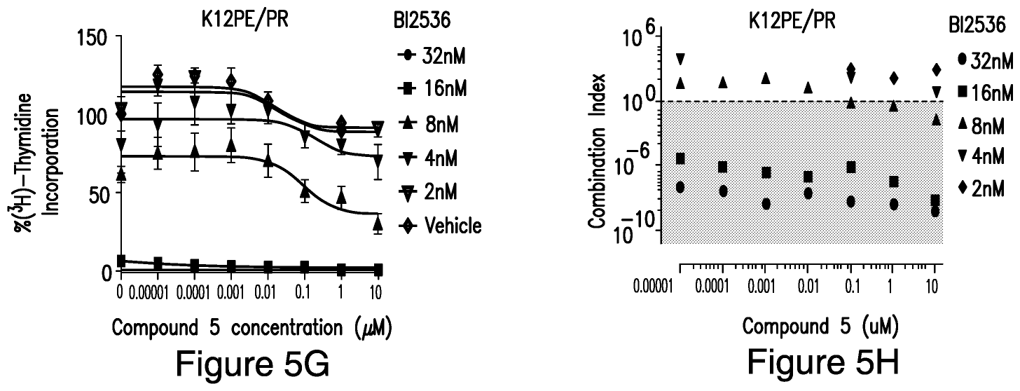

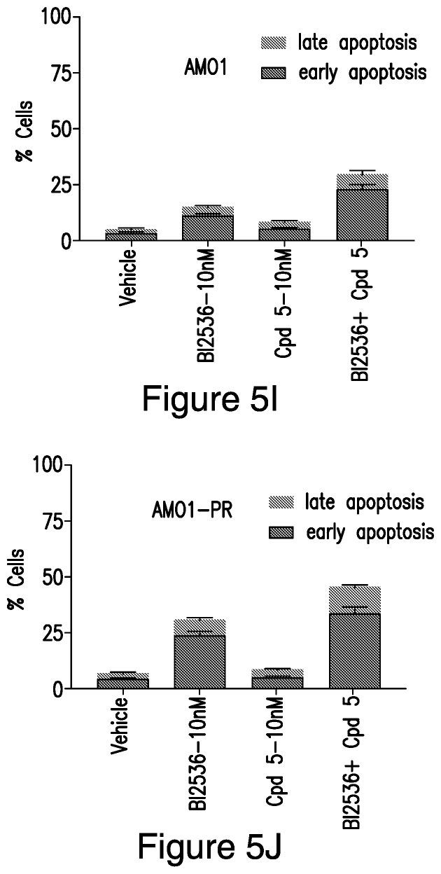

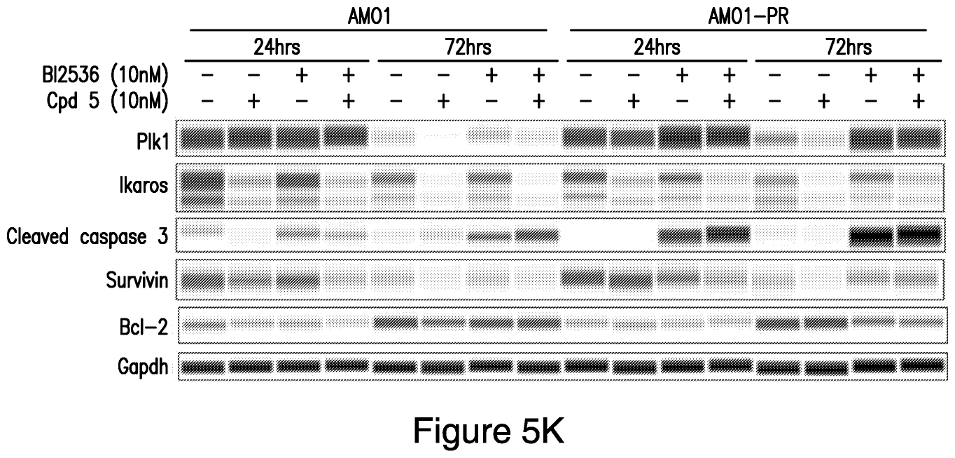

Combination of PLK1 inhibitor with Compound 5 demonstrate superior activity in AMO1-PR cells compared to AMO-1 parental. The PLK1 inhibitor BI2536 and Compound 5 were tested for their activity as individual agents and in combination in AMO1 parental and AMO1-PR cell lines. BI2536 showed a dose dependent decrease in proliferation in combination with Compound 5 (FIG. 5A, FIG. 5C). Synergy analysis using Calcusyn software indicated that the combination treatment was synergistic at several concentrations of BI2536 and Compound 5 (FIG. 5B, FIG. 5D). AMO1-PR cells demonstrated a more dramatic decrease in proliferation in response to BI2536 and several concentrations of BI2536 were synergistic with Compound 5 in these cells. These results suggest a higher dependency of AMO1-PR cells on PLK1 signaling. Another pomalidomide sensitive and resistant cell line pair K12PE and K12PE-PR demonstrated similar synergistic activity of BI2536 and Compound 5 combination (FIG. 5E, FIG. 5F, FIG. 5G, FIG. 5H). Changes in apoptosis were analyzed using the single agent treatments and their combination using Annexin V and Topro staining. Single agent treatment of BI2536 caused a modest increase in early (10.9% vs 2.69%) and late apoptosis (4.25% vs 2.24%) compared to vehicle in AMO-1 cells (FIG. 5I). Compound 5 treatment showed a slight increase in early apoptosis (4.86% vs 2.69%) and almost no effect on late apoptosis (3.07% vs 2.24%) compared to vehicle. Combination treatment of BI2536 and Compound 5 demonstrated a more pronounced increase in early (22.7% vs 2.69%) and late apoptosis (7.09% vs 2.24%) in comparison with vehicle. In case of AMO1-PR cells, BI2536 single agent was more effective than AMO-1 parental cells with changes in early (23.2% vs 3.82%) and late (7.55% vs 2.77%) compared to vehicle. In these cells also, the combination of BI2536 with Compound 5 demonstrated higher early (33.3% vs 3.82%) and late (11.8 vs 2.77%) apoptosis in comparison with vehicle (FIG. 5J). The mechanism of synergy for the BI2536 and Compound 5 treatments was investigated by studying the changes in cell cycle and mitotic fidelity. In AMO1 cells, BI2536 treatment induced a modest increase in G2-M and polyploidy population consistent with the reported mechanism of action of the inhibitor. Compound 5 caused a modest increase in G0-G1 and a decrease in G2-M cells. Combination treatment demonstrated an increase in sub-G1 cells compared to the single agent treatments consistent with the changes in apoptosis. In case of AMO1-PR cells, BI2536 caused a more significant increase in G2-M and polyploidy and sub-G1 cells compared to AMO1 parental cells. Combination of BI2536 and Compound 5 demonstrated a higher increase in sub-G1 cells compared to the individual treatments. Changes in Ikaros and pro-survival signaling in these cell lines was analyzed in response to BI2536 and Compound 5 after 24 and 72 hours of treatment (FIG. 5K). Ikaros levels were decreased in response to Compound 5 in both AMO1 and AMO-1 PR cells. Combination of BI2536 and Compound 5 led to a greater decrease in its levels at 24 hours. Cleaved caspase 3 levels consequently were more significantly increased at 72 hours post combination treatment in both AMO1 and AMO1-PR cell lines. Pro-survival signaling markers, Survivin and Bcl2 demonstrated a greater decrease in BI2536 and Compound 5 combination at 24 hours compared to single agents which could lead to the subsequent enhancement in apoptosis, as evident by cleaved caspase 3 levels. Survivin gene expression significantly correlates with PLK1 expression. Further, confocal imaging to study changes in DAPI staining in AMO1 and AMO1PR cells in response to these treatments suggest higher mitotic errors for BI2536 and BI2536 and Compound 5 combination in these cell lines (data not shown).

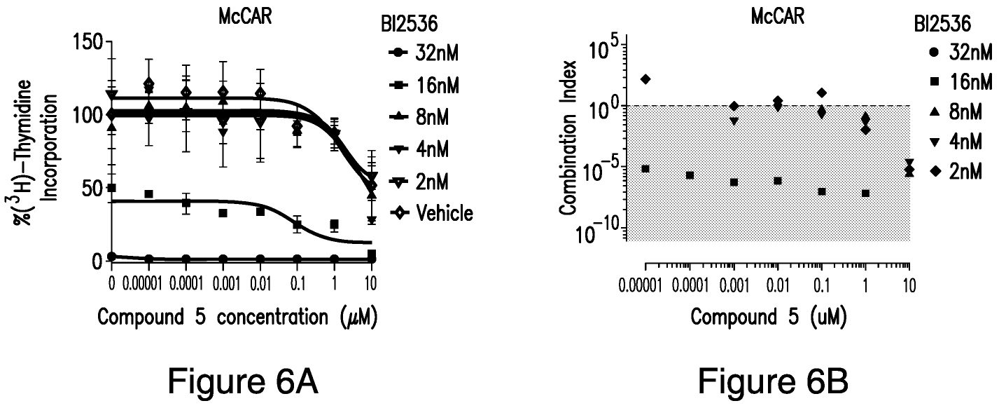

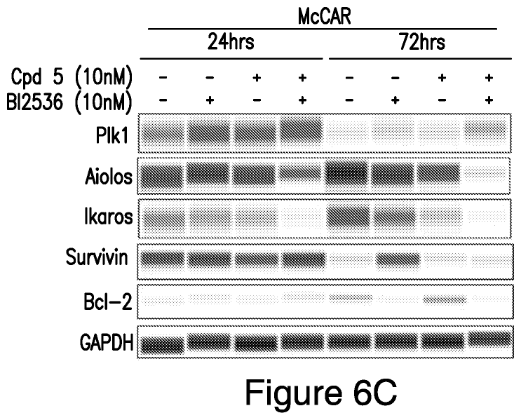

Synergistic cyto-toxicity of BI2536 and Compound 5 combination in refractory cells. Since PLK1 expression was higher in high risk cluster in MGP, the activity of PLK1 inhibitor in combination with Compound 5 was analyzed in a refractory cell line, Mc-CAR. In Mc-CAR cells, BI2536 in combination with Compound 5 demonstrated a synergistic decrease in cell proliferation at various concentrations (FIG. 6A, FIG. 6B). The combination treatment caused a more pronounced decrease in Aiolos and Ikaros levels (FIG. 6C) and consequent increase in sub-G1 arrest compared to individual treatments (data not shown).

PLK1 knock down decreases proliferation and increases apoptosis of AMO1 and AMO1-PR cells. To further ascertain the role of PLK1 in resistance, inducible knock down of PLK1 in AMO1 and AMO1-PR cell lines was generated. Two inducible PLK1 shRNAs demonstrated robust knock down of PLK1 protein in AMO1 and AMO1-PR cell lines and caused a significant decrease in cell proliferation at 48 and 72 hours post induction of knock-down compared to the control shRNA. In both the cell lines, knock-down resulted into G2-M arrest and increase in sub-G1 population at 48 and 72 hours. Analysis of apoptosis further confirmed an increase in apoptosis as a consequence of knock-down using PLK1 shRNAs in both AMO1 and AMO1-PR cell lines, with AMO1-PR cell line demonstrating overall higher apoptosis.

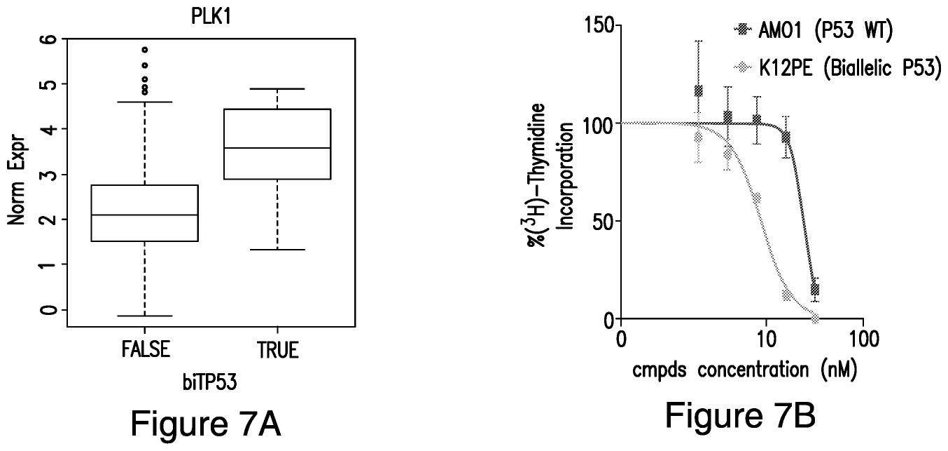

Targeting PLK1 in P53 dysregulated segment. In order to further identify a clinically actionable MM patient segment for PLK1 targeting, the expression of PLK1 in biallelic P53 segment was analyzed, since PLK1 regulates the stability of P53. In MGP, patients who harbored biallelic P53 demonstrated significantly elevated expression of PLK1 (FIG. 7A), indicating an antagonistic relationship of these two proteins. Further, PLK1 inhibitor, BI2536 showed higher activity in biallelic P53 cell line K12PE compared to P53-wild type, AMO1 cells (FIG. 7B), indicating the potential of targeting dysfunctional P53 segment.

### Example 2: BET Inhibition Decreases Cell Proliferation in Multiple Myeloma Cell Lines

Methods.

Patients and datasets. The Myeloma Genome Project (MGP) is a collaborative research initiative to assemble and uniformly analyze genetic datasets that have been generated on samples obtained from patients with MM. Next generation sequencing (NGS) data from patients with NDMM in the MGP dataset were processed and analyzed in a uniform manner as described. Patients with a complete dataset (n=514) from the full MGP dataset (N=1273) including whole exome and genome sequencing (WES and WGS), RNA sequencing (RNAseq), progression free survival (PFS), and overall survival (OS) were used for this analysis. Differences between study designs, data collection, and sequencing approaches resulted in non-uniform availability of all data features for all patients in the MGP dataset.

Cell lines: All MM cell lines (ATCC, Manassas, Va., USA) were routinely tested for Mycoplasma and maintained as previously described in Example 1.

Antibodies: Several antibodies were used for immunoblotting in these experiments including Aiolos (Cat #15103), Ikaros (Cat #14859), BRD4 (Cat #13440), c-Myc (Cat #5605), Cleaved caspase 3 (Cat #9664), Survivin (Cat #2803), GAPDH (Cat #14C10), all from Cell signaling technologies (Danvers, Mass., USA), E2F2 (Cat #Ab-138515, Abcam, Cambridge, Mass., USA), CKS1B (Cat #36-6800, Invitrogen, Waltham, Mass., USA), PRKDC (Cat #4602, Cell signaling, Danvers, Mass., USA), NUP93 (Cat #A303-979A, Bethyl laboratories Montgomery, Tex., USA), RUSC1 (Cat #NBP1-81006, Novus, Saint Charles, Mo., USA), RBL1 (Cat #TA811337, Rockville, Md., USA), NUF2 (Cat #NBP2-43779, Novus Saint Charles, Mo., USA), (TOP2A (Cat #PA5-46814, Invitrogen, Waltham, Mass., USA), K167-FITC (Cat #NBP2-2211F, Novus, Saint Charles, Mo., USA) and Cleaved Caspase 3-AF488 (Cat #IC835G, Minneapolis, Minn., USA).

Proliferation and viability assays: Cell growth curves were determined by monitoring the viability of cells with Trypan blue exclusion on a Vi-Cell-XR (Becton Dickinson, Franklin Lakes, N.J., USA). Proliferation assays were performed in triplicate at least three times (n=3) using (3H)-thymidine incorporation. All data were plotted and analyzed using GraphPad Prism 7 (GraphPad Software, La Jolla, Calif., USA) software, represented as the mean with an error determined as ±s.d.

Immunoblotting: Immunoblot analysis was performed as suggested by WES kits, (Protein Simple, San Jose, Calif., USA) at least two times each (n≥2), where the best representative is shown.

RNA Extraction, Reverse Transcription, and Real-Time PCR Analysis: Total RNA was extracted using a RNeasy plus kit (Qiagen, Germantown, Md., USA) and reverse-transcribed using an iScrip reverse transcription kit (Bio-Rad, Philadelphia, Pa., USA). Quantitative real-time PCR (qPCR) analyses were performed using Taqman PCR Master Mix and the ViiA 7 Real-Time PCR System (Applied Biosystems, Foster City, Calif., USA). Gene expressions were calculated following normalization to GAPDH levels using the comparative CT method (ΔΔCT method). The primer sequences for qPCR are listed in the following table.

List of Primers and their Sequences of MRs and Target Genes Utilized for Transcript Studies

| Oligo sequence (5′ to 3′) | Oligo name | Oligo sequence (5′ to 3′) | Oligo name |
|---|---|---|---|
| ACTGGAAGTGCCCGACAG (SEQ | E2F2 F | ACGGAGAAAGCATGAGCAAT (SEQ ID | BUB1 F |
| ID NO: 9) | | NO: 31) | |
| TCCTCTGGGCACAGGTAGAC | E2F2 R | CAAAAGCATTTGCTTCTTTCCT (SEQ | BUB1 R |
| (SEQ ID NO: 10) | | ID NO: 32) | |
| ATCTTGGCGTTCAGCAGAGT | CKS1B F | GCTGGATCCACCAAAGATGT (SEQ ID | TOP2A F |
| (SEQ ID NO: 11) | | NO: 33) | |
| CGGAACAGCAAGATGTGAGG | CKS1B R | CATGTCCACATAACTACGAAATCC | TOP2A R |
| (SEQ ID NO: 12) | | (SEQ ID NO: 34) | |
| AGCAGTTGGAGCTGTGGTTT | RUSC1 F | CAAGCAGCTTTCAGATGGAAT | NUF2 F |
| (SEQ ID NO: 13) | | (SEQ ID NO: 35) | |
| ATCCTGTTGGCAGGTACAGG | RUSC1 R | GAATTTCCCTCTTGCAGCAC (SEQ ID | NUF2 R |
| (SEQ ID NO: 14) | | NO: 36) | |
| AAGCAGATGGAGCGTGCTAT | MSN F | AGAGAGGCCTGGCTCTGG (SEQ ID NO: 37) | MCM2 F |
| (SEQ ID NO: 15) | | | |
| CCTACGGGTCTGTTCTTCCA | MSN R | CACCACGTACCTTGTGCTTG (SEQ ID | MCM2 R |
| (SEQ ID NO: 16) | | NO: 38) | |
| AGTGCGCATGGAGTTTGTC (SEQ | NUP93 F | CAGAAGAAGAAAGAAAAATTGGAGA | HELLS F |
| ID NO: 17) | | (SEQ ID NO: 39) | |
| TGCAAATTTCCAAAGACAGTCA | NUP93 R | TGGCTTCTCTTCACTTGCAT (SEQ ID | HELLS R |
| (SEQ ID NO: 18) | | NO: 40) | |
| TCGCACCTTACTCTGTTGAAA | PRKDC F | GGAGTTATACCCAGGGCAAT (SEQ ID | CENPE F |
| (SEQ ID NO: 19) | | NO: 41) | |
| CCAGGGCTGGAATTTTACAT | PRKDC R | CACGTAAGAGAAATTCCCTATCA | CENPE R |
| (SEQ ID NO: 20) | | (SEQ ID NO: 42) | |
| GGTCAGACAGCCCAGATGTT | KIF4A F | GATCAGCAGGACACCCAGAT (SEQ ID | MCM3 F |
| (SEQ ID NO: 21) | | NO: 43) | |
| GCTCTTCTAGCTTGGCGTTC | KIF4A R | TGCTGCACTCACCATCTTCT (SEQ ID | MCM3 R |
| (SEQ ID NO: 22) | | NO: 44) | |
| CGAAAGCATCCTTCATCTCC | TPX2 F | GAACCACCAAAGTTACCACGA (SEQ | RBL1 F |
| (SEQ ID NO: 23) | | ID NO: 45) | |
| TCCTTGGGACAGGTTGAAAG | TPX2 R | TCCAACAGAAATTAAACAGATCCTT | RBL1 R |
| (SEQ ID NO: 24) | | (SEQ ID NO: 46) | |
| GGTCAGACAGCCCAGATGTT | KIF4A F | GCAAAAACGAAAGCAACTGG | NONO F |
| (SEQ ID NO: 25) | | (SEQ ID NO: 47) | |
| GCTCTTCTAGCTTGGCGTTC | KIF4A R | CGCATCATTTCTTCTTGCTG (SEQ ID | NONO R |
| (SEQ ID NO: 26) | | NO: 48) | |
| ATGAGAAACCTGAATCCAGAAG | MCM4 F | ATCGAGCGAACGCTTTACTT (SEQ ID | POLA1 F |
| (SEQ ID NO: 27) | | NO: 49) | |
| ATCAGCTGGGATGTCCTGAT | MCM4 R | TTCCTGTTTCTTTCCCCGTA (SEQ ID | POLA1 R |
| (SEQ ID NO: 28) | | NO: 50) | |
| GCGTCACTGGTATTTTCTTGC | MCM7 F | GACGCGAGAACTTCCAGAAC (SEQ ID | TAGLN2 F |
| (SEQ ID NO: 29) | | NO: 51) | |
| ATGGGCTTCCAGGTAGGTTT | MCM7 R | CCTCGGGGTACAGTGCATTA (SEQ ID | TAGLN2 R |
| (SEQ ID NO: 30) | | NO: 52) | |

ChIP-seq studies: ChIP-sequence experiments in DF15, MM1.S and AMO1 cell lines were performed using standard methods.

Flow cytometry: Annexin-V Alexa Fluor 488-conjugated antibody (Thermo Scientific, Waltham, Mass., USA) and To-Pro-3 (Thermo Scientific, Waltham, Mass., USA) were used according to the manufacturer's protocol and processed using Flow Jo software for at least three independent experiments. For cell cycle analysis, cells were stained with PI staining kit (Abcam, Cambridge, Mass., USA) and analysis was performed on Flow Jo V10 software.

Confocal Imaging: Cells were cultured in chambered slides and fixed permeabilized for microscopy. Cells were incubated with primary antibodies targeting CKS1B, E2F2, K167-FITC in 1× intracellular staining buffer for 2 hours in cold room. Cells were then stained with Alexa Flour 488 and 594 conjugated secondary antibodies for 30 mins at RT for CKS1B and E2F2, washed and counterstained with DAPI. Confocal images were captured using Nikon A1R (Melville, N.Y., USA).

shRNA knockdown: Doxycycline (DOX)-inducible shRNA constructs targeting CKS1B, E2F2 and BRD4 were generated by Cellecta (Mountain View, Calif., USA) using pRSITEP-U6Tet-(sh)-EF1-TetRep-2A-Puro plasmid. Luciferase negative control were generated as previously described (PMID: 21189262). Briefly, 293T cells were co-transfected with lentiviral packaging plasmid mix (Cellecta, Cat #CPCP-K2A) and pRSITEP-shRNA constructs. Viral particle was collected 48 after transfection and then concentrated 10-fold by Amicon Ultra-15 centrifugal filters. For infections, cells were incubated overnight with concentrated viral supernatants in the presence of 8 μg/ml polybrene. Cells were then washed to remove polybrene. At 48 hours post-infection, cells were selected with puromycin (1 μg/ml) for more than 3 weeks before experiments. The shRNA target sequences were:

CKS1B shRNA1:
(SEQ ID NO: 53)
5′ GACCCACAGCCTAAGCTGAGT 3′;
E2F2 shRNA2:
(SEQ ID NO: 54)
5′ GTACGGGTGAGGAGTGGATAA 3′,
BRD4 shRNA1:
(SEQ ID NO: 55)
5′ GACGTGGGAGGAAAGAAACAG 3′,
BRD4 shRNA2:
(SEQ ID NO: 56)
5′ GTGCTGACGTCCGATTGATGT 3′,
BRD4 shRNA3:
(SEQ ID NO: 57)
5′ CGCAAGCTCCAGGATGTGTTC 3′,
BRD4 shRNA4:
(SEQ ID NO: 58)
5′ GCTCCTCTGACAGCGAAGACT 3′.

Results.

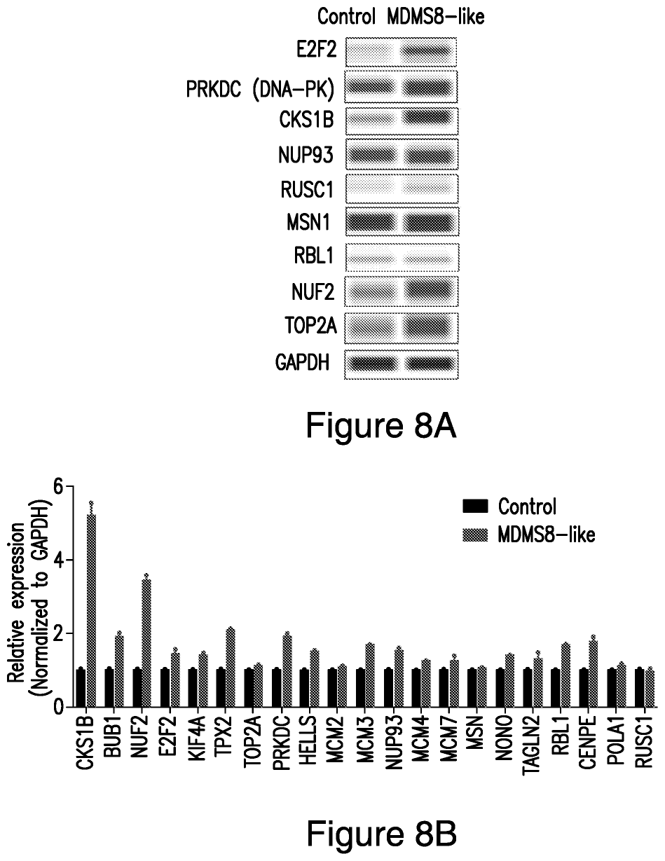

Expression of MRs in MDMS8-like cells. The identification of the MRs provided an opportunity to explore their role in high-risk MM biology. An enrichment score based on the MDMS8 gene signature across a panel of myeloma cell lines was run to infer activation of this signature in the samples. This approached identified various cell lines which presented significant association with MDMS8's GE phenotype. One MDMS8-like cell line (DF15PR) and a non-MDMS8-like one (MM1.S) were selected as controls for further functional experiments. qRT-PCR and western blot experiments showed that two of the MRs (E2F2 and CKS1B) and downstream genes (including TOP2A and NUF2) were up-regulated at the protein and transcript expression levels in the MDMS8-like cell line versus a control cell line (FIG. 8A and FIG. 8B). CKS1B and E2F2 showed significant correlation with the expression of their target genes, NUF2 and TOP2A in MGP (data not shown). MDMS8-like cells proliferated faster and had a mean doubling time of approximately 12.55±0.8 hrs vs 17.6±2.2 hrs (P<0.05) in the control cell line. In asynchronous cell culture, analysis of the distribution of cell cycle stages between the MDMS8-like versus control cell line showed an increased percentage of cells in S1 (16.9% vs 8.14%) and G2/M (23.5% vs 17%), with a concomitant decrease in the sub-G1 fraction (1.1% vs 7.7%) respectively, demonstrating hyperproliferative behavior.

MDMS8 GE Phenotype at the Single Cell Level. To further understand the high-risk phenotype and the function of the MRs single-cell gene expression profiling was used to explore whether MDMS8 MR regulons were expressed globally or in a subset of tumor cells. For both control and MDMS8-like cell line transcriptional analysis were performed using the 10× single cell gene expression platform. Asynchronously grown control and MDMS8 cell line was checked, followed by analysis of the E2F2 and CKS1B regulons, and the MDMS8 GE signature activity in each cell. tSNE plots (data not shown) showed cells enriched in MDMS8 signature, and this analysis demonstrated that not all the cells in the MDMS8-like cell line were positive for this phenotype, suggesting that MR activity was restricted to a fraction of the overall population of cells. Active cells (those with MDMS8's phenotype) were selected based on empiric thresholds and a higher subset of them appeared in the MDMS8-like cell line compared to the control one (>40% vs <20% respectively). These findings also indicated that two MRs, CKS1B and E2F2 perhaps are more important in controlling the cell cycle profile of MDMS8-like cells (data not shown).

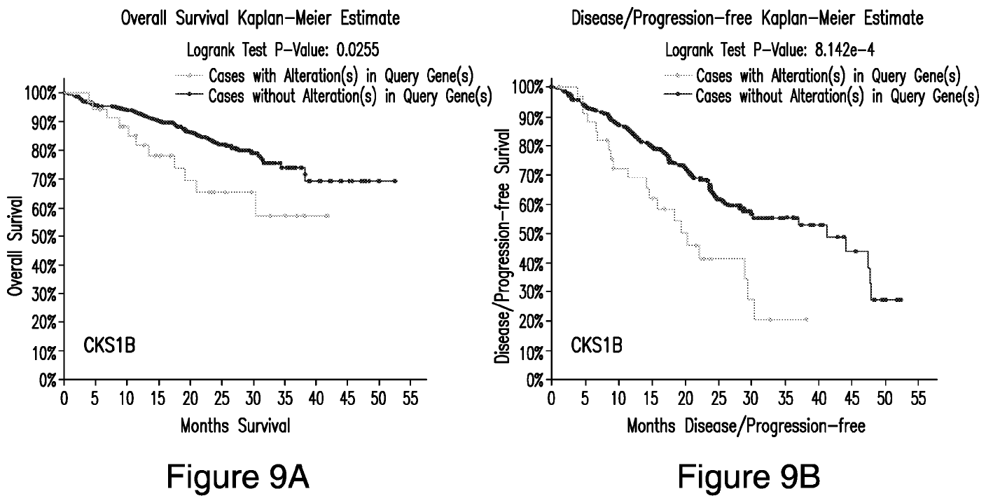

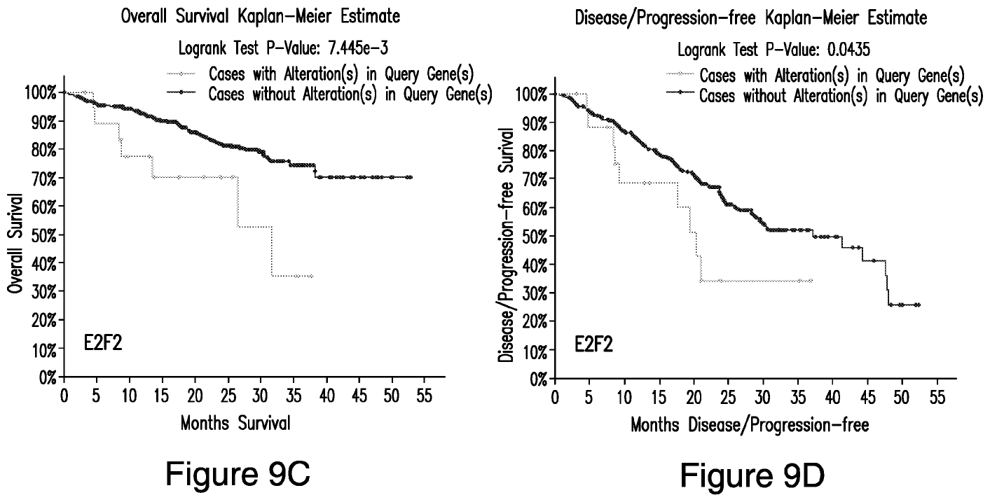

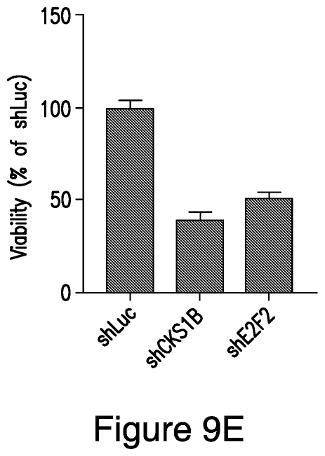

Prognostic and functional role of CKS1B and E2F2. The association of CKS1B and E2F2 expression on overall and progression free survival (OS, PFS) was analyzed in patients from MGP and it was observed that their higher expression significantly correlated with lower OS and PFS (FIG. 9A, FIG. 9B, FIG. 9C, and FIG. 9D). shRNA cell lines were established to perform knock-down studies of CKS1B and E2F2. MDMS8-like cells upon knock-down of CKS1B and E2F2 demonstrated a significant decrease in proliferation and increase in apoptosis (FIG. 9E), suggesting the functional role of these two MRs in viability of these cells.

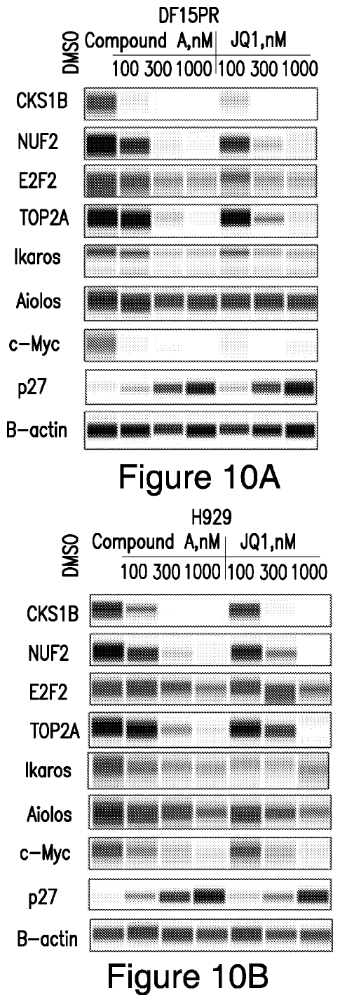

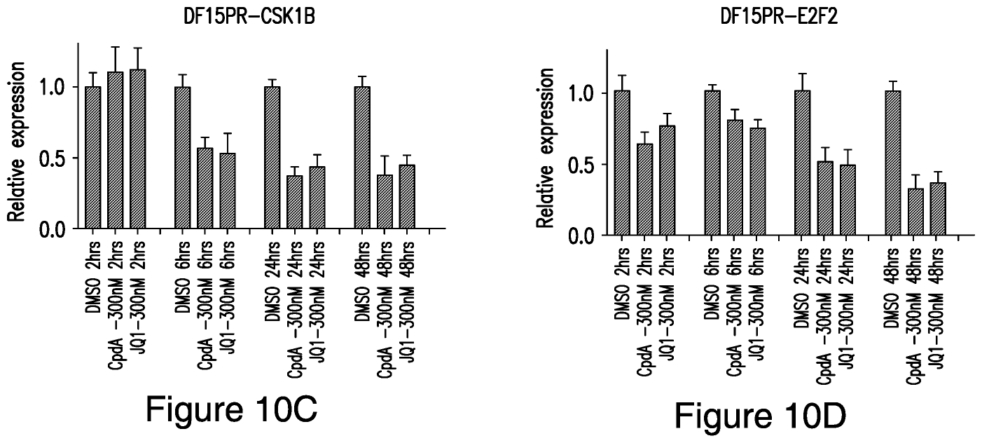

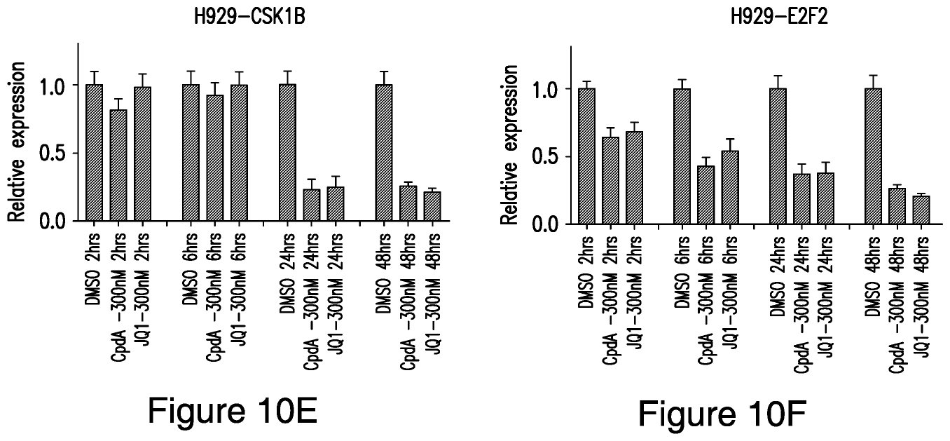

Effects of BRD4 inhibitors on CKS1B and E2F2 and their target genes. CKS1B and E2F2 have been enlisted as super-enhancer (SE) associated genes in MM (Loven, J., et al., Cell, 2013, 153(2): p. 320-34). In order to pharmacologically target CKS1B and E2F2, the BET inhibitors JQ1 and Compound A were utilized in MDMS8-like and H929 cell lines. Both JQ1 and Compound A demonstrated a dose and time dependent decrease in the protein levels of CKS1B and E2F2 (FIG. 10A and FIG. 10B). As a surrogate for activity, protein expression of their target genes NUF2 and TOP2A, respectively for CKS1B and E2F2 also decreased. BET inhibitors also promoted a decrease in Cereblon substrates, Ikaros, Aiolos and c-Myc levels. Further, an increase in the levels of P27 was observed, which is a negative regulator of CKS1B signaling. Immunofluorescence staining was performed to analyze the localization and expression of CKS1B and E2F2 in response to JQ1 and confirmed the decrease in their nuclear expression in MDMS8-like cells (data not shown). Since BET inhibitors mediate their changes mainly at transcript levels, transcript levels of CKS1B and E2F2 were analyzed in response to BET inhibitors. In both MDMS8-like and H929 cell lines, BET inhibitors promoted a decrease in transcript levels of CKS1B and E2F2 (FIG. 10C, FIG. 10D, FIG. 10E, and FIG. 10F). The expression of NUF2, TOP2A, Ikaros and Aiolos were also downregulated at transcript levels in response to BET inhibitors (data not shown). To ascertain the SE mediated regulation of CKS1B and E2F2, CDK7 inhibitors targeting SE-associated complexes in MM cell lines were utilized. A CDK7 inhibitor, THZ1, showed a potent decrease in proliferation in several MM cell lines by downregulating CKS1B, E2F2, Myc, Aiolos and Ikaros (data not shown).

Binding of BRD4 on SE-associated regions on CKS1B and E2F2. Using BRD4-ChIP-Seq data in AMO1 and MM1.S cell lines, the binding of BRD4 on the SE-associated regions on CKS1B and E2F2 was analyzed. Robust binding of BRD4 on the SE-associated regions on CKS1B and E2F2 was observed, and in response to JQ1, the binding was abolished in both the cell lines (data not shown).

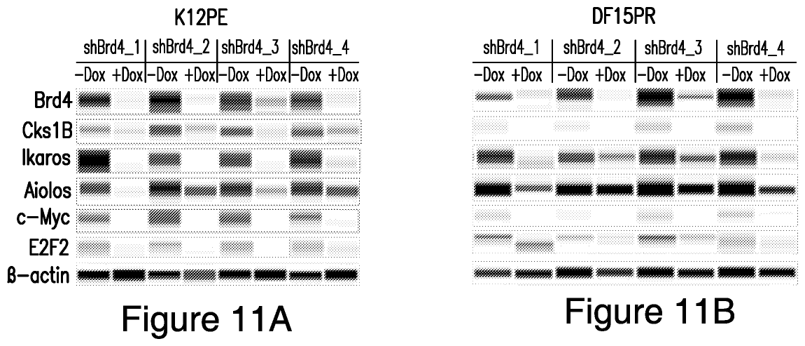

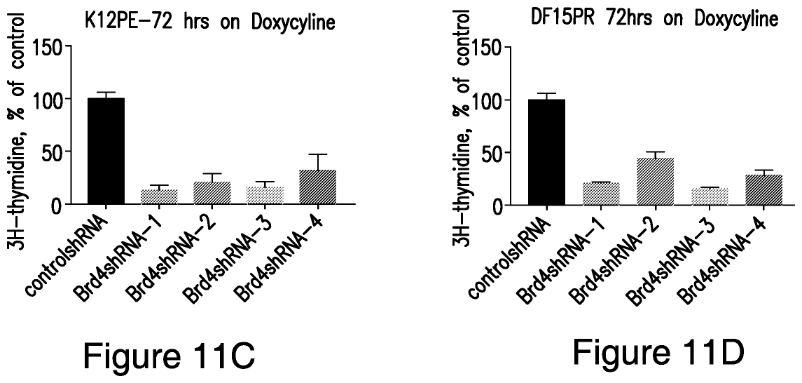

Effects of BRD4 knock-down on CKS1B and E2F2 expression. Doxycycline inducible BRD4 knock-down cell lines in the background of K12PE and MDMS8-like cells were established. Four different shRNA targeting BRD4 in these two cell lines consistently demonstrated a decrease in CKS1B and E2F2 levels (FIG. 11A, FIG. 11B). BRD4 knock-down also resulted into a decrease in Aiolos, Ikaros and c-Myc levels consistent with the findings of BRD4 inhibitors. Changes in cell proliferation, apoptosis and cell cycle in response to BRD4 knock-down were also analyzed. All the four shRNAs in K12PE and MDMS8-like cells caused a marked decrease in cell proliferation (FIG. 11C, FIG. 11D). As a result of knock-down, apoptosis and cell cycle assays indicated an increase in apoptosis and decrease in proportion of cells in G2-M and increase in sub-G1 phases of cell cycle (data not shown).

BRD4 inhibition in 1q amplified MM cell lines. CKS1B is localized on 1q 21.3 and 1q amplification is a high risk segment in MM. Analysis of the activity of BRD4 inhibition in several 1q cell lines harboring 1q amplification (U266, MM1.S, MDMS8-like, H929, KMS11) compared to non-1q amplified cell line (MC-CAR) was performed. As shown in the table below, BRD4 inhibitors were observed to be two-five times more potent in 1q amplified cell lines compared to the non-1q amplified cell line.

| | 1q amp | JQ1 | Compound |
|---|---|---|---|
| MM cell lines | cell lines | IC₅₀ (μM) | A IC₅₀ (μM) |
| McCAR | normal | 0.08352 | 0.09344 |
| U266 | 3× | 0.01482 | 0.03394 |
| MM1.S | 3× | 0.01521 | 0.02815 |
| DF15/PR | 3× | 0.03782 | 0.03589 |
| H929 | 3-4× | 0.05167 | 0.05407 |
| KMS11 | 6-8× | 0.03618 | 0.04035 |

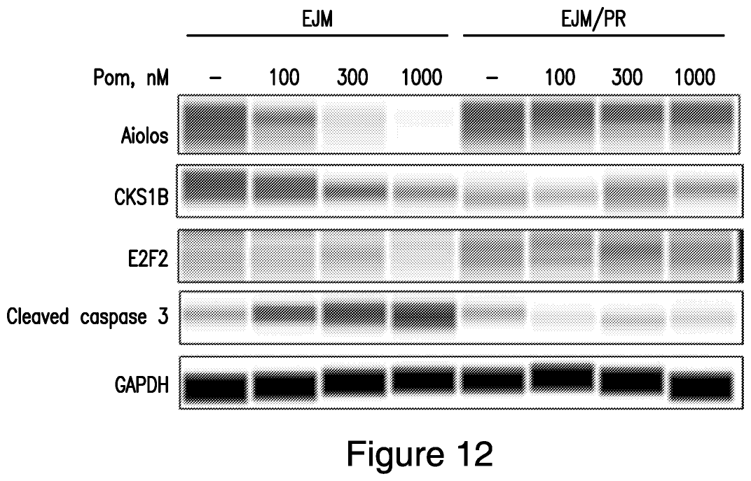

Effects of Pomalidomide (Pom) on CKS1B and E2F2 in Pom sensitive and resistant cell lines. CKS1B and E2F2 are reported to be involved in cell cycle mainly via regulation of P27 and RB-CDK4-CDK6-CCND1 signaling pathways, respectively, and immune-modulatory compounds have demonstrated cell cycle effects by promoting G1 arrest in MM cell lines. Based on these reports, the changes in CKS1B and E2F2 in response to Pom in isogenic Pom sensitive and resistant EJM and EJM-PR cell lines was analyzed and it was found that these two proteins were downregulated at the transcriptional level only in Pom sensitive cells (FIG. 12). Since Aiolos was not degraded in the EJM-PR cell line consistent with the absence of CKS1B and E2F2 downregulation, the binding of Aiolos on the transcriptional start sites (TSS) of CKS1B and E2F2 was analyzed. ChIP-seq data in DF15 cell line indicated the binding of Aiolos on TSS of CKS1B and E2F2 with H3K27Ac activation mark (supportive data of GM12878 cell line from Encode project), suggesting, the role of these two proteins downstream of Aiolos (data not shown). The effects of BRD4 inhibitors on four isogenic Pom sensitive and resistant cell line pairs (K12PE, K12PE-PR, AMO1, AMO1-PR, H929, H929-PR, DF15, DF15-PR) was analyzed and showed that irrespective of their resistance to Pom, these cell lines were equally sensitive to BRD4 inhibitors (data not shown).

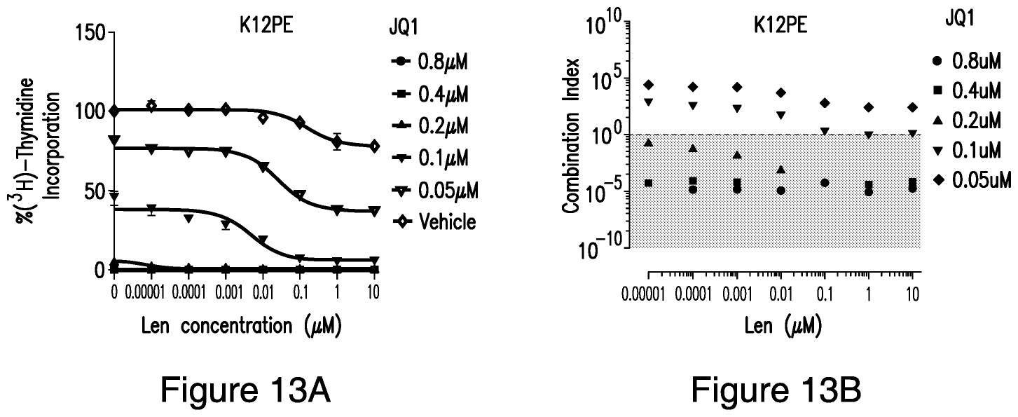

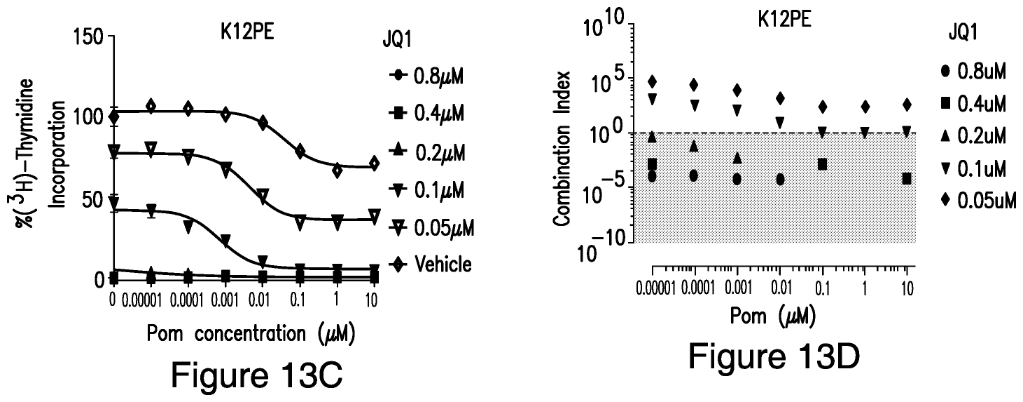

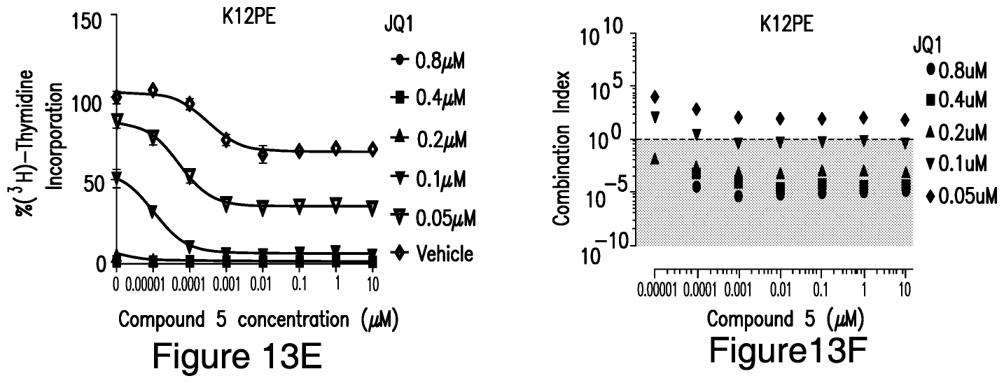

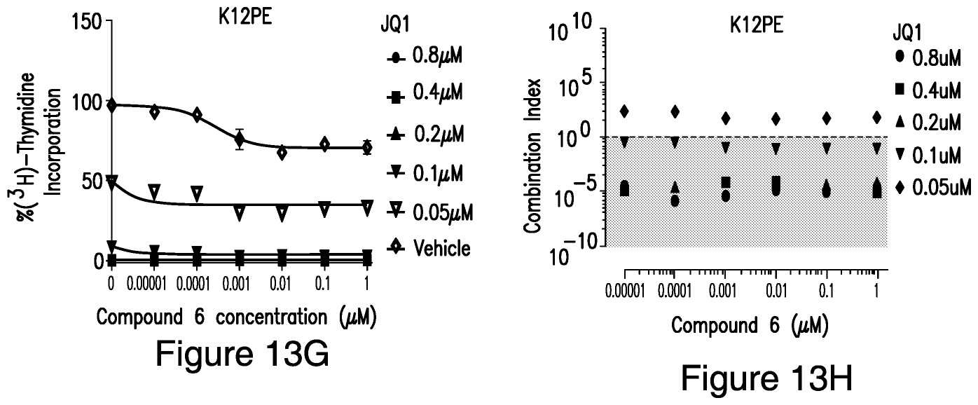

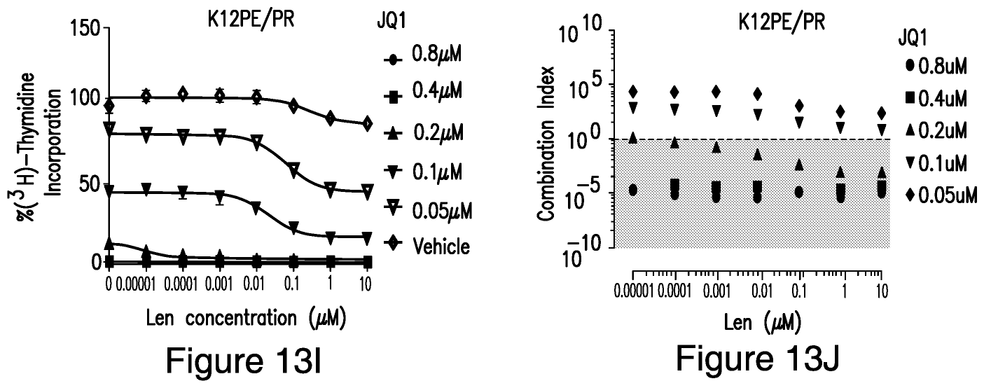

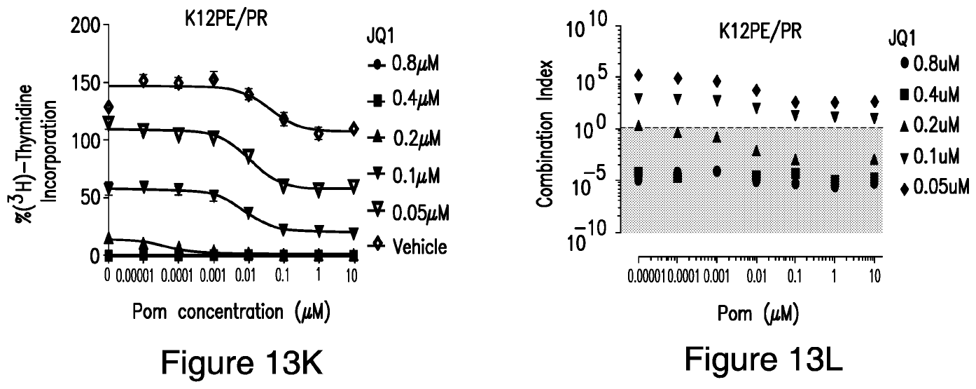

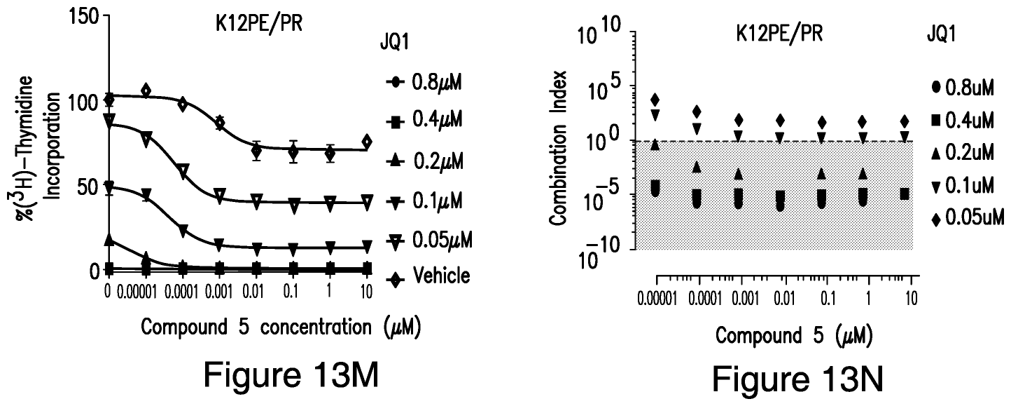

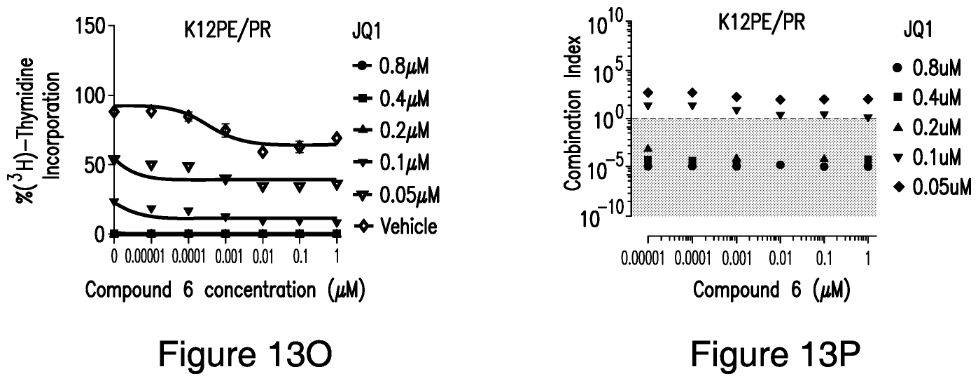

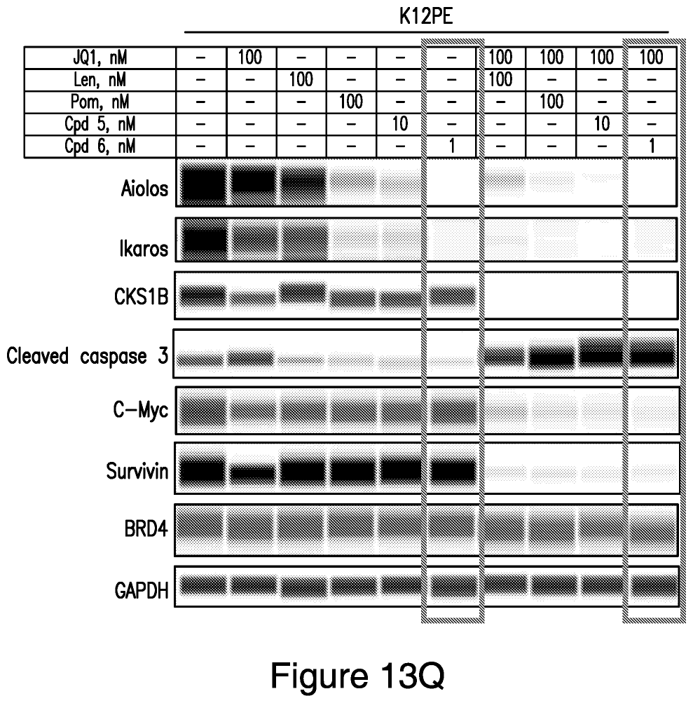

Combinatorial activity of BRD4 inhibitor and antiproliferative compounds. Based on the activity of BRD4 inhibitors and Pom on CKS1B, E2F2 and Cereblon substrates, the changes in proliferation by combining BRD4 inhibitor with the compounds was investigated. JQ1 showed a dose dependent decrease in proliferation in combination with Len, Pom, Compound 5 and Compound 6 (FIG. 13A, FIG. 13C, FIG. 13E, FIG. 13G) in K12PE cells. Synergy analysis using Calcusyn software indicated that the combination treatment was synergistic at several concentrations of JQ1 and Len, Pom, Compound 5 and Compound 6 (FIG. 13B, FIG. 13D, FIG. 13F, FIG. 13H). The combination also synergistically decreased proliferation in Pom resistant, K12PE-PR cell line (FIG. 13I to FIG. 13P). The changes in the signaling in response to the combination treatments of BRD4 inhibitor with Len, Pom, Compound 5 and Compound 6 was analyzed. Combination of JQ1 with Len, Pom, Compound 5 and Compound 6 caused a more profound decrease in the levels of Aiolos, Ikaros, CKS1B, E2F2, Myc, Survivin and a higher increase in cleaved caspase 3 in the combination treatment compared to the monotherapies (FIG. 13Q). Furthermore, cell cycle and apoptosis assays confirmed a more significant decrease in G2-M and increase in apoptosis in the combination treatment of BRD4 inhibitor with Len, Pom, Compound 5 and Compound 6 compared to monotherapies (data not shown).

### Example 3: NEK2 Inhibition Decreases Cell Proliferation in Multiple Myeloma Cell Lines

Cell lines. Cell lines used in this study are AMO1, H929, K12PE, MMIS, purchased from ATCC, USA. Cells were cultured in RPMI 1640 medium supplemented with L-glutamine, sodium pyruvate, fetal bovine serum, penicillin, and streptomycin (all from Invitrogen). Pomalidomide resistant cell lines of AMO1, H929, K12PE, MMIS were generated as previously described (Bjorklund et al., J Biol Chem. 2011, 286(13):11009-11020).

NEK 2 inhibitors. Two inhibitors of NEK2—irreversible inhibitor JH295 and reversible inhibitor rac-CCT 250863 (Tocris Bioscience) were used. Both JH295 and rac-CCT 250863 are selective inhibitors of NEK2, and have low effect on other kinases, including Cdk1 and Aurora B. Additionally, JH295 and rac-CCT 250863 do not affect PLK1, the bipolar spindle assembly, or the spindle assembly checkpoint. (Henise et al., J Med Chem. 2011, 54(12):4133-4146; Innocenti et al., J Med Chem. 2012, 55(7):3228-3241).

Antibodies. Antibodies were used for immunoblotting and flow cytometry in this example. The antibodies used were: NEK2 (Santa Cruz Biotechnologies, Cat #55601,), Aiolos (Cell Signaling Technologies, Cat #15103), Ikaros (Cell Signaling Technologies, Cat #14859), ZFP91 (inhouse antibody), GAPDH (Cell Signaling Technologies, Cat #2118).

Confocal Imaging. Cells were cultured in chambered slides and fixed permeabilized for microscopy. Cells were incubated with primary antibody specific for NEK2 in 1× intracellular staining buffer for 2 hours in cold room. Cells were then stained with Alexa Flour 488 conjugated secondary antibodies for 30 mins at RT, washed and counterstained with DAPI. Confocal images were captured using Nikon A1R (Melville, N.Y., USA).

Proliferation and viability assay. Cell growth curves were determined by monitoring the viability of cells with Trypan blue exclusion on a Vi-Cell-XR (Becton Dickinson, Franklin Lakes, N.J., USA). Cell lines were plated in triplicate in 96-well round-bottom plates with the indicated drug concentrations or knockdown cells. Proliferation assays were performed in triplicate at least three times (n=3) using either the WST-1 tetrazolium salt (Roche Applied Science) reagent used according to the manufacturer's specifications or by (3H)-thymidine incorporation as previously described (Bjorklund et al., Blood Cancer Journal 5, e354, 2015). All data were plotted and analyzed using GraphPad Prism 7 (GraphPad Software, La Jolla, Calif., USA) software, represented as the mean with an error determined as ±s.d.

Immunoblotting. Immunoblot analysis was performed as suggested by WES kits, (Protein Simple, San, CA, USA) at least two times each (n≥2), where the best representative is shown.

Flow cytometry. Annexin-V Alexa Fluor 488-conjugated antibody (Thermo Scientific, Waltham, Mass., USA) and To-Pro-3 (Thermo Scientific, Waltham, Mass., USA) were used according to the manufacturer's protocol and processed as previously described using Flow Jo software for at least three independent experiments. For cell cycle analysis, cells were stained with Propidium Iodide (PI) staining kit (Abcam, Cambridge, Mass., USA) and analysis was performed on Flow Jo V10 software.

Biparametric assay for detection of cell cycle and apoptosis. Cell viability assay with Annexin V-FITC and propidium iodide was performed according to the published protocol cycle (Rieger et al., J Vis Exp. 2011, (50):2597; Léonce et al., Mol Pharmacol. 2001, 60(6):1383-1391).

Results:

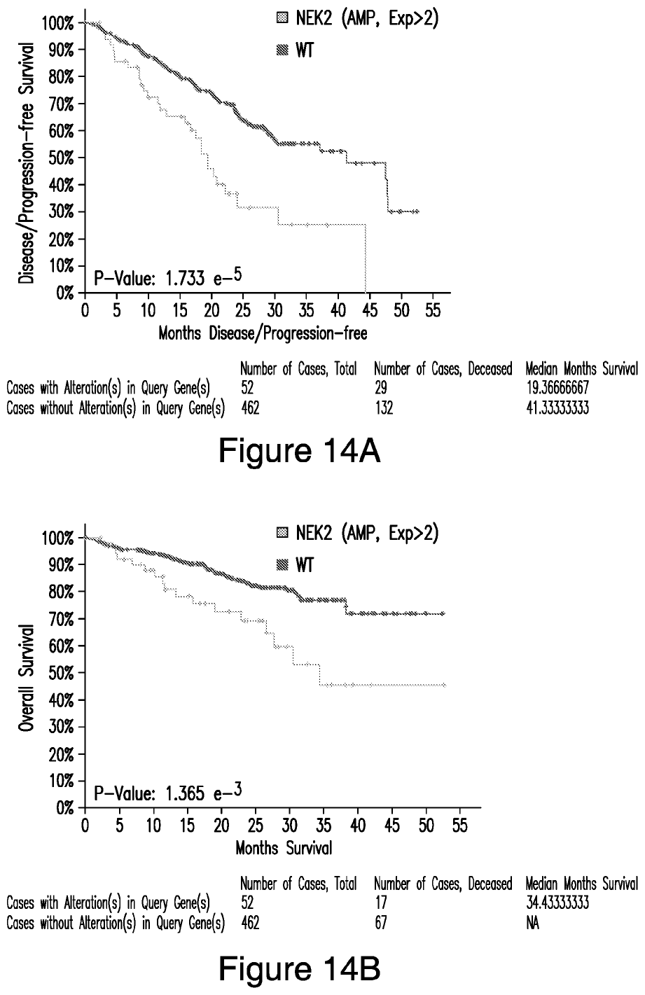

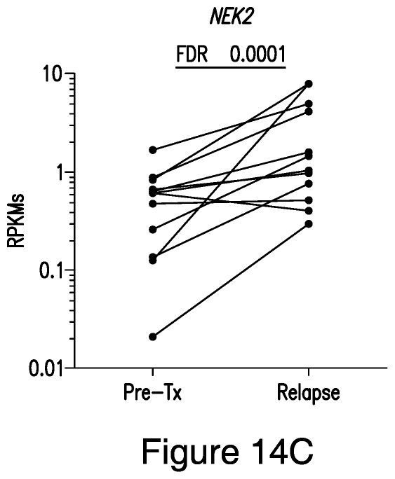

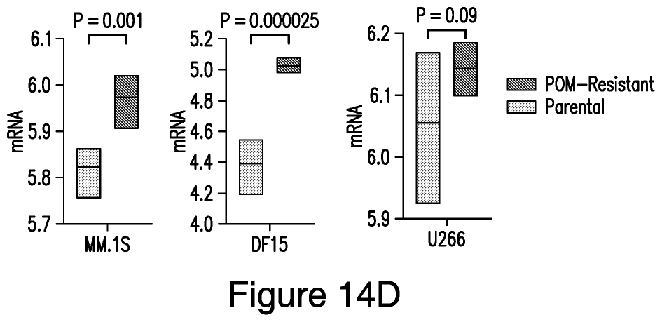

NEK2 upregulation is associated with high risk disease and relapse in MM patients. A molecular classification for newly diagnosed multiple myeloma (ndMM) was generated that classified ndMM into 12 distinct molecularly defined disease segments (MDMS 1-12). This integrative analysis identified a molecularly defined disease segment 8 (MDMS8) as high-risk cluster with poorest clinical outcome. Further analysis of MDMS8 revealed upregulation of several chromosomal instability (CIN) genes. Aberrant expression of one particular CIN gene, NEK2 was found in about 10% of ndMM population. Higher NEK2 expression was significantly associated with lower progression free and overall survival (P-value 1.733 e^{-0.5} and 1.365 e^{-0.3}, respectively (FIG. 14A, FIG. 14B). NEK2 expression was assessed in 12 paired sample from a Lenalidomide based trial. Nek2 expression was measured in treatment naïve and relapsed samples using RNA seq and it was found that NEK2 expression is significantly increased upon disease relapse (FDR<0.0001, FIG. 14C). Increased NEK2 expression has previously been reported to be associated with drug resistance and relapse (Zhou et al., Cancer Cell 23(1), p48-62, 2013). To further confirm this MM1S, DF15 and U266 pomalidomide-resistant cell lines were generated by continued drug exposure. RNA seq analysis of isogenic drug sensitive and drug resistant cell line pairs showed significant upregulation of NEK2 expression in drug resistant cell lines compared to the drug sensitive counterparts (FIG. 14D). Immunocytochemistry combined with confocal microscopy also showed increased expression of NEK2 in the nucleus in resistant myeloma cell line compared to parental cell line (data not shown). These findings show that increased NEK2 expression is associated with poor prognosis, acquired drug resistance, and disease relapse.

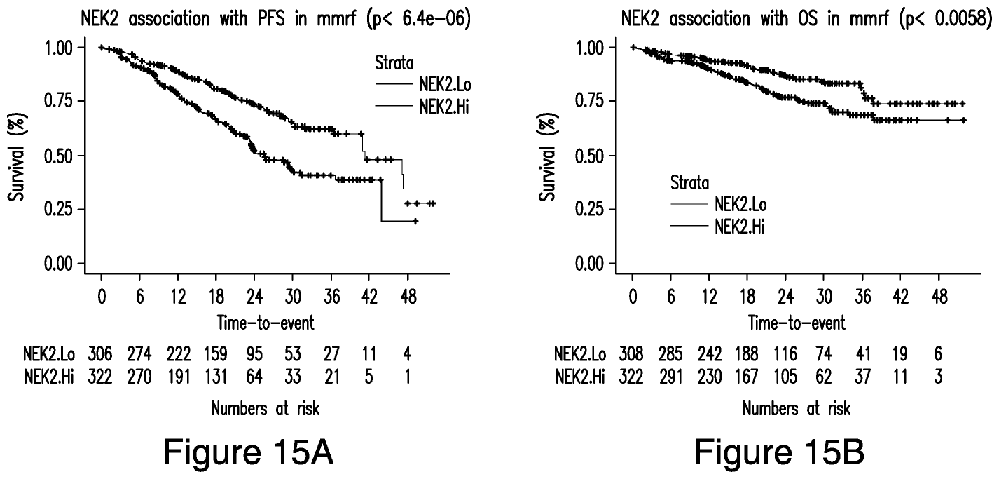

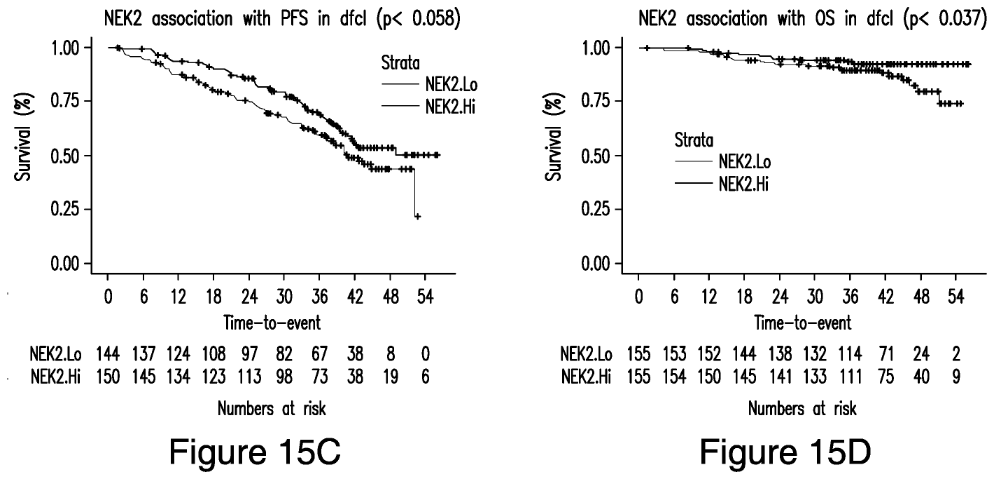

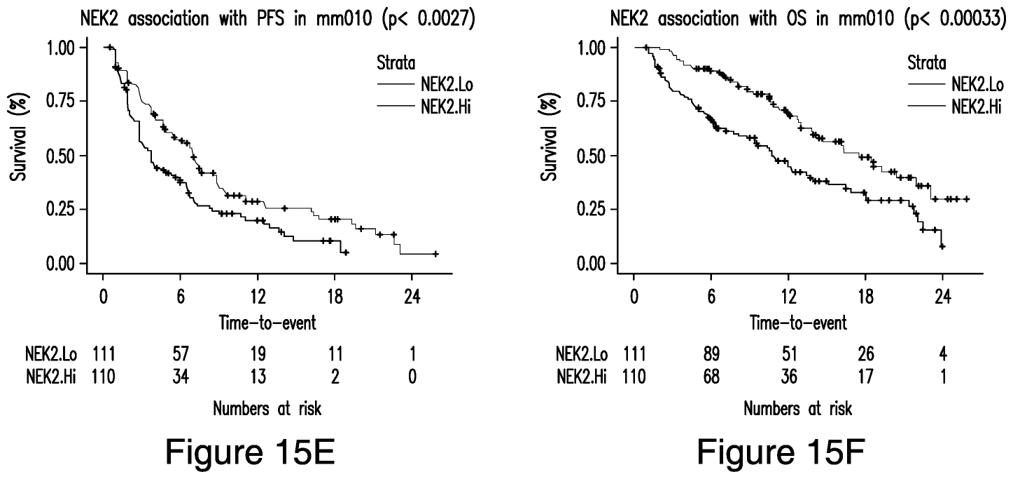

To further validate the association between elevated NEK2 expression and poor survival, Kaplan Meier analysis was performed in additional myeloma datasets: newly diagnosed MMRF and newly diagnosed DFCI and relapsed refractory MM0010 datasets. Elevated NEK2 expression was significantly associated with poor PFS (FIG. 15A and FIG. 15E; P-value<6.4e^{-0.6} and 0.0027, in ND MMRF and MM0010, respectively), and OS (FIG. 15B and FIG. 15F; P-value<0.0058 and 0.00033, in ND MMRF and MM0010, respectively) in newly diagnosed MMRF and relapsed refractory MM0010 datasets. Elevated NEK2 expression also shows a poor PFS and OS in DFCI datasets but it was not statistically significant (FIG. 15C and FIG. 15D).

NEK2 inhibition decreases cell proliferation in MM cell lines. To test the functional role of NEK in myeloma biology the effects of NEK2 chemical inhibition on MM cell proliferation in the presence of irreversible inhibitor JH295 (Henise et al., J Med Chem. 2011, 54(12):4133-4146) and reversible inhibitor Rac-CCT 250863 (Innocenti et al., J Med Chem. 2012, 55(7):3228-3241) were analyzed. A strong antiproliferative effect of NEK2 inhibition on multiple myeloma cell lines (H929, AMO1, K12PE and MC-CAR) was observed. The IC₅₀ concentrations of JH295 were 0.37 μM, 0.48 μM, 4 μM and 0.56 μM, respectively, for H929, AMO1, K12PE and MC-CAR cell lines at Day 3 post treatment. The IC₅₀ concentrations of Rac-CCT 250863 were 8.0 μM, 7.1 μM and 8.7 μM, respectively, for H929, AMO1 and K12PE cell lines at Day 3 post treatment.

NEK2 inhibitors decreased proliferation in both pomalidomide sensitive and resistant cell lines. It was found that higher NEK2 expression is associated with acquired drug resistance (FIG. 14D). The effect of NEK2 inhibition in pomalidomide resistant cell lines was assessed through treatment of three isogenic pomalidomide sensitive and resistant (PR) cell lines: H929, H929-PR, AMO1, AMO1-PR, K12PE, K12PE-PR with increasing concentrations of JH295 and Rac-CCT 250863 inhibitors. The effects of JH295 and Rac-CCT 250863 inhibitors on proliferation were analyzed. Both NEK2 inhibitors decreased proliferation in pomalidomide sensitive and resistant cell lines. The IC₅₀ concentrations of JH295 were 0.37 μM, 0.27 μM, 0.48 μM, 0.31 μM, 4.00 μM, and 10.8 μM, respectively, for H929, H929-PR, AMO1, AMO1-PR, K12PE, and K12PE-PR cell lines. The IC₅₀ concentrations of Rac-CCT 250863 were 7.90 μM, 5.20 μM, 7.00 μM, 3.60 μM, 8.50 μM, and 5.17 μM, respectively, for H929, H929-PR, AMO1, AMO1-PR, K12PE, and K12PE-PR cell lines. JH295 was more effective than Rac-CCT 250863 in pomalidomide resistant cell lines and JH295 was more effective in decreasing proliferation of H929-PR and AMO1-PR cell lines compared to their parental counterparts. This shows a higher vulnerability of drug resistance lines on NEK2 inhibition. Lower IC₅₀ values of NEK2 inhibitors in H929 PR and AMO1 PR in comparison to H929 and AMO1 cell lines demonstrated increased sensitivity to NEK2 inhibitors in the resistant cell lines, illustrating increased dependency of drug resistant lines on NEK2 expression.

NEK2 knock-down decreases cell proliferation of drug sensitive and resistant MM cell lines. To study the effects of NEK2 knock-down on MM cell proliferation, tetracycline inducible NEK2 shRNA cell lines were established by puromycin selection over a period of two-three weeks. Upon doxycycline induction, significant knock-down of NEK2 was observed in three NEK2 shRNA cell lines in both DF15 and DF15-PR background which results in significant decrease in cell proliferation in both DF15 and DF15-PR cell lines (data not shown). NEK2 shRNA cell lines in AMO1 and AMO1-PR background were also created and robust downregulation of NEK2 protein was observed upon induction in these two cell lines (data not shown). In both AMO1 and AMO1-PR cell lines, NEK2 knock-down resulted in a decrease in proliferation (data not shown). These results indicate that NEK2 knock-down results in reduced proliferation of both drug sensitive as well as drug-resistance cell lines.

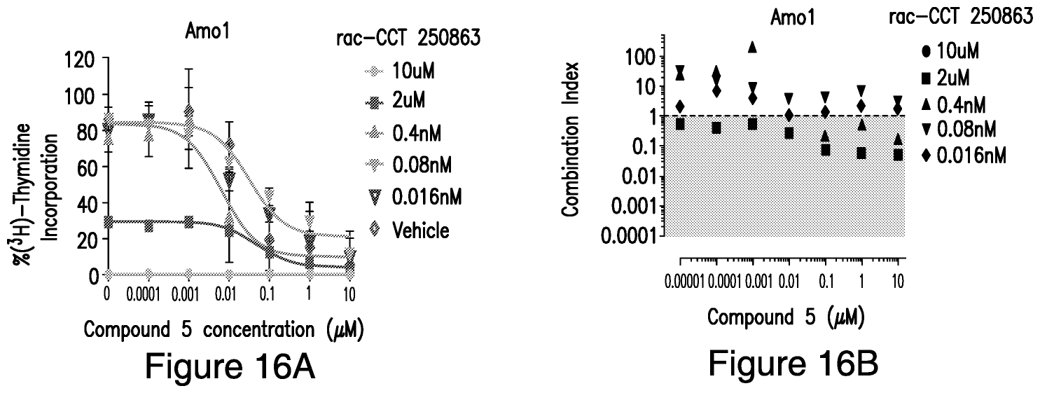

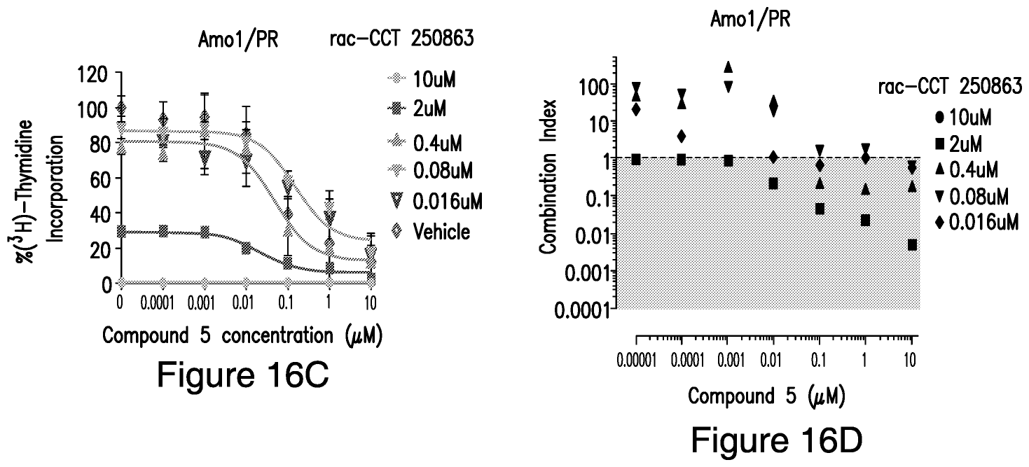

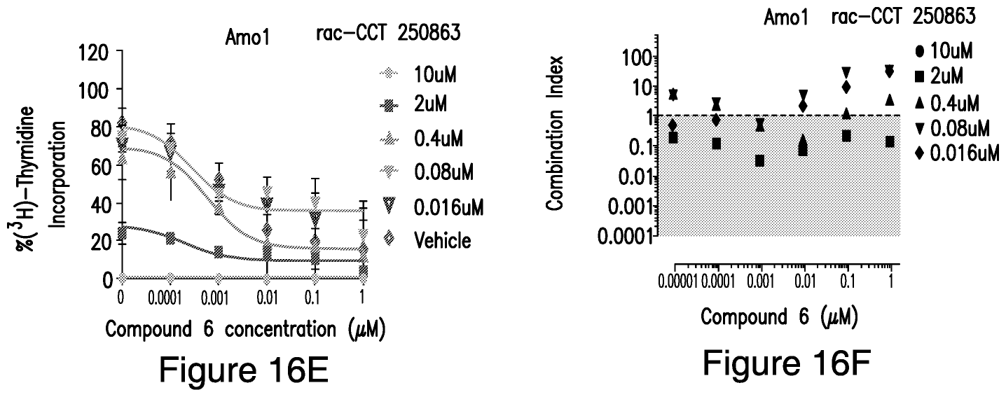

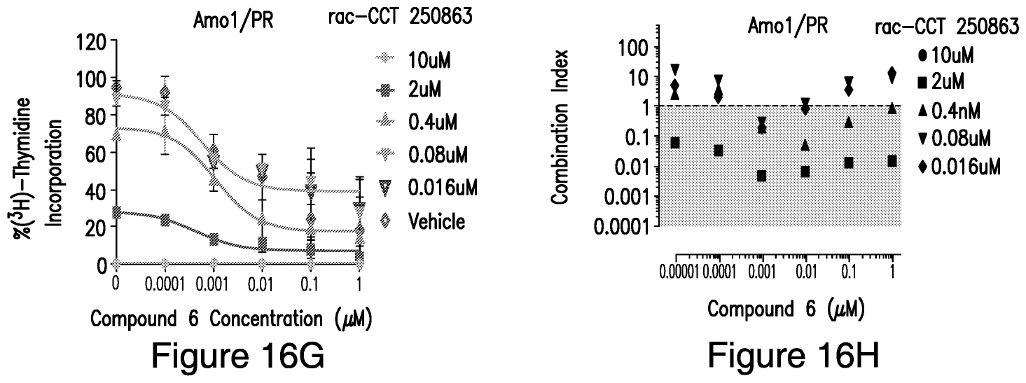

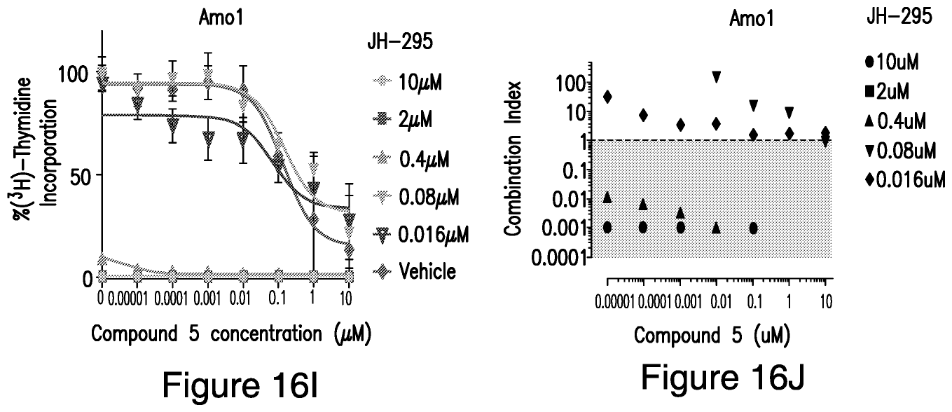

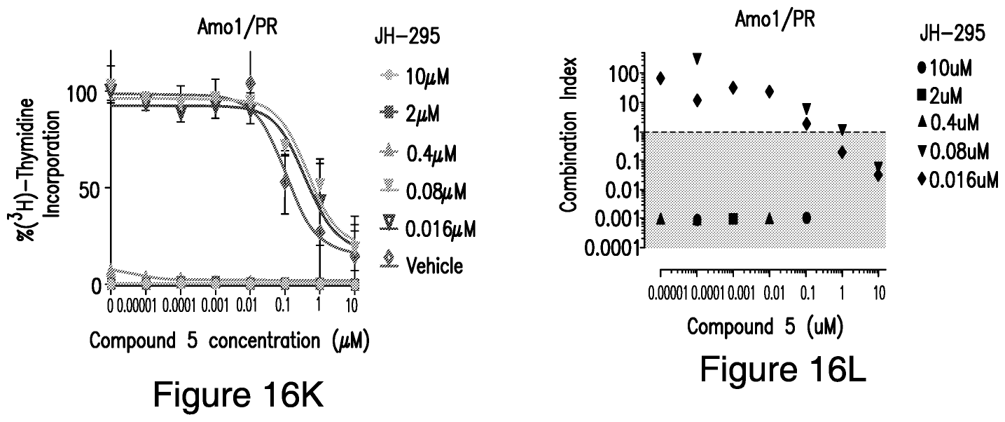

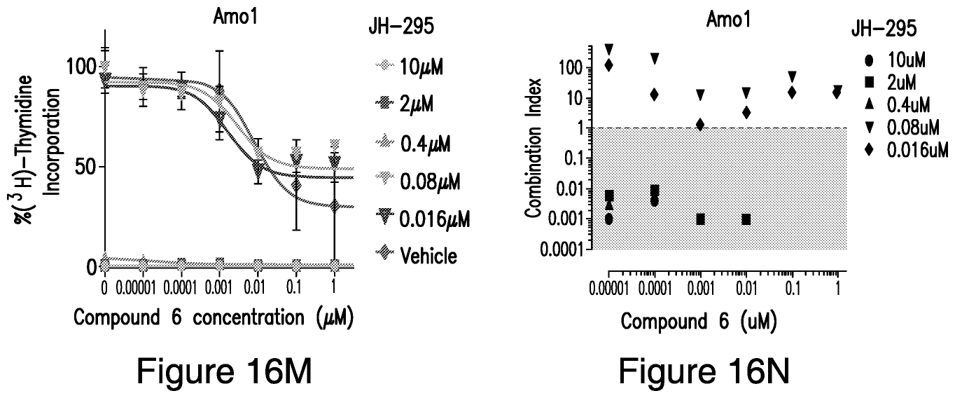

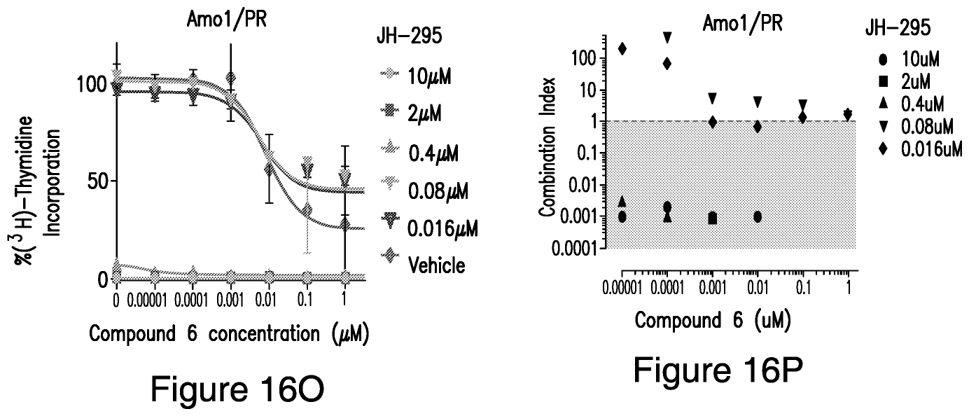

NEK2 inhibition exhibits strong synergy with antiproliferative compounds. Combination experiments using JH295 and rac-CCT 250863 inhibitors with Compound 5 and Compound 6 were performed. Five concentrations (0.016, 0.08, 0.4, 2 and 10 μM) of JH295 and Rac-CCT 250863 were combined with increasing concentrations of Compound 5 and Compound 6 and combination activity was studied in AMO1 and AMO1-PR cell lines. In both cell lines, the combinations of JH295 and Rac-CCT 250863 with Compound 5 and Compound 6 caused a concentration-dependent decrease in proliferation (FIGS. 16A, 16C, 16E, 16G, 16I, 16K, 16M, and 16O). The synergy of these combination datasets were analyzed using Calcusyn method and a strong synergy between NEK2 inhibitors (JH295 and rac-CCT 250863) with Compound 5 and Compound 6 (FIGS. 16B, 16D, 16F, 16H, 16J, 16L, 16N, and 16P) was found. It was further shown that NEK2 inhibitor and Compound 5 and Compound 6 combinations are more effective against drug resistant cell lines. Several more synergistic concentrations of NEK2i+ Compound 5 and Compound 6 combinations in AMO-PR cell lines were found in comparison to AMO1 lines. Similar experiments were repeated with MMS.1, K12PE and K12PE-PR cell lines and a strong synergy was observed between Compound 5 and Compound 6 and NEK2 inhibitors in MMS.1, K12PE and K12PE-PR cell lines (data not shown).

To further confirm the synergistic effect, shRNA knockdown was combined with either Compound 5 or Compound 6 treatment. Expression of control and NEK2 shRNA in AMO1 cell lines was induced, followed by exposure of the cells to increasing concentrations of Compound 5 and Compound 6. The results were measured through a proliferation assay. NEK2 knocked-down cells showed more vulnerability to Compound 5 and Compound 6 treatment. The combination of NEK2 knockdown increased the activity of Compound 5 by 5-fold (IC₅₀=0.1053 μM for Compound 5 in control cells vs. IC₅₀=0.01870 μM for Compound 5 in NEK2 knocked-down cells), and Compound 6 by 10 fold (IC₅₀=0.02965 μM for Compound 6 in control cells vs. IC₅₀=0.002892 μM for Compound 6 in NEK2 knocked-down cells) in comparison to control shRNA cell line.

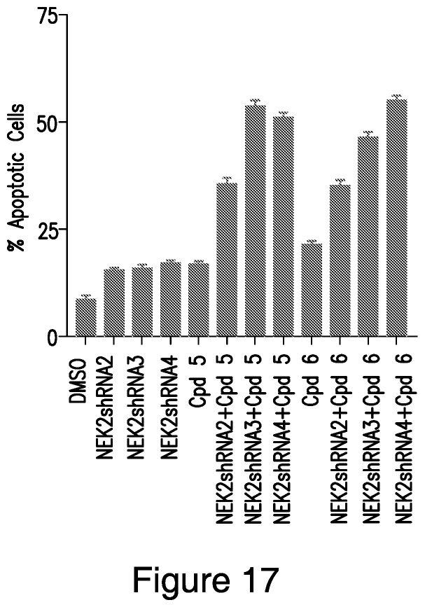

To further confirm the synergistic effect of NEK2 knockdown and Compound 5 and Compound 6 combination NEK2 knockdown cells were incubated with vehicle, Compound 5 and Compound 6, and the induction of apoptosis was measured by Annexin V staining. A strong increase in apoptotic cells was observed when NEK2 knock down was combined with Compound 5 or Compound 6 (FIG. 17). Quantification shows that NEK2 shRNA knockdown combined with Compound 5 or Compound 6 increases the percentage of apoptotic cell by 2-3 fold in comparison to DMSO control.

Effect of NEK2 downregulation on Compound 5 and Compound 6-induced substrate degradation. T Cells were treated with the combination of pomalidomide, Compound 5 and Compound 6 along with varying concentration of NEK2 inhibitor JH295 and substrate protein expression (Ikaros (IKZF1), Aiolos (IKZF3) and ZFP91) was analyzed by immunoblotting. No effect of the single agent NEK2 inhibitor JH295 on substrate degradation was observed in comparison to DMSO control. Similarly, a combination of pomalidomide, Compound 5 and Compound 6 with JH295 did not show any significant effect on substrate degradation. The effect of NEK2 knockdown on pomalidomide mediated substrate degradation was also studied. Control and NEK2 shRNA cells were incubated to varying concentrations of pomalidomide. Pomalidomide treatment degraded Ikaros (IKZF1), Aiolos (IKZF3) and ZFP91 in a concentration dependent manner in control shRNA lines. A similar pattern of substrate degradation was maintained in NEK2 knockdown cell lines. These experiments demonstrate that NEK2 knockdown do not affect the substrate degradation kinetics of Compound 5, Compound 6, and pomalidomide.

NEK2 knockdown and combination preferentially kills cells in G1/S phase of cell cycle. The cell cycle effect of NEK2 knockdown was analyzed. NEK2 activity is preferentially required in G2/M phase of cell cycle (Fry et al., J Cell Sci. 2012, 125(Pt 19):4423-4433) where it participates in centrosome separation (Hayward et al., Cancer Lett 237:155-166, 2006; O'regan et al., Cell Div. 2007, 2:25) and kinetochore microtubule attachment through HEC1 phosphorylation (RandyWei, Bryan Ngo, Guikai Wu, and Wen-Hwa Lee: Phosphorylation of the Ndc80 complex protein, HEC1, by Nek2 kinase modulates chromosome alignment and signaling of the spindle assembly checkpoint. (2011) Molecular Biology of the Cell 22:19, 3584-3594). The cell cycle profile of control and NEK2 shRNA cells were analyzed using PI staining. Simultaneously the percentage of apoptotic cells was measured by Annexin V staining of the same samples. An increase in apoptotic cells upon NEK2 shRNA induction in both drug sensitive and drug resistance cell lines was observed. No effect on cell cycle profile was observed (data not shown). NEK2 knockdown cells were cycling through cell cycle without any accumulation of cells in G2/M phase of cell cycle. The effect of NEK2 on mitosis was then explored using data from mitocheck (https://www.mitocheck.org/). Comparison of the data from PLK1 and NEK2 knockdown experiments in Hela cells showed that PLK1 knockdown gives strong prometaphase arrest and the cell undergoes apoptosis after a prolonged mitotic arrest and 100% cells follow a similar course of mitotic arrest and apoptosis (data not shown). The NEK2 knock down cell keeps cycling through cell cycle and intermittently undergoes apoptosis as evident by sudden induction nuclei fragmentation after few cell cycles. Three different phenotypes were observed in NEK2 knockdown cells: Phenotype 1: Generation of aneuploid cells. Phenotype 2: Following a normal cell cycle both the daughter cells undergo apoptosis in subsequent cell cycle. Phenotype 3: Following a normal cell cycle only a single daughter cells undergoes apoptosis in subsequent cell cycle.

Pomalidomide treatment combined with NEK2 inhibition increases apoptosis. Biparametric Annexin V and Propidium Iodide (PI) assay was performed to analyze cell cycle and apoptosis in the same sample and to quantify the proportion of cells undergoing apoptosis at each phase of cell cycle (Rieger et al., J Vis Exp. 2011, (50):2597; Léonce et al., Mol Pharmacol. 2001, 60(6):1383-1391). Control shRNA and NEK2 shRNA cell lines were treated with pomalidomide and followed cell cycle and apoptosis for the duration of two cell cycles. At 72 hours about 5.04% cells in the control shRNA lines were apoptotic compared to 21.1% apoptotic cells in NEK2 shRNA cell lines. This shows an enhanced pomalidomide induced apoptosis in NEK2 shRNA cell lines in comparison to control lines. Cell cycle and apoptosis analysis of the same samples shows that majority of apoptotic cells are contributed from G1-S phase of cell cycle. At 96 hours about 9.5% of the cells in control shRNA lines and 24.7% of the NEK2 shRNA cell lines were apoptotic, and a majority of apoptotic cells were again contributed from G1-S phase of cell cycle. This analysis shows that antiproliferative compounds such as pomalidomide mainly work at G1/S phase of cell cycle and NEK2 inhibition works G2/M phase of cell cycle. Combination of these two agents results in apoptosis of 20-25% cells in each cycle, with majority of apoptotic cells contributed from G1/S phase of cell cycle. As Taken together, the results show that NEK2 inhibitor treated and knock-down cells do not undergo mitotic arrest, but they undergo mitotic defect accumulation with time and finally undergoes apoptosis from G1/S phase of cell cycle due to pomalidomide treatment.

### Example 4

Methods and experimental information (e.g., proliferation assays, immunoblotting and flow to measure changes in proliferation, signaling and apoptosis) for this example are similar to those described in Example 1 for other targets.

Trametinib response correlates with p-ERK-1/2 level in MM cell lines irrespective of RAS/RAF mutation status. To analyze the relationship between p-ERK-1/2 expression and activity of trametinib, proliferation assays were performed in several MM cell lines with high p-ERK-1/2 expression (U266, H929, AMO1, MC-CAR, KARPAS-620, KMM-1, KMS-20, MOLP8) and low p-ERK-1/2 expression (K12PE, EJM, LP1, DF15, DF15PR, RPMI-8226). The results are shown in the tables below. Cell lines having higher p-ERK-1/2 expression were significantly more sensitive to Trametinib compared to those with low p-ERK-1/2 expression.

| | | | p-ERK | IC50 (μM) |
|---|---|---|---|---|
| SN | Cell Line | Mutation status | status | Trametinib |
| 1 | U266 | BRAF K601N | High | 0.000353 |
| 2 | H929 | NRAS G13D | Medium | 0.003276 |
| 3 | Amo1 | KRAS146T | High | 0.001203 |
| 4 | McCAR | WT | High | 0.003103 |
| 5 | KARPAS-620 | KRAS G12G | High | 0.000070 |
| 6 | KMM-1 | NRAS G13D | High | 0.00169 |
| 7 | KMS-20 | KRAS G12S | High | 0.002223 |
| 8 | MOLP-8 | KRAS K180del | High | 0.01169 |
| | | NRAS p.Q61L | | |

| | | | p-ERK | |
|---|---|---|---|---|
| SN | Cell Line | Mutation status | status | IC50 (μM) |
| 1 | K12PE | BRAF (Intronic) | low | — |
| 2 | EJM | WT | low | — |
| 3 | LP-1 | WT | low | — |
| 4 | DF15 | KRAS G12A | low | — |
| 5 | DF15PR | KRAS G12A | low | — |
| 6 | RPMI-8226 | KRAS G12A | low | — |

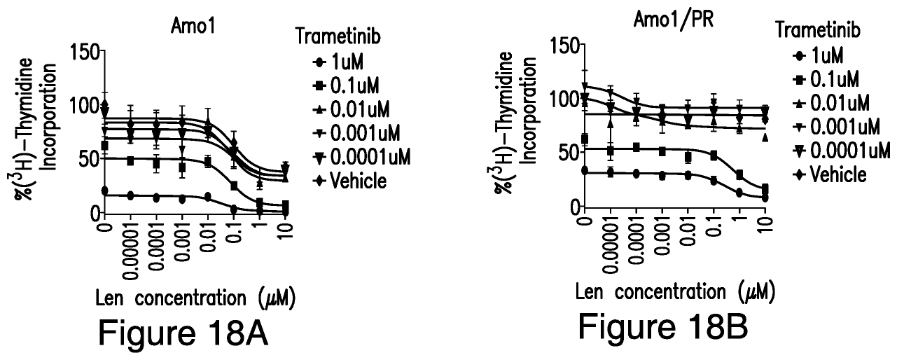

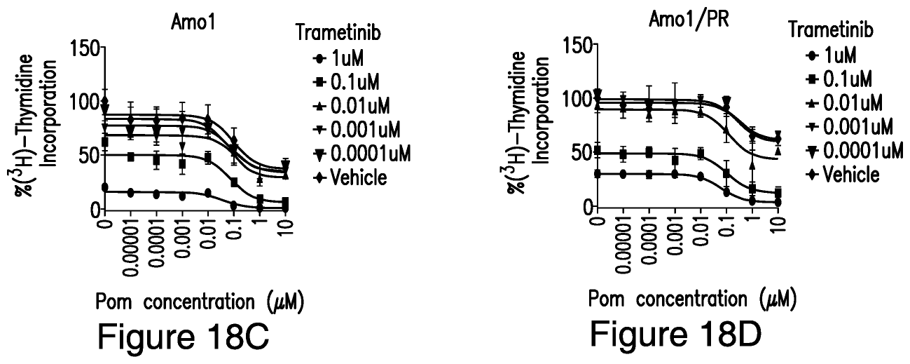

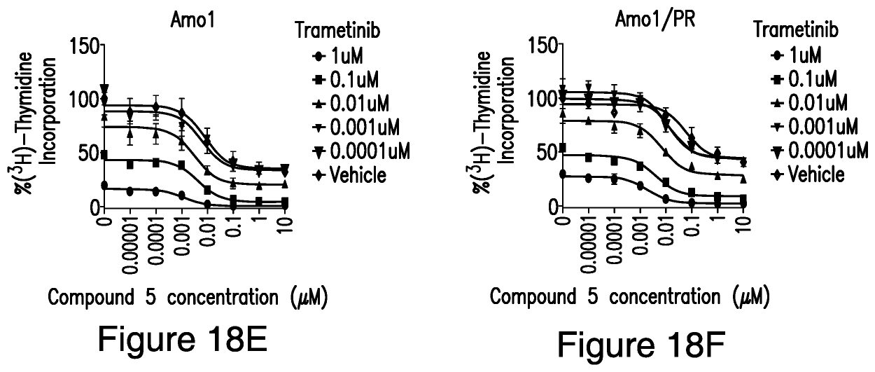

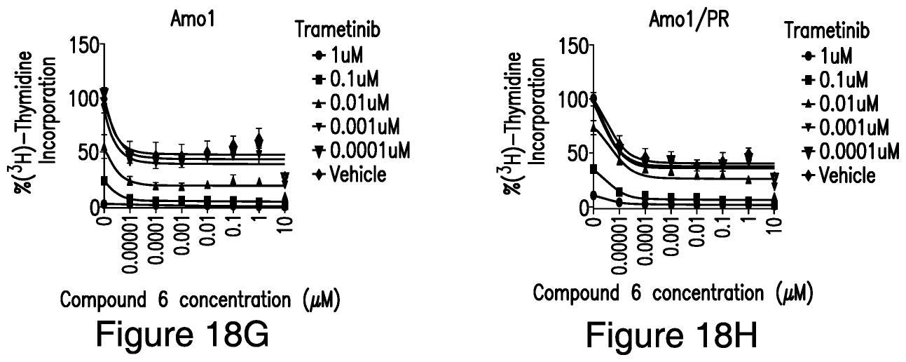

Trametinib shows synergy with immunomodulatory compounds, Compound 5 and Compound 6 in both pomalidomide sensitive and resistant cells. Proliferation assays were also performed to analyze the combinatorial activity of trametinib with immunomodulatory compounds (Len and Pom) or Compound 5 or Compound 6 in pomalidomide sensitive and pomalidomide resistant AMO1 and AMO1-PR cell lines. The results are shown in FIG. 18A to FIG. 18H. These proliferation assays demonstrated strong synergy of trametinib with immunomodulatory compounds, compound 5 and compound 6.

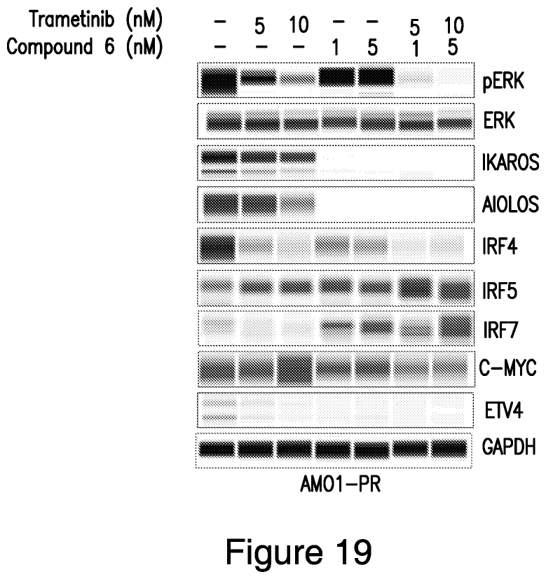

Trametinib and Compound 6 combination synergistically decreased ERK, ETV4 and MYC signaling in AMO1-PR cell line. In order to establish the mechanistic basis of the synergy between trametinib with Compound 6, immunoblotting was performed to detect changes in p-ERK, ETV4, AIOLOS, IKAROS, IRF4, IRF5, IRF7 and MYC signaling. The results are shown in FIG. 19. Combination of trametinib and Compound 6 demonstrated a greater decrease in p-ERK, ETV4, MYC and IRF4 levels and an increase in the levels of Interferon genes IRF5 and IRF7 compared to the monotherapies.

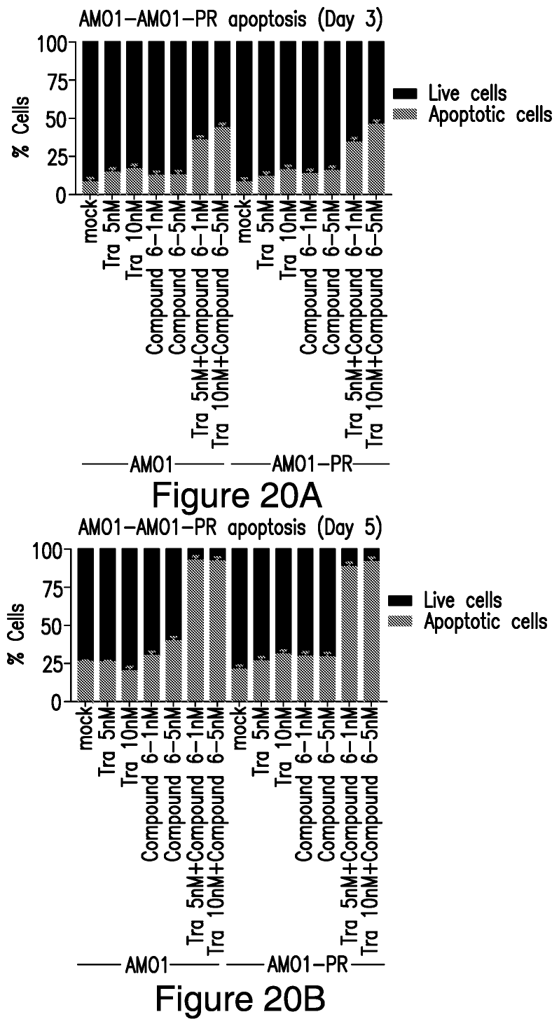

Trametinib and Compound 6 combination increased apoptosis in AMO1 and AMO1-PR cell line. The effects of trametinib and Compound 6 combination on apoptosis were further analyzed at Day 3 and Day 5 in AMO1 and AMO1-PR cell lines. In both of these cell lines, combination of trametinib and Compound 6 showed higher apoptosis at Day 3 (FIG. 20A) and Day 5 (FIG. 20B) compared to the monotherapies.

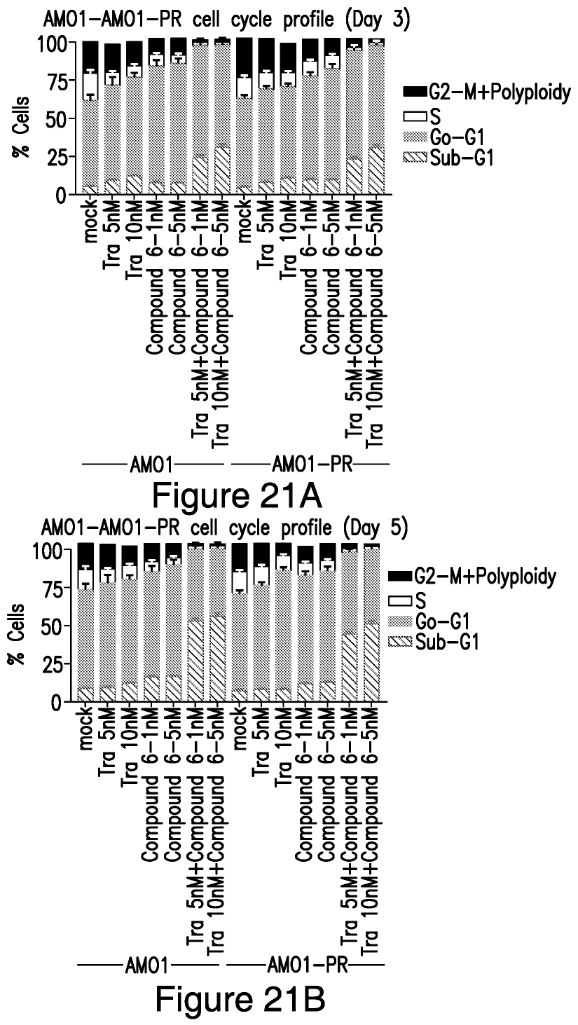

Trametinib and Compound 6 combination decreased G2-M and S phase cells in AMO1 and AMO1-PR cell lines. To analyze the cell cycle associated mechanisms of synergy between trametinib and Compound 6, cell cycle studies were performed in response to the combination and monotherapies. Cell cycle results demonstrated a greater decrease in G2-M and S phases of cell cycle in response to the combination compared to the monotherapies at Day 3 (FIG. 21A) and Day 5 (FIG. 21B).

### Example 5

Methods and experimental information (e.g., proliferation assays, immunoblotting and flow to measure changes in proliferation, signaling and apoptosis) for this example are similar to those described in Example 1 for other targets.

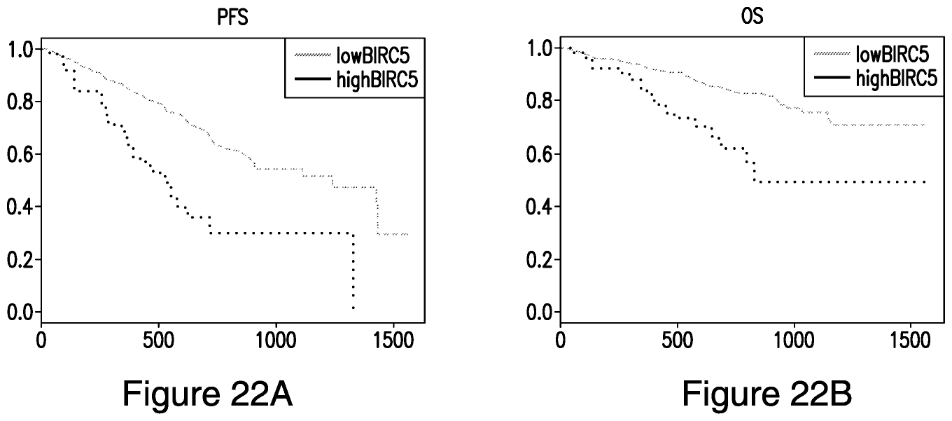

BIRC5 inhibitor, YM155 decreases proliferation of both Pom sensitive and resistant cell lines. MM patients in myeloma genome project (the data were derived from the Myeloma XI trial, the Dana-Faber Cancer Institute/Intergroupe Francophone du Myelome, and the Multiple Myeloma Research Foundation CoMMpass study, which have been reported) with high expression of BIRC5 demonstrated poorer PFS (FIG. 22A) and OS (FIG. 22B). Treatments of AMO1, AMO1-PR, K12PE, K12PE-PR cell lines with BIRC5 inhibitor YM155 demonstrated higher sensitivity of AMO1-PR (EC₅₀=0.12 nM) and K12PE-PR (EC₅₀=1.07 nM) to BIRC5 inhibitor compared to the parental cell lines AMO1 (EC₅₀=1.09 nM) and K12PE (EC₅₀=1.47 nM).

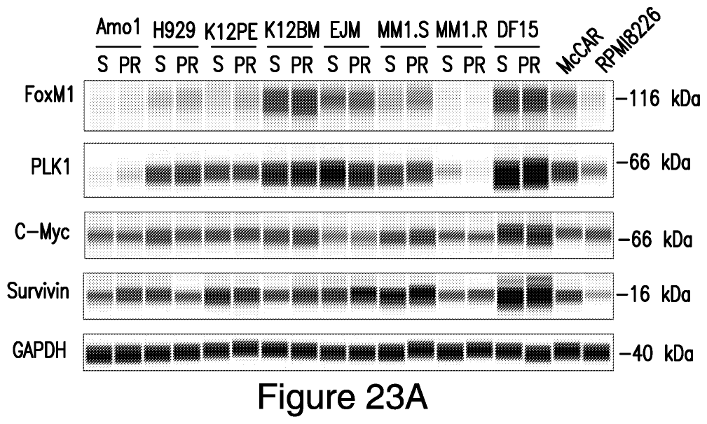

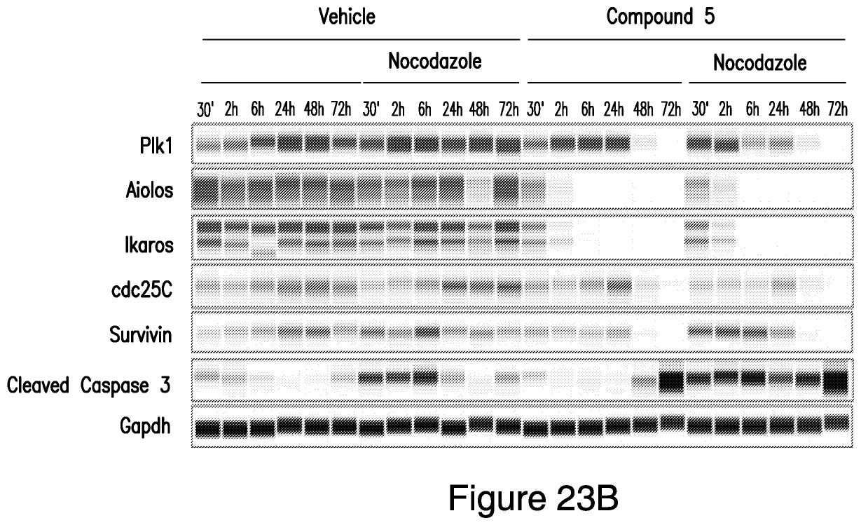

BIRC5 (Survivin) is downregulated in response to Compound 5 leading to late apoptosis. BIRC5 expression was studied in MM isogenic pomalidomide sensitive and resistant cell lines and several pomalidomide resistant cell lines demonstrated increase expression of BIRC5 (FIG. 23A). BIRC5 levels decreased in response to Compound 5 treatment at 48 and 72 hours, followed by an onset of apoptosis in MM1.S cell line (FIG. 23B).

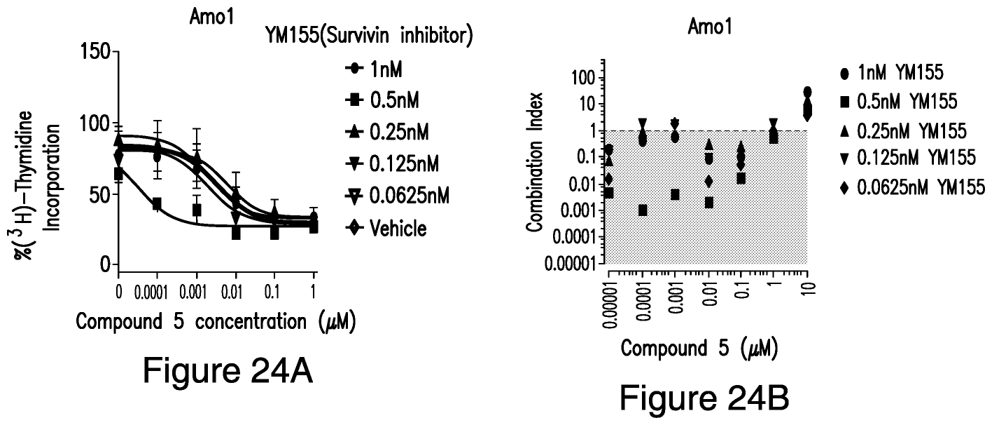

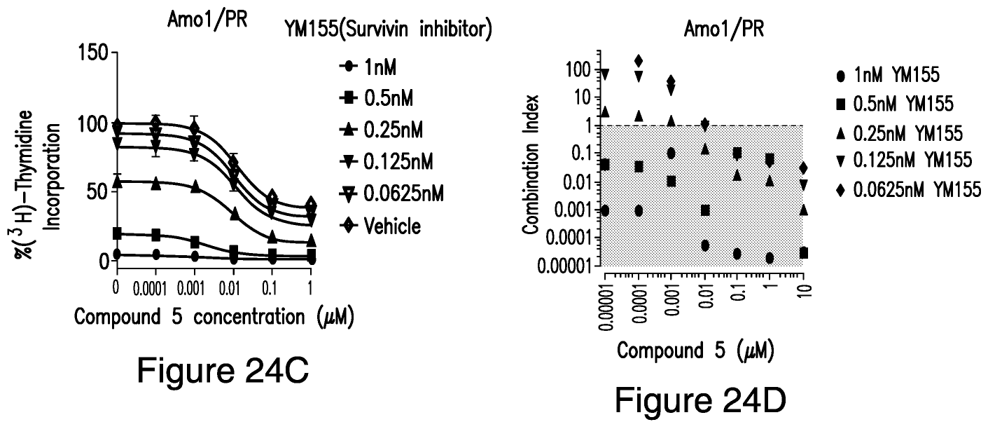

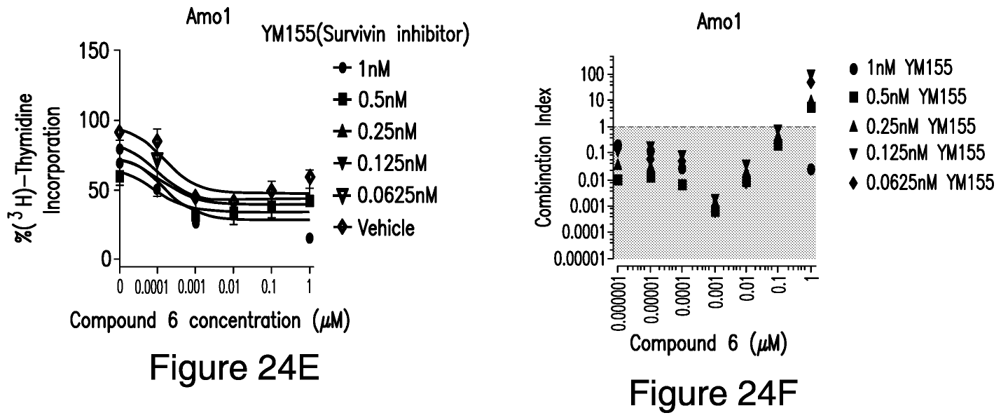

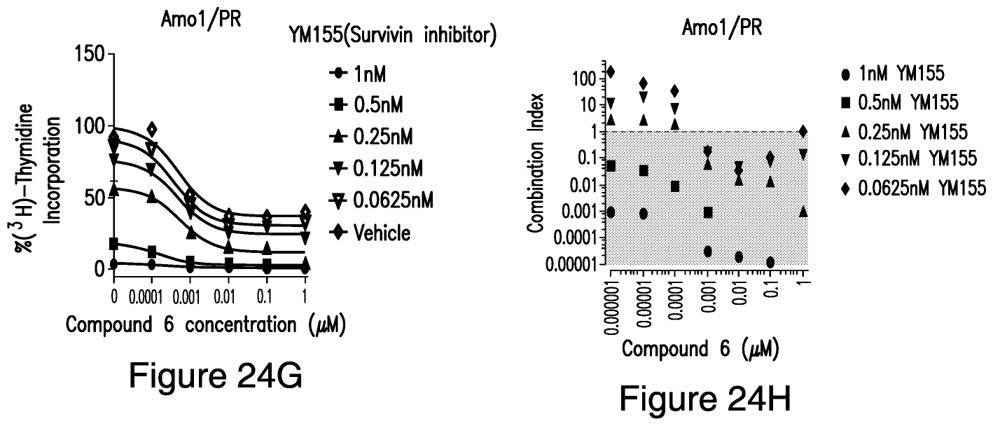

YM155 and Compound 5 or Compound 6 synergistically decrease proliferation in pomalidomide sensitive and resistant cell lines. AMO1 and AMO1-PR cell lines were treated with increasing doses of YM155 and Compound 5 or Compound 6 and proliferation assays were performed. The results are shown in FIG. 24A to FIG. 24H. Combination analysis using Calcusyn showed the synergistic activity of YM155 with Compound 5 or Compound 6 in both AMO1 and AMO1-PR cell lines.

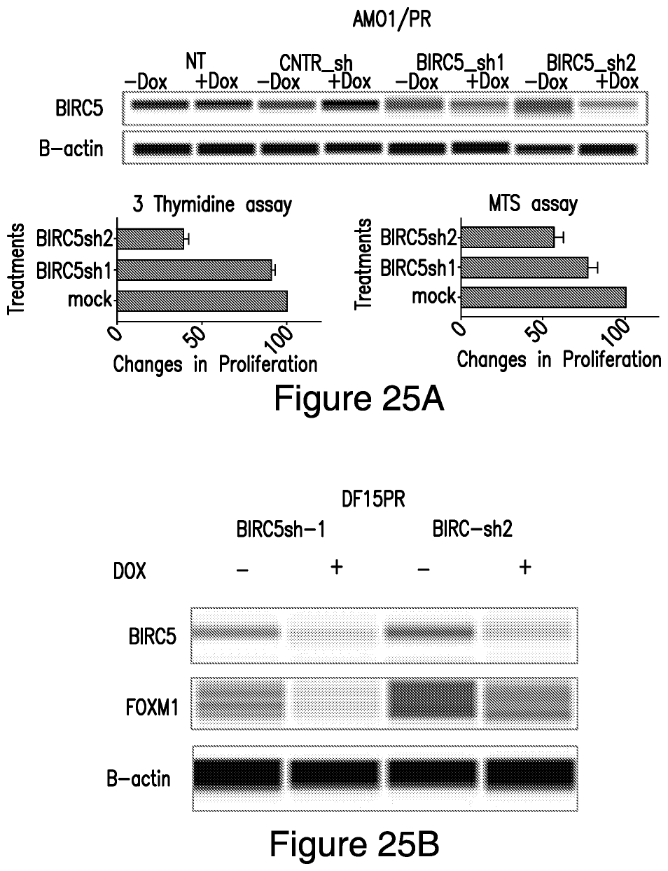

Knockdown of BIRC5 Decreases Proliferation in MM Cell Lines. Doxycycline inducible BIRC5 knock-down cell lines were developed. BIRC5 knock-down decreased the proliferation of AMO1-PR cells (FIG. 25A). BIRC5 knock-down also downregulated the expression of high risk associated gene, FOXM1 (FIG. 25B).

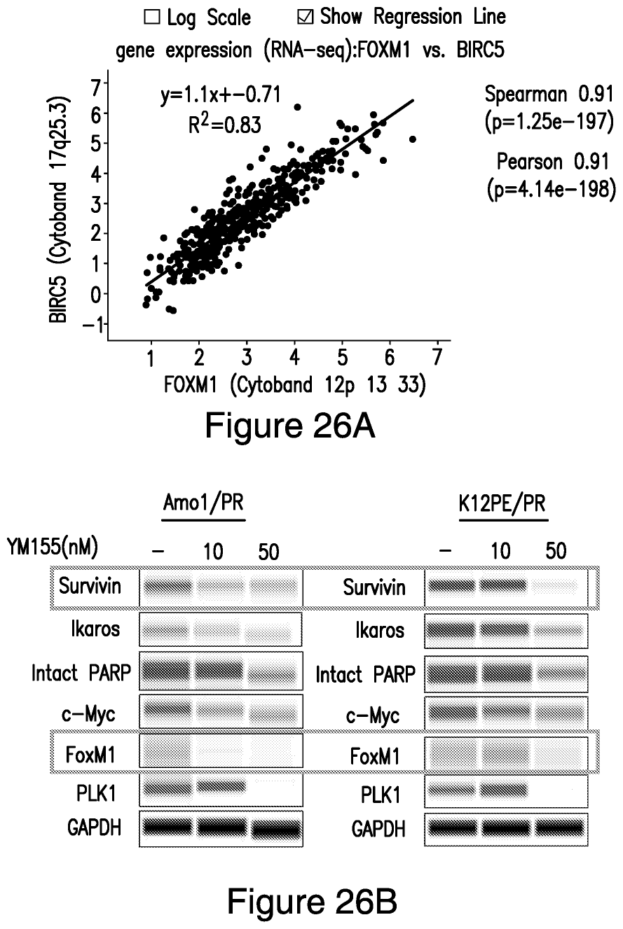

Inhibition of BIRC5 by YM155 also downregulates FOXM1 and pro-survival signaling. High risk associated genes, BIRC5 and FOXM1 demonstrated significant co-expression in myeloma genome project, suggesting their co-regulation (FIG. 26A). Inhibition of BIRC5 by YM155 downregulated FOXM1 expression in a dose dependent manner in AMO1-PR and K12PE-PR cell lines (FIG. 26B).

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

A number of references have been cited, the disclosures of which are incorporated herein by reference in their entirety.

What is claimed is:

1. A method of treating cancer, comprising administering to a patient in need thereof a therapeutically effective amount of (S)-3-(4-((4-(morpholinomethyl)benzyl)oxy)-1-oxoisoindolin-2-yl)piperidine-2,6-dione (Compound 5), or a stereoisomer or mixture of stereoisomers, pharmaceutically acceptable salt, tautomer, prodrug, solvate, hydrate, co-crystal, clathrate, or polymorph thereof in combination with a second agent, wherein the second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRC5 inhibitor, or a DNA methyltransferase inhibitor.

2. A method of treating cancer, comprising administering to a patient in need thereof a therapeutically effective amount of (S)-4-(4-(4-(((2-(2,6-dioxopiperidin-3-yl)-1-oxoisoindolin-4-yl)oxy)methyl)benzyl)piperazin-1-yl)-3-fluorobenzonitrile (Compound 6), or a stereoisomer or mixture of stereoisomers, pharmaceutically acceptable salt, tautomer, prodrug, solvate, hydrate, co-crystal, clathrate, or polymorph thereof in combination with a second agent, wherein the second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRC5 inhibitor, or a DNA methyltransferase inhibitor.

3. A method of treating cancer, comprising administering to a patient in need thereof a therapeutically effective amount of 4-amino-2-(2,6-dioxopiperidine-3-yl)isoindoline-1,3-dione (Compound 1), or a stereoisomer or mixture of stereoisomers, pharmaceutically acceptable salt, tautomer, prodrug, solvate, hydrate, co-crystal, clathrate, or polymorph thereof in combination with a second agent, wherein the second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRC5 inhibitor, or a DNA methyltransferase inhibitor.

4. A method of treating cancer, comprising administering to a patient in need thereof a therapeutically effective amount of 3-(4-amino-1-oxo-1,3 dihydro-isoindol-2-yl)-piperidine-2,6-dione (Compound 2), or a stereoisomer or mixture of stereoisomers, pharmaceutically acceptable salt, tautomer, prodrug, solvate, hydrate, co-crystal, clathrate, or polymorph thereof in combination with a second agent, wherein the second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRC5 inhibitor, or a DNA methyltransferase inhibitor.

5. A method of treating cancer, comprising administering to a patient in need thereof a therapeutically effective amount of 2-(2,6-dioxo-3-piperidinyl)-1H-isoindole-1,3(2H)-dione (Compound 3), or a stereoisomer or mixture of stereoisomers, pharmaceutically acceptable salt, tautomer, prodrug, solvate, hydrate, co-crystal, clathrate, or polymorph thereof in combination with a second agent, wherein the second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRC5 inhibitor, or a DNA methyltransferase inhibitor.

6. A method of treating cancer, comprising administering to a patient in need thereof a therapeutically effective amount of 3-(5-amino-2-methyl-4-oxo-4H-quinazolin-3-yl)-piperidine-2,6-dione (Compound 4), or a stereoisomer or mixture of stereoisomers, pharmaceutically acceptable salt, tautomer, prodrug, solvate, hydrate, co-crystal, clathrate, or polymorph thereof in combination with a second agent, wherein the second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRC5 inhibitor, or a DNA methyltransferase inhibitor.

7. A method of treating cancer, comprising administering to a patient in need thereof a therapeutically effective amount of 2-(4-chlorophenyl)-N-((2-(2,6-dioxopiperidin-3-yl)-1-oxoisoindolin-5-yl)methyl)-2,2-difluoroacetamide (Compound 7), or a stereoisomer or mixture of stereoisomers, pharmaceutically acceptable salt, tautomer, prodrug, solvate, hydrate, co-crystal, clathrate, or polymorph thereof in combination with a second agent, wherein the second active agent is one or more of a PLK1 inhibitor, a BRD4 inhibitor, a BET inhibitor, an NEK2 inhibitor, an AURKB inhibitor, an MEK inhibitor, a PHF19 inhibitor, a BTK inhibitor, an mTOR inhibitor, a PIM inhibitor, an IGF-1R inhibitor, an XPO1 inhibitor, a DOT1L inhibitor, an EZH2 inhibitor, a JAK2 inhibitor, a BIRC5 inhibitor, or a DNA methyltransferase inhibitor.

8. The method of any one of claims 1 to 7, wherein the second agent is a PLK1 inhibitor.

9. The method of claim 8, wherein the PLK1 inhibitor is BI2536, volasertib, CYC140, onvansertib, GSK461364, or TAK960, or a stereoisomer, mixture of stereoisomers, tautomer, isotopolog, or pharmaceutically acceptable salt thereof.

10. The method of claim 9, wherein the PLK1 inhibitor is BI2536.

11. The method of any one of claims 1 to 7, wherein the second agent is a BRD4 inhibitor.

12. The method of claim 11, wherein the BRD4 inhibitor is JQ1.

13. The method of any one of claims 1 to 7, wherein the second agent is a BET inhibitor.

14. The method of claim 13, wherein the BET inhibitor is birabresib, 4-[2-(cyclopropylmethoxy)-5-(methanesulfonyl)phenyl]-2-methylisoquinolin-1(2H)-one (Compound A), BMS-986158, RO-6870810, CPI-0610, or molibresib, or a stereoisomer, mixture of stereoisomers, tautomer, isotopolog, or pharmaceutically acceptable salt thereof.

15. The method of any one of claims 1 to 7, wherein the second agent is an NEK2 inhibitor.

16. The method of claim 15, wherein the NEK2 inhibitor is JH-295.

17. The method of claim 15, wherein the NEK2 inhibitor is rac-CCT 250863.

18. The method of any one of claims 1 to 7, wherein the second agent is an Aurora Kinase B (AURKB) inhibitor.

19. The method of claim 18, wherein the AURKB inhibitor is barasertib, AZD1152-HQPA, alisertib, danusertib, AT9283, PF-03814735, AMG900, tozasertib, ZM447439, MLN8054, hesperidin, SNS-314, PHA-680632, CYC116, GSK1070916, TAK-901, or CCT137690, or molibresib, or a stereoisomer, mixture of stereoisomers, tautomer, isotopolog, or pharmaceutically acceptable salt thereof.

20. The method of any one of claims 1 to 7, wherein the second agent is a MEK inhibitor.

21. The method of claim 20, wherein the MEK inhibitor interrupts the function of the RAF/RAS/MEK signal transduction cascade.

22. The method of claim 20, wherein the MEK inhibitor is trametinib, trametinib dimethyl sulfoxide, cobimetinib, binimetinib, or selumetinib, or a stereoisomer, mixture of stereoisomers, tautomer, isotopolog, or pharmaceutically acceptable salt thereof.

23. The method of any one of claims 1 to 7, wherein the second agent is a PHF19 inhibitor.

24. The method of any one of claims 1 to 7, wherein the second active agent is a BTK inhibitor.

25. The method of claim 24, wherein the BTK inhibitor is ibrutinib, or acalabrutinib, or a stereoisomer, mixture of stereoisomers, tautomer, isotopolog, or pharmaceutically acceptable salt thereof.

26. The method of any one of claims 1 to 7, wherein the second active agent is an mTOR inhibitor.

27. The method of claim 26, wherein the mTOR inhibitor is rapamycin or an analog thereof (also termed rapalog).

28. The method of claim 26, wherein the mTOR inhibitor is everolimus.

29. The method of any one of claims 1 to 7, wherein the second active agent is a PIM inhibitor.

30. The method of claim 29, wherein the PIM inhibitor is LGH-447, AZD1208, SGI-1776, or TP-3654, or a stereoisomer, mixture of stereoisomers, tautomer, isotopolog, or pharmaceutically acceptable salt thereof.

31. The method of any one of claims 1 to 7, wherein the second active agent is an IGF-1R inhibitor.

32. The method of claim 31, wherein the IGF-1R inhibitor is linsitinib.

33. The method of any one of claims 1 to 7, wherein the second active agent is an XPO1 inhibitor.

34. The method of claim 33, wherein the XPO1 inhibitor is selinexor.

35. The method of any one of claims 1 to 7, wherein the second active agent is a DOT1L inhibitor.

36. The method of claim 35, wherein the DOT1L inhibitor is SGC0946, or pinometostat, or a stereoisomer, mixture of stereoisomers, tautomer, isotopolog, or pharmaceutically acceptable salt thereof.

37. The method of any one of claims 1 to 7, wherein the second active agent is an EZH2 inhibitor.

38. The method of claim 37, wherein the EZH2 inhibitor is tazemetostat, 3-deazaneplanocin A (DZNep), EPZ005687, EI1, GSK126, UNC1999, CPI-1205, or sinefungin, or a stereoisomer, mixture of stereoisomers, tautomer, isotopolog, or pharmaceutically acceptable salt thereof.

39. The method of any one of claims 1 to 7, wherein the second active agent is a JAK2 inhibitor.

40. The method of claim 39, wherein the JAK2 inhibitor is fedratinib, ruxolitinib, baricitinib, gandotinib, lestaurtinib, momelotinib, or pacritinib, or a stereoisomer, mixture of stereoisomers, tautomer, isotopolog, or pharmaceutically acceptable salt thereof.

41. The method of any one of claims 1 to 7, wherein the second active agent is a BIRC5 inhibitor.

42. The method of claim 41, wherein the BIRC5 inhibitor is YM155.

43. The method of any one of claims 1 to 7, wherein the second active agent is a DNA methyltransferase inhibitor.

44. The method of claim 43, wherein the DNA methyltransferase inhibitor is azacitidine.

45. The method of any one of claims 1 to 44, wherein the cancer is a hematological malignancy.

46. The method of any one of claims 1 to 44, wherein the cancer is a B-cell malignancy.

47. The method of any one of claims 1 to 44, wherein the cancer is lymphoma.

48. The method of any one of claims 1 to 44, wherein the cancer is diffuse large B-cell lymphoma (DLBCL).

49. The method of any one of claims 1 to 44, wherein the cancer is Mantle Cell Lymphoma (MCL).

50. The method of any one of claims 1 to 44, wherein the cancer is Marginal Zone Lymphoma (MZL).

51. The method of any one of claims 1 to 44, wherein the cancer is indolent follicular cell lymphoma (iFCL).

52. The method of any one of claims 1 to 44, wherein the cancer is T-cell lymphoma.

53. The method of any one of claims 1 to 44, wherein the cancer is multiple myeloma.

54. The method of claim 53, wherein the multiple myeloma is relapsed or refractory.

55. The method of claim 53, wherein the multiple myeloma is refractory to lenalidomide.

56. The method of claim 53, wherein the multiple myeloma is newly diagnosed multiple myeloma.

57. The method of claim 53, wherein the multiple myeloma is refractory to pomalidomide.

58. The method of claim 57, wherein the multiple myeloma is refractory to pomalidomide when used in combination with a proteasome inhibitor.

59. The method of claim 58, wherein the proteasome inhibitor is selected from bortezomib, carfilzomib, and ixazomib.

60. The method of claim 57, wherein the multiple myeloma is refractory to pomalidomide when used in combination with an inflammatory steroid.

61. The method of claim 60, wherein the inflammatory steroid is selected from dexamethasone or prednisone.

62. The method of claim 57, wherein the multiple myeloma is refractory to pomalidomide when used in combination with a CD38 directed monoclonal antibody.

63. The method of any one of claims 1 to 62, additionally comprising administering to the patient an additional active agent.

64. The method of claim 63, wherein the third agent is a steroid.

65. A method of identifying a subject having a hematological cancer who is likely to be responsive to a treatment compound in combination with a second agent, or predicting the responsiveness of a subject having a hematological cancer to a treatment compound in combination with a second agent, comprising:
a. obtaining a sample from the subject;
b. determining a biomarker level in the sample; and
c. diagnosing the subject as being likely to be responsive to the treatment compound in combination with the second agent if the biomarker level is an altered level relative to a reference level of the biomarker.

66. A method of selectively treating a hematological cancer in a subject having a hematological cancer, comprising:
a. obtaining a sample from the subject;
b. determining a biomarker level in the sample;
c. diagnosing the subject as being likely to be responsive to the treatment compound in combination with the second agent if the biomarker level is an altered level relative to a reference level of the biomarker; and
d. administering a therapeutically effective amount of the treatment compound in combination with the second agent to the subject diagnosed as being likely to be responsive to the treatment compound in combination with the second agent.

67. The method of claim 65 or 66, wherein the biomarker is expression of a gene or a combination of genes selected from: BRD4, PLK1, AURKB, PHF19, NEK2, MEK, BTK, MTOR, PIM, IGF-1R, XPO1, DOT1L, EZH2, JAK2, and BIRC5.
